SEQUENCE LISTING (280 sequences)

SEQ ID NO: 1 - protein, 120 aa, synthetic construct
QVQLVESGAEVKKPGASVRLSCKASGYSFIHYYLHYVRQAPGQGLEWMGVINPFDGSTDYAQKFHGRVTLTRDTSASTVFMELSSLGSEDTAVYFCTRDNMIGYVLFDYWGLGTLVTVSS

SEQ ID NO: 2 - protein, 5 aa, synthetic construct
HYYLH

SEQ ID NO: 3 - protein, 17 aa, synthetic construct
VINPFDGSTDYAQKFHG

SEQ ID NO: 4 - protein, 11 aa, synthetic construct
DNMIGYVLFDY

SEQ ID NO: 5 - protein, 120 aa, synthetic construct
QVQLVQSGAEVKKPGASVKVSCKTSGYTFTRYYIQWVRQAPGQGLEWMGVINPYDGSTNYAQKFQGRVTVTRDTSTFTVYMELSSLRSEDTAVYYCARDYMRGYVIFDYWGQGTLVTVSS

SEQ ID NO: 6 - protein, 5 aa, synthetic construct
RYYIQ

SEQ ID NO: 7 - protein, 17 aa, synthetic construct
VINPYDGSTNYAQKFQG

SEQ ID NO: 8 - protein, 11 aa, synthetic construct
DYMRGYVIFDY

SEQ ID NO: 9 - protein, 120 aa, synthetic construct
QVQLVQSGAEVKKPGASVKVSCKASGYTFNRHYIQWVRQAPGQGLEWMGTINPYDGSTHYAQKFQGRVTLTRDTSTTTVYMELSSLRSEDTAVYYCARDYMIGYVVFDYWGQGTLVTVSS

SEQ ID NO: 10 - protein, 5 aa, synthetic construct
RHYIQ

SEQ ID NO: 11 - protein, 17 aa, synthetic construct
TINPYDGSTHYAQKFQG

SEQ ID NO: 12 - protein, 11 aa, synthetic construct
DYMIGYVVFDY

SEQ ID NO: 13 - protein, 120 aa, synthetic construct
QVQLVQSGAEVKKPGASLKVSCKASGYTFTNYYIQWVRQAPGQGLEWMGVINPYDGSTNYAQKFQGRVTLTRDTSTTTVYMELSSLRSDDTAVYYCARDYMIGYVVFDYWGQGTLVTVSS

SEQ ID NO: 14 - protein, 5 aa, synthetic construct
NYYIQ

SEQ ID NO: 15 - protein, 17 aa, synthetic construct
VINPYDGSTNYAQKFQG

SEQ ID NO: 16 - protein, 11 aa, synthetic construct
DYMIGYVVFDY

SEQ ID NO: 17 - protein, 120 aa, synthetic construct
QVQLVQSGAEVKKPGASVRVSCKASGYPFINYYIQWVRQAPGQGLEWMGVVNPYDGSTNYAQKFQGRVSITRDASTSTVFMELNSLRSEDTAVYYCARDSMLGHVIFDFWGQGTLVTVSS

SEQ ID NO: 18 - protein, 5 aa, synthetic construct
NYYIQ

SEQ ID NO: 19 - protein, 17 aa, synthetic construct
VVNPYDGSTNYAQKFQG

SEQ ID NO: 20 - protein, 11 aa, synthetic construct
DSMLGHVIFDF

SEQ ID NO: 21 - protein, 120 aa, synthetic construct
QVQLVQSGAEVRKPGASVKFSCKASGYSFTHYYVHWVRQAPGQGLEWMGVINPFDSSTNYARKFQGRVTMTRDTSTTTVYMDLSSLRSEDTAVYFCARDAMEGDSYFDYWGQGTLVTVSS

SEQ ID NO: 22 - protein, 5 aa, synthetic construct
HYYVH

SEQ ID NO: 23 - protein, 17 aa, synthetic construct
VINPFDSSTNYARKFQG

SEQ ID NO: 24 - protein, 11 aa, synthetic construct
DAMEGDSYFDY

SEQ ID NO: 25 - protein, 120 aa, synthetic construct
QVQLVESGAEVKKPGASVKVSCKASGYTFARYYVHWVRQAPGQGLEWMGLINPYGGATNYAQNFQGRVTMTRDTSTSTVYMELSSLRSEDTAVYYCARDAMGGDSYFDYWGQGTLVTVSS

SEQ ID NO: 26 - protein, 5 aa, synthetic construct
RYYVH

SEQ ID NO: 27 - protein, 17 aa, synthetic construct
LINPYGGATNYAQNFQG

SEQ ID NO: 28 - protein, 11 aa, synthetic construct
DAMGGDSYFDY

SEQ ID NO: 29 - protein, 120 aa, synthetic construct
QVQLVQSGAEVKKPGASVKVSCKASGYSFIHYYIHWVRQAPGQGLEWMGVINPFDGSTNYAQKFQDRVTVTRDTSTSTVFMDLSSLRSEDTAVYYCARDNMIGYVLFDDWGQGTLVTVSS

SEQ ID NO: 30 - protein, 5 aa, synthetic construct
HYYIH

SEQ ID NO: 31 - protein, 17 aa, synthetic construct
VINPFDGSTNYAQKFQD

SEQ ID NO: 32 - protein, 11 aa, synthetic construct
DNMIGYVLFDD

SEQ ID NO: 33 - protein, 120 aa, synthetic construct
QVQLVQSGAEVKKPGASVRVSCKASGYPFINYYIQWVRQAPGQGLEWMGVINPFDGSTNYAQKFQDRVTVTRDTSTSTVFMDLSSLRSEDTAVYYCARDNMIGYVLLDYWGQGTLVTVSS

SEQ ID NO: 34 - protein, 5 aa, synthetic construct
NYYIQ

SEQ ID NO: 35 - protein, 17 aa, synthetic construct
VINPFDGSTNYAQKFQD

SEQ ID NO: 36 - protein, 11 aa, synthetic construct
DNMIGYVLLDY

SEQ ID NO: 37 - protein, 120 aa, synthetic construct
QVQLVQSGAEVKKPGASVTVSCKTSGYTFTSYYIHWLRQAPGQGLEWMGVINPFGGATNYAQKFQGRVTMTRDTSTITVYMELSSLRSEDTAVYYCARDAMGGDSHFDHWGRGTLVTVSS

SEQ ID NO: 38 - protein, 5 aa, synthetic construct
SYYIH

SEQ ID NO: 39 - protein, 17 aa, synthetic construct
VINPFGGATNYAQKFQG

SEQ ID NO: 40 - protein, 11 aa, synthetic construct
DAMGGDSHFDH

SEQ ID NO: 41 - protein, 120 aa, synthetic construct
QVQLVQSGAEVKEPGASMTLSCKTSGYTFISNYIHWLRQAPGQGLEWLGVINPFGGATNYAQKFQGRVTMTRDTSTITVYMELSSLRSEDTAVYYCARDAMGGDSHFDYWGQGTLVTVSS

SEQ ID NO: 42 - protein, 5 aa, synthetic construct
SNYIH

SEQ ID NO: 43 - protein, 17 aa, synthetic construct
VINPFGGATNYAQKFQG

SEQ ID NO: 44 - protein, 11 aa, synthetic construct
DAMGGDSHFDY

SEQ ID NO: 45 - protein, 120 aa, synthetic construct
QVQLVESGTEVKKPGASVKVSCKASGYSFIHYYIHWVRQAPGRGLEWMGVINPFDGSTNSAQNFQGRVTVTRDTSTSTVFMDLSSLRSEDTAVYYCARDNMLGYVLFDHWGQGTLVTVSS

SEQ ID NO: 46 - protein, 5 aa, synthetic construct
HYYIH

SEQ ID NO: 47 - protein, 17 aa, synthetic construct
VINPFDGSTNSAQNFQG

SEQ ID NO: 48 - protein, 11 aa, synthetic construct
DNMLGYVLFDH

SEQ ID NO: 49 - protein, 120 aa, synthetic construct
QVQLVESGAEVKKPGASVKVSCKASGYTFTRYYLHWVRQAPGQGLEWMGVINPYGGATNYAQKFQGRVTLTRDTSTSTVYMELNSLRSEDTAVYFCARDAMEGNSYFDHWGPGTLVTVSS

SEQ ID NO: 50 - protein, 5 aa, synthetic construct
RYYLH

SEQ ID NO: 51 - protein, 17 aa, synthetic construct
VINPYGGATNYAQKFQG

SEQ ID NO: 52 - protein, 11 aa, synthetic construct
DAMEGNSYFDH

SEQ ID NO: 53 - protein, 120 aa, synthetic construct
QVQLVQSGAEVKKPGASMTVSCKTSGYTFISYYIHWLRQAPGQGLEWLGVINPFGGATNYAQKFQGRVTMTRDTSTITVYMELSSLRSEDTAVYYCARDAMGGDSHFDFWGQGTLVTVSS

SEQ ID NO: 54 - protein, 5 aa, synthetic construct
SYYIH

SEQ ID NO: 55 - protein, 17 aa, synthetic construct
VINPFGGATNYAQKFQG

SEQ ID NO: 56 - protein, 11 aa, synthetic construct
DAMGGDSHFDF

SEQ ID NO: 57 - protein, 120 aa, synthetic construct
QVQLVESGTEVKKPGASVKVSCKASGYSFIHYYIHWVRQAPGRGLEWMGVINPFDGSTNYAQNFQGRVTVTRDTSTSTVFMDLSSLRSEDTAVYYCARDNMIGYVLFDHWGQGTLVTVSS

SEQ ID NO: 58 - protein, 5 aa, synthetic construct
HYYIH

SEQ ID NO: 59 - skipped

SEQ ID NO: 60 - protein, 17 aa, synthetic construct
VINPFDGSTNYAQNFQG

SEQ ID NO: 61 - protein, 11 aa, synthetic construct
DNMIGYVLFDH

SEQ ID NO: 62 - protein, 120 aa, synthetic construct
QVQLVESGAEVKKPGASVKVSCKASGYSFTSYYIQWVRQAPGQGLEWLAVINPIDGSTNYAQKFQGRVTVTRDTSTSTVYMELSSLRSEDTAVYYCARDYMRGFVVFDYWGQGTLVTVSS

SEQ ID NO: 63 - protein, 5 aa, synthetic construct
SYYIQ

SEQ ID NO: 64 - protein, 17 aa, synthetic construct
VINPIDGSTNYAQKFQG

SEQ ID NO: 65 - protein, 11 aa, synthetic construct
DYMRGFVVFDY

SEQ ID NO: 66 - protein, 120 aa, synthetic construct
QVQLVQSGAEVKKPGASVRVSCKASGYTFTNYYIHWVRQAPGHGLTWMGVINPFDSSTDYAQKFQGRVTMTRDTSTSTVYMDLSSLRSEDTAVYYCARDYMRGHVVFDYWGQGTLVTVSS

SEQ ID NO: 67 - protein, 5 aa, synthetic construct
NYYIH

SEQ ID NO: 68 - protein, 17 aa, synthetic construct
VINPFDSSTDYAQKFQG

SEQ ID NO: 69 - protein, 11 aa, synthetic construct
DYMRGHVVFDY

SEQ ID NO: 70 - protein, 120 aa, synthetic construct
QVQLVQSGAEVKKPGASVKVSCKASGYAFPNYYIHWVRQAPGQGLEWMGVINPYGGSTNYAQKFQGRVTMTRGTSTSTVYLELSSLRSEDTAVYYCARDAMKGDSFFDYWGQGTLVTVSS

SEQ ID NO: 71 - protein, 5 aa, synthetic construct
NYYIH

SEQ ID NO: 72 - protein, 17 aa, synthetic construct
VINPYGGSTNYAQKFQG

SEQ ID NO: 73 - protein, 11 aa, synthetic construct
DAMKGDSFFDY

SEQ ID NO: 74 - protein, 120 aa, synthetic construct
QVQLVQSGAEVKKPGASVQVSCKASGYTFINYYLQWVRQAPGQGLEWMGTINPFDGSTHYEQNFQGRVTMTRDTSTNTVYMELNGLRSEDTAVYYCARDHMIGYILFDYWGQGTTVTVSS

SEQ ID NO: 75 - protein, 5 aa, synthetic construct
NYYLQ

SEQ ID NO: 76 - protein, 17 aa, synthetic construct
TINPFDGSTHYEQNFQG

SEQ ID NO: 77 - protein, 11 aa, synthetic construct
DHMIGYILFDY

SEQ ID NO: 78 - protein, 120 aa, synthetic construct
QVQLVQSGAEVKKPGTSVKVSCKASGYSFIHYYIHWVRQAPGQGLEWVGVINPFDGSTNYAQKFQGRVTVTRDTSTSAVFMDLSSLRSEDTAVYYCARDNMIGYVLFDYWGQGTLVTVSS

SEQ ID NO: 79 - protein, 5 aa, synthetic construct
HYYIH

SEQ ID NO: 80 - protein, 17 aa, synthetic construct
VINPFDGSTNYAQKFQG

SEQ ID NO: 81 - protein, 11 aa, synthetic construct
DNMIGYVLFDY

SEQ ID NO: 82 - protein, 120 aa, synthetic construct
QVQLVQSGAEVKKPGASVKVSCKASGYTFTSYYIHWVRQAPGQGLEWMGVINPYDSSTNYAQKFQGRVTMTRDTSTSTVFMDLSSLRSEDTAVYYCARDNMIGYVLFDYWGQGTLVTVSS

SEQ ID NO: 83 - protein, 5 aa, synthetic construct
SYYIH

SEQ ID NO: 84 - protein, 17 aa, synthetic construct
VINPYDSSTNYAQKFQG

SEQ ID NO: 85 - protein, 11 aa, synthetic construct
DNMIGYVLFDY

SEQ ID NO: 86 - protein, 120 aa, synthetic construct
QVQLVQSGAEVKKPGASVKVSCKASGYPFINYYIQWVRQAPGQGLEWMGVINPFDGSTNYAQKFQGRVTVTRDTSTSTVFMDLSSLRSEDTAVYYCARDNMIGYVLFDYWGQGTLVTVSS

SEQ ID NO: 87 - protein, 5 aa, synthetic construct
NYYIQ

SEQ ID NO: 88 - protein, 17 aa, synthetic construct
VINPFDGSTNYAQKFQG

SEQ ID NO: 89 - protein, 11 aa, synthetic construct
DNMIGYVLFDY

SEQ ID NO: 90 - protein, 120 aa, synthetic construct
QVQLVQSGAEVKKPGASVKVSCKASGYTFINHYIHWVRQAPGQGLEWMGVINPFDGSTNYAQKFQGRVTVTRDTSTSTVFLDLSSLRSEDTAVYYCARDNMIGYVLFDYWGQGTLVTVSS

SEQ ID NO: 91 - protein, 5 aa, synthetic construct
NHYIH

SEQ ID NO: 92 - protein, 17 aa, synthetic construct
VINPFDGSTNYAQKFQG

SEQ ID NO: 93 - protein, 11 aa, synthetic construct
DNMIGYVLFDY

SEQ ID NO: 94 - protein, 120 aa, synthetic construct
QVQLVQSGAEVKKPGSSVKVSCKASGYTFNSYYIHWVRQAPGQGLTWMGVINPFDGSTDYAQKFQGRVTMTRDTSTSTVYMDLSSLRSEDTAVYYCARDYMRGYVVFDYWGRGTLVTVSS

SEQ ID NO: 95 - protein, 5 aa, synthetic construct
SYYIH

SEQ ID NO: 96 - protein, 17 aa, synthetic construct
VINPFDGSTDYAQKFQG

SEQ ID NO: 97 - protein, 11 aa, synthetic construct
DYMRGYVVFDY

SEQ ID NO: 98 - protein, 120 aa, synthetic construct
QVQLVQSGAEVKKPGASVKVSCKASGYTFTSYYIQWLRQAPGQGLEWMGVINPTGGATNYAQKFQGRVTMTRETSTSMVHMELSSLRSEDTAVYYCARDYMRGYVVFDYWGQGTLVTVSS

SEQ ID NO: 99 - protein, 5 aa, synthetic construct
SYYIQ

SEQ ID NO: 100 - protein, 17 aa, synthetic construct
VINPTGGATNYAQKFQG

SEQ ID NO: 101 - protein, 11 aa, synthetic construct
DYMRGYVVFDY

SEQ ID NO: 102 - protein, 120 aa, synthetic construct
QVQLVQSGAEVKKSGASVKVSCKASGYPFISYYIHWLRQAPGQGLTWMGVINPFDGSTDYAQKFQGRVTVTRDTSTSTVYMDLSSLRADDTAIYYCARDYMRGYVVFDYWGQGTLVTVSS

SEQ ID NO: 103 - protein, 5 aa, synthetic construct
SYYIH

SEQ ID NO: 104 - protein, 17 aa, synthetic construct
VINPFDGSTDYAQKFQG

SEQ ID NO: 105 - protein, 11 aa, synthetic construct
DYMRGYVVFDY

SEQ ID NO: 106 - protein, 120 aa, synthetic construct
QVQLVQSGAEMKKPGASVKISCKASGYTFTNYYIQWVRQAPGQGLEWMGVINPYDGSTNYAQRFQGRVTLTRDTSTTTVYMELSSLRSEDTAVYYCARDYMIGYVVFDFWGQGTLVTVSS

SEQ ID NO: 107 - protein, 5 aa, synthetic construct
NYYIQ

SEQ ID NO: 108 - protein, 17 aa, synthetic construct
VINPYDGSTNYAQRFQG

SEQ ID NO: 109 - protein, 11 aa, synthetic construct
DYMIGYVVFDF

SEQ ID NO: 110 - protein, 120 aa, synthetic construct
QVQLVQSGAEMKKPGASVKVSCKASGYTFTNYYIHWVRQAPGQGLEWMGVINPYDGSTNYAQKFQGRVTLTRDTSTTTVYMELSSLRSEDTAVYYCARDYMIGYVVFDFWGQGTLVTVSS

SEQ ID NO: 111 - protein, 5 aa, synthetic construct
NYYIH

SEQ ID NO: 112 - protein, 17 aa, synthetic construct
VINPYDGSTNYAQKFQG

SEQ ID NO: 113 - protein, 11 aa, synthetic construct
DYMIGYVVFDF

SEQ ID NO: 114 - protein, 120 aa, synthetic construct
QVQLVQSGAEMKKPGASVTVSCKASGYTFTNYYIQWVRQAPGQGLEWMGVINPYDGSTNYAQRFQGRVTLTRDTTTTTVNMELSGLRSEDTAVYYCARDYMIGYVVFDFWGQGTLVTVSS

SEQ ID NO: 115 - protein, 5 aa, synthetic construct
NYYIQ

SEQ ID NO: 116 - protein, 17 aa, synthetic construct
VINPYDGSTNYAQRFQG

SEQ ID NO: 117 - protein, 11 aa, synthetic construct
DYMIGYVVFDF

SEQ ID NO: 118 - protein, 120 aa, synthetic construct
QVQLVQSGAEVKKPGASVKVSCKASGYTFTNYYIQWVRQAPGQGLEWMGVINPYDGSTNYAQRFQGKITLTRDTSTTTVYMELSSLGSEDTAVYYCARDYMIGYVVFDYWGQGTMVTVSS

SEQ ID NO: 119 - protein, 5 aa, synthetic construct
NYYIQ

SEQ ID NO: 120 - protein, 17 aa, synthetic construct
VINPYDGSTNYAQRFQG

SEQ ID NO: 121 - protein, 11 aa, synthetic construct
DYMIGYVVFDY

SEQ ID NO: 122 - protein, 120 aa, synthetic construct
EVQLLESGAEVKKPGASVKVSCKASGYTFNRYYIHWVRQAPGQGLEWMGVINPYDGSTDYAQKFRDRITMTRDTSTNTVYMDLSSLRSEDTAVYFCARDAMEGDSYFDNWGRGTLVTVSS

SEQ ID NO: 123 - protein, 5 aa, synthetic construct
RYYIH

SEQ ID NO: 124 - protein, 17 aa, synthetic construct
VINPYDGSTDYAQKFRD

SEQ ID NO: 125 - protein, 11 aa, synthetic construct
DAMEGDSYFDN

SEQ ID NO: 126 - protein, 120 aa, synthetic construct
QVQLVQSGAEVKKPGASVKVSCKASGYTFNRYYLHWVRQAPGQGLEWMGVINPYDGSTNYAQKFRGRVTMTRDTSTNTVYMELSSLRSEDTAVYFCARDAMEGDSYFDNWGQGTTVTVSS

SEQ ID NO: 127 - protein, 5 aa, synthetic construct
RYYLH

SEQ ID NO: 128 - protein, 17 aa, synthetic construct
VINPYDGSTNYAQKFRG

SEQ ID NO: 129 - protein, 11 aa, synthetic construct
DAMEGDSYFDN

SEQ ID NO: 130 - protein, 120 aa, synthetic construct
QVQLVQSGAEVKKPGASVRVSCKASGYTFINYYLQWVRQAPGQGLEWMGTINPFDGSTHYAQKFQGRVTLTRDTSTSTVYMELNSLRSDDTAVYYCARDYMIGYVLFDYWGQGTLVTVSS

SEQ ID NO: 131 - protein, 5 aa, synthetic construct
NYYLQ

SEQ ID NO: 132 - protein, 17 aa, synthetic construct
TINPFDGSTHYAQKFQG

SEQ ID NO: 133 - protein, 11 aa, synthetic construct
DYMIGYVLFDY

SEQ ID NO: 134 - protein, 120 aa, synthetic construct
QVQLVQSGAEVKKPGASVEVSCKASGYTFINYYIHWVRQAPGQGLEWMGVINPFDGSTNYAQKFQGRVTVTRDTSTSTVFLDLSSLRPEDTAVYYCARDSMIGYVLFDYWGQGTLVTVSS

SEQ ID NO: 135 - protein, 5 aa, synthetic construct
NYYIH

SEQ ID NO: 136 - protein, 17 aa, synthetic construct
VINPFDGSTNYAQKFQG

SEQ ID NO: 137 - protein, 11 aa, synthetic construct
DSMIGYVLFDY

SEQ ID NO: 138 - protein, 120 aa, synthetic construct
QVQLVQSGAEVKKPGASVRVSCKASGYTFINYYLQWVRQAPGQGLEWMGTINPFDGSTHYAQKFQGRVTMTRDTSTSTVYMELSSLRSEDTAVYYCARDSMIGYVLFDYWGQGTLVTVSS

SEQ ID NO: 139 - protein, 5 aa, synthetic construct
NYYLQ

SEQ ID NO: 140 - protein, 17 aa, synthetic construct
TINPFDGSTHYAQKFQG

SEQ ID NO: 141 - protein, 11 aa, synthetic construct
DSMIGYVLFDY

SEQ ID NO: 142 - protein, 120 aa, synthetic construct
QVQLVQSGAEVKKPGASVMLSCKASGYIFTSYYIQWVRQAPGQGLEWMGVVNPYDGSTNYAQKFQGRVSITRDTSTSTVFMELNSLRSEDTAVYYCARDSMLGHVIFDFWGQGTLVTVSS

SEQ ID NO: 143 - protein, 5 aa, synthetic construct
SYYIQ

SEQ ID NO: 144 - protein, 17 aa, synthetic construct
VVNPYDGSTNYAQKFQG

SEQ ID NO: 145 - protein, 11 aa, synthetic construct
DSMLGHVIFDF

SEQ ID NO: 146 - protein, 120 aa, synthetic construct
QVQLVQSGAEVKKPGASVKVSCKASGYAFTSYYIHWVRQAPGQGLEWMGVINPYGGSTNYAQKFQGRVTMTRDTSTRIVYMDLSSLRSEDTAVYYCARDAMEGDSYFDYWGQGTLVTVSS

SEQ ID NO: 147 - protein, 5 aa, synthetic construct
SYYIH

SEQ ID NO: 148 - protein, 17 aa, synthetic construct
VINPYGGSTNYAQKFQG

SEQ ID NO: 149 - protein, 11 aa, synthetic construct
DAMEGDSYFDY

SEQ ID NO: 150 - protein, 120 aa, synthetic construct
QVQLVQSGAEVKTPGASVKVSCKASGYTFTNYYIQWVRQAPGQALEWMGVINPYDGSTHYAQKFQGRVTLTRDTSTTTVYMDLSSLRSEDAAVYYCARDYMLGYVVFDYWGQGTLVTVSS

SEQ ID NO: 151 - protein, 5 aa, synthetic construct
NYYIQ

SEQ ID NO: 152 - protein, 17 aa, synthetic construct
VINPYDGSTHYAQKFQG

SEQ ID NO: 153 - protein, 11 aa, synthetic construct
DYMLGYVVFDY

SEQ ID NO: 154 - protein, 120 aa, synthetic construct
QVQLVQSGAEVKKSGASVKVSCKASGYIFTSYYIQWVRQAPGQGLEWMGVINPFDGSTDYAQKFQGRVTVTRDTSTTTVFMELSSLRSEDTAVYYCARDNMLGYVLFDYWGQGTLVTVSS

SEQ ID NO: 155 - protein, 5 aa, synthetic construct
SYYIQ

SEQ ID NO: 156 - protein, 17 aa, synthetic construct
VINPFDGSTDYAQKFQG

SEQ ID NO: 157 - protein, 11 aa, synthetic construct
DNMLGYVLFDY

SEQ ID NO: 158 - protein, 120 aa, synthetic construct
QVQLVQSGAEVKKPGASVKVSCKASGYTFTSYYMHWVRQAPGQGLEWMGVINPFDGSTNYAQKFQGRVTVTRDTATTTVFMDLSSLRSEDTAVYYCARDNMLGYVLFDYWGQGTLVTVSS

SEQ ID NO: 159 - protein, 5 aa, synthetic construct
SYYMH

SEQ ID NO: 160 - protein, 17 aa, synthetic construct
VINPFDGSTNYAQKFQG

SEQ ID NO: 161 - protein, 11 aa, synthetic construct
DNMLGYVLFDY

SEQ ID NO: 162 - protein, 120 aa, synthetic construct
QVQLVQSGAEVKKPGASVMLSCKASGYIFTSYYIQWVRQAPGQGLEWMGVVNPYDGSTNYAQKFQGRVTITRDTSTSTVFMELSSLRSEDTAVYYCARDNMLGHVIFDFWGQGTLVTVSS

SEQ ID NO: 163 - protein, 5 aa, synthetic construct
SYYIQ

SEQ ID NO: 164 - protein, 17 aa, synthetic construct
VVNPYDGSTNYAQKFQG

SEQ ID NO: 165 - protein, 11 aa, synthetic construct
DNMLGHVIFDF

SEQ ID NO: 166 - protein, 120 aa, synthetic construct
QVQLVESGAEVKKPGASVKVSCKASGYTFNRYYIHWVRQAPGQGLEWMGVINPSDGSTNYAQKFRGRITMTRDTSTNIVYMELSSLRSEDTAVYFCARDAMGGDSYFDNWGQGTLVTVSS

SEQ ID NO: 167 - protein, 5 aa, synthetic construct
RYYIH

SEQ ID NO: 168 - protein, 17 aa, synthetic construct
VINPSDGSTNYAQKFRG

SEQ ID NO: 169 - protein, 11 aa, synthetic construct
DAMGGDSYFDN

SEQ ID NO: 170 - protein, 120 aa, synthetic construct
QVQLVQSGAEVKKPGASVQVSCKASGYTFINYYLQWVRQAPGQGLEWMGTINPFDGSTHYAQKFQGRVTMTRDTSTNTVYMELNSLRSEDTAVYYCARDRMIGYVLFDYWGQGTLVTVSS

SEQ ID NO: 171 - protein, 5 aa, synthetic construct
NYYLQ

SEQ ID NO: 172 - protein, 17 aa, synthetic construct
TINPFDGSTHYAQKFQG

SEQ ID NO: 173 - protein, 11 aa, synthetic construct
DRMIGYVLFDY

SEQ ID NO: 174 - protein, 120 aa, synthetic construct
EVQLVESGAEVKKPGASVKVSCKASGYTFNRYYLHWVRQAPGQGLEWMGVINPYDGSTNYAQKFRGRITMTRDTSTNTVYMELSSLRSEDTAVYFCARDAMEGDSYFDKWGQGTLVTVSS

SEQ ID NO: 175 - protein, 5 aa, synthetic construct
RYYLH

SEQ ID NO: 176 - protein, 17 aa, synthetic construct
VINPYDGSTNYAQKFRG

SEQ ID NO: 177 - protein, 11 aa, synthetic construct
DAMEGDSYFDK

SEQ ID NO: 178 - protein, 120 aa, synthetic construct
QVQLVQSGAEVKKPGASVKVSCKASGYTFNRHYIQWVRQAPGQGLEWMGTINPYDGSTFYAQKFQDRVTMTRDTSTSTVYMDLNSLKSEDTAVYYCARDYMRGHVLFDYWGQGTLVTVSS

SEQ ID NO: 179 - protein, 5 aa, synthetic construct
RHYIQ

SEQ ID NO: 180 - protein, 17 aa, synthetic construct
TINPYDGSTFYAQKFQD

SEQ ID NO: 181 - protein, 11 aa, synthetic construct
DYMRGHVLFDY

SEQ ID NO: 182 - protein, 120 aa, synthetic construct
QVQLVQSGAEVKKPGASVKVSCKASGYSFTYSYIQWVRLAPGRGLEWMGVINPYDGSTDYAQKFQGRVTLTRDTSTTTVYMELSSLTSEDTAVYYCARDFMIGYVVFDYWGQGTLVTVSS

SEQ ID NO: 183 - protein, 5 aa, synthetic construct
YSYIQ

SEQ ID NO: 184 - protein, 17 aa, synthetic construct
VINPYDGSTDYAQKFQG

SEQ ID NO: 185 - protein, 11 aa, synthetic construct
DFMIGYVVFDY

SEQ ID NO: 186 - protein, 120 aa, synthetic construct
QVQLVQSGAEVKKPGASVKVSCKASGYTFNRYYIHWVRQAPGQGLEWMGVINPYDGSTNYAQKFRGRITMTRDTSTNTVYMDLSSLRSEDTAVYFCARDAMEGDSYLDNWGRGTLVTVSS

SEQ ID NO: 187 - protein, 5 aa, synthetic construct
RYYIH

SEQ ID NO: 188 - protein, 17 aa, synthetic construct
VINPYDGSTNYAQKFRG

SEQ ID NO: 189 - protein, 11 aa, synthetic construct
DAMEGDSYLDN

SEQ ID NO: 190 - protein, 120 aa, synthetic construct
QVQLVQSGAEVKKPGASVKVSCKASGYAFTNYYIHWVRQAPGHGLTWMGVINPFDSSTDFAQKFQGRLTMTRDTSTSTVYMDLSSLRSEDTAVYYCARDYMRGHVVFDNWGQGTLVTVSS

SEQ ID NO: 191 - protein, 5 aa, synthetic construct
NYYIH

SEQ ID NO: 192 - protein, 17 aa, synthetic construct
VINPFDSSTDFAQKFQG

SEQ ID NO: 193 - protein, 11 aa, synthetic construct
DYMRGHVVFDN

SEQ ID NO: 194 - protein, 120 aa, synthetic construct
QVQLVQSGAEVKKPGTSVKVSCTASGYTFTNYYIQWVRQAPGQGLEWLGVINPYDGSTDYAQKFQGRVTLTRDTSTTTVYMELSSLRSEDTAVYYCARDYMLGYVVFDFWGQGTLVTVSS

SEQ ID NO: 195 - protein, 5 aa, synthetic construct
NYYIQ

SEQ ID NO: 196 - protein, 17 aa, synthetic construct
VINPYDGSTDYAQKFQG

SEQ ID NO: 197 - protein, 11 aa, synthetic construct
DYMLGYVVFDF

SEQ ID NO: 198 - protein, 120 aa, synthetic construct
QVQLVQSGAEVKKPGASVKVSCKASGYTFINNYIQWVRQAPGQGLEWMGVINPYDGSIHYAQKFQGRVTLTRDTSTTTVYMELSSLRSEDTAVYYCARDYMIGYVVFEYWGQGTLVTVSS

SEQ ID NO: 199 - protein, 5 aa, synthetic construct
NNYIQ

SEQ ID NO: 200 - protein, 17 aa, synthetic construct
VINPYDGSIHYAQKFQG

SEQ ID NO: 201 - protein, 11 aa, synthetic construct
DYMIGYVVFEY

SEQ ID NO: 202 - protein, 120 aa, synthetic construct
QVQLVQSGAEVKNPGASVKFSCKASGYTFINYYLQWLRQAPGQGLEWMGTINPFDGSTSYAQNFQGRVTMTRDTSSTTVYMELNSLKSEDTAVYYCARDSMIGYVLFDSWGQGTLVTVSS

SEQ ID NO: 203 - protein, 5 aa, synthetic construct
NYYLQ

SEQ ID NO: 204 - protein, 17 aa, synthetic construct
TINPFDGSTSYAQNFQG

SEQ ID NO: 205 - protein, 11 aa, synthetic construct
DSMIGYVLFDS

SEQ ID NO: 206 - protein, 120 aa, synthetic construct
QVQLVQSGAEVKKPGASVRVSCKASGYPFINYYIQWVRQAPGQGLEWMGVINPFDGSTNYAQKFQGRVTVTRDTSTSTVFMDLNSLRSEDTAVYYCARDSMIGYVLFDFWGQGTLVTVSS

SEQ ID NO: 207 - protein, 5 aa, synthetic construct
NYYIQ

SEQ ID NO: 208 - protein, 17 aa, synthetic construct
VINPFDGSTNYAQKFQG

SEQ ID NO: 209 - protein, 11 aa, synthetic construct
DSMIGYVLFDF

SEQ ID NO: 210 - protein, 446 aa, synthetic construct
QVQLVQSGAEVKKTGSSVKVSCKASGYTFTDHAIHWVRQAPGQALEWMGHFSPGNTDIKYNDKFKGRVTLTVDRSMSTAYMELSSLRSEDTAMYYCKTSTFFFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 220 aa, synthetic construct
DIVMTQSPDSLAVSLGERATINCKSSQSLLNSGDQKNYLTWYQQKPGQPPKLLIYWASTRESGVPDRFSGSGSGTDFTLTISSLQAEDVAVYYCQNDYSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 447 aa, synthetic construct
EVKLVESGGGLVQPGGSMKLSCVASGFTFSNYWMNWVRQSPEKGLEWVAEIRLKSNNYTTHYAESVKGRFTISRDDSKSSVSLQMNNLRVEDTGIYYCTRHYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 219 aa, synthetic construct
DIVMTQAAFSNPVTLGTSASISCRSSKSLLHSNGITYFFWYLQKPGLSPQLLIYQMSNLASGVPDRFSSSGSGTDFTLRISRVEAEDVGVYYCAQNLELPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 450 aa, synthetic construct
QMQLVQSEAELKKPGASVKVSCKASGYSFTGHYMHWVRQAPGQGLEWMGWIDPVTGGTKYAQNFQGWVTMTRDTSIRTAYLELSRLRSDDTAMYYCAREVTGDRGQFDKWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 214 aa, synthetic construct
QSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 457 aa, synthetic construct
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 219 - protein, 6 aa, synthetic construct
QSISSY

SEQ ID NO: 220 - skipped

SEQ ID NO: 221 - protein, 9 aa, synthetic construct
QQSYSTPPT

SEQ ID NO: 222 - protein, 15 aa, synthetic construct
EPKSCDKTHTCPPCP

SEQ ID NO: 223 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 224 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 225 - protein, 106 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 226 - protein, 107 aa, synthetic construct [sequence omitted]

SEQ ID NO: 227 - protein, 95 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 228 - protein, 107 aa, synthetic construct; identical to SEQ ID NO: 218

SEQ ID NO: 229 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 230 - protein, 6 aa, synthetic construct
QSISSY

SEQ ID NO: 231 - skipped

SEQ ID NO: 232 - protein, 10 aa, synthetic construct
QQSYSTPPIT

SEQ ID NO: 233 - protein, 95 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 234 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 235 - protein, 6 aa, synthetic construct
QSVSSN

SEQ ID NO: 236 - skipped

SEQ ID NO: 237 - protein, 9 aa, synthetic construct
QQYNNWPWT

SEQ ID NO: 238 - protein, 96 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 239 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 240 - protein, 7 aa, synthetic construct
QSVSSSY

SEQ ID NO: 241 - skipped

SEQ ID NO: 242 - protein, 9 aa, synthetic construct
QQYGSSPWT

SEQ ID NO: 243 - protein, 96 aa, synthetic construct
SYVLTQPPSVSVAPGETARITCGGDNIGRKSVYWYQQKSGQAPVLVIYYDSDRPSGIPERFSGSNSGNTATLTISRVEAGDEADYYCQVWDGSSDH

SEQ ID NO: 244 - protein, 108 aa, synthetic construct
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 245 - protein, 6 aa, synthetic construct
NIGRKS

SEQ ID NO: 246 - skipped

SEQ ID NO: 247 - protein, 11 aa, synthetic construct
QVWDGSSDHWV

SEQ ID NO: 248 - protein, 9 aa, synthetic construct
QQSYSTPPT

SEQ ID NO: 249 - protein, 24 aa, synthetic construct
PAPGSTAPPAHGVTSAPDTRPAPG

SEQ ID NO: 250 - protein, 9 aa, synthetic construct
APDTRRPAP

SEQ ID NO: 251 - protein, 20 aa, synthetic construct
HGVTSAPDTRPAPGSTAPPA

SEQ ID NO: 252 - protein, 40 aa, synthetic construct
PAPGSTAPPAHGVTSAPDTRPAPGSTAPPAHGVTSAPDTR

SEQ ID NO: 253 - protein, 40 aa, synthetic construct; identical to SEQ ID NO: 252
  SITE 5: note = modified residue comprising Tn (alpha-GalNAc) glycosylation
  SITE 6: note = modified residue comprising Tn (alpha-GalNAc) glycosylation
  SITE 14: note = modified residue comprising Tn (alpha-GalNAc) glycosylation
  SITE 15: note = modified residue comprising Tn (alpha-GalNAc) glycosylation
  SITE 19: note = modified residue comprising Tn (alpha-GalNAc) glycosylation
  SITE 25: note = modified residue comprising Tn (alpha-GalNAc) glycosylation
  SITE 26: note = modified residue comprising Tn (alpha-GalNAc) glycosylation
  SITE 34: note = modified residue comprising Tn (alpha-GalNAc) glycosylation
  SITE 35: note = modified residue comprising Tn (alpha-GalNAc) glycosylation
  SITE 39: note = modified residue comprising Tn (alpha-GalNAc) glycosylation

SEQ ID NO: 254 - protein, 40 aa, synthetic construct; identical to SEQ ID NO: 252
  SITE 5: note = modified residue comprising STn (Neu5Ac(alpha(2-6)-GalNAc) glycosylation
  SITE 6: note = modified residue comprising STn (Neu5Ac(alpha(2-6)-GalNAc) glycosylation
  SITE 14: note = modified residue comprising STn (Neu5Ac(alpha(2-6)-GalNAc) glycosylation
  SITE 15: note = modified residue comprising STn (Neu5Ac(alpha(2-6)-GalNAc) glycosylation
  SITE 19: note = modified residue comprising STn (Neu5Ac(alpha(2-6)-GalNAc) glycosylation
  SITE 25: note = modified residue comprising STn (Neu5Ac(alpha(2-6)-GalNAc) glycosylation
  SITE 26: note = modified residue comprising STn (Neu5Ac(alpha(2-6)-GalNAc) glycosylation
  SITE 34: note = modified residue comprising STn (Neu5Ac(alpha(2-6)-GalNAc) glycosylation
  SITE 35: note = modified residue comprising STn (Neu5Ac(alpha(2-6)-GalNAc) glycosylation
  SITE 39: note = modified residue comprising STn (Neu5Ac(alpha(2-6)-GalNAc) glycosylation

SEQ ID NO: 255 - protein, 24 aa, synthetic construct
PAPGSTAPPAHGVTSAPDTRPAPG

SEQ ID NO: 256 - protein, 24 aa, synthetic construct
  SITE 5: note = modified residue comprising alpha-GalNAc glycosylation
PAPGSTAPPAHGVTSAPDTRPAPG

SEQ ID NO: 257 - protein, 24 aa, synthetic construct
  SITE 6: note = modified residue comprising alpha-GalNAc glycosylation
PAPGSTAPPAHGVTSAPDTRPAPG

SEQ ID NO: 258 - protein, 24 aa, synthetic construct
  SITE 14: note = modified residue comprising alpha-GalNAc glycosylation
PAPGSTAPPAHGVTSAPDTRPAPG

SEQ ID NO: 259 - protein, 24 aa, synthetic construct
  SITE 15: note = modified residue comprising alpha-GalNAc glycosylation
PAPGSTAPPAHGVTSAPDTRPAPG

SEQ ID NO: 260 - protein, 24 aa, synthetic construct
  SITE 19: note = modified residue comprising alpha-GalNAc glycosylation
PAPGSTAPPAHGVTSAPDTRPAPG

SEQ ID NO: 261 - protein, 24 aa, synthetic construct
  SITE 5: note = modified residue comprising alpha-GalNAc glycosylation
  SITE 6: note = modified residue comprising alpha-GalNAc glycosylation
PAPGSTAPPAHGVTSAPDTRPAPG

SEQ ID NO: 262 - protein, 24 aa, synthetic construct
  SITE 6: note = modified residue comprising alpha-GalNAc glycosylation
  SITE 19: note = modified residue comprising alpha-GalNAc glycosylation
PAPGSTAPPAHGVTSAPDTRPAPG

SEQ ID NO: 263 - protein, 24 aa, synthetic construct
  SITE 14: note = modified residue comprising alpha-GalNAc glycosylation
  SITE 15: note = modified residue comprising alpha-GalNAc glycosylation
PAPGSTAPPAHGVTSAPDTRPAPG

SEQ ID NO: 264 - protein, 24 aa, synthetic construct
  SITE 5: note = modified residue comprising alpha-GalNAc glycosylation
  SITE 6: note = modified residue comprising alpha-GalNAc glycosylation
  SITE 19: note = modified residue comprising alpha-GalNAc glycosylation
PAPGSTAPPAHGVTSAPDTRPAPG

SEQ ID NO: 265 - protein, 24 aa, synthetic construct
  SITE 14: note = modified residue comprising alpha-GalNAc glycosylation
  SITE 19: note = modified residue comprising alpha-GalNAc glycosylation
PAPGSTAPPAHGVTSAPDTRPAPG

SEQ ID NO: 266 - protein, 38 aa, synthetic construct
  SITE 23: note = modified residue comprising alpha-GalNAc glycosylation
  SITE 38: note = C terminal amidation
HYTNPSQDVTVPCPVTSTPPTPSPSTPPTPSPSCCHPR

SEQ ID NO: 267 - protein, 24 aa, synthetic construct
PAPGSTAPPAHGVTSAPDTRPAPG

SEQ ID NO: 268 - protein, 24 aa, synthetic construct
  SITE 5: note = modified residue comprising Neu5Ac(alpha(2-6)-GalNAc glycosylation
PAPGSTAPPAHGVTSAPDTRPAPG

SEQ ID NO: 269 - protein, 24 aa, synthetic construct
  SITE 6: note = modified residue comprising Neu5Ac(alpha(2-6)-GalNAc glycosylation
PAPGSTAPPAHGVTSAPDTRPAPG

SEQ ID NO: 270 - protein, 24 aa, synthetic construct
  SITE 14: note = modified residue comprising Neu5Ac(alpha(2-6)-GalNAc glycosylation
PAPGSTAPPAHGVTSAPDTRPAPG

SEQ ID NO: 271 - protein, 24 aa, synthetic construct
  SITE 15: note = modified residue comprising Neu5Ac(alpha(2-6)-GalNAc glycosylation
PAPGSTAPPAHGVTSAPDTRPAPG

SEQ ID NO: 272 - protein, 24 aa, synthetic construct
  SITE 19: note = modified residue comprising Neu5Ac(alpha(2-6)-GalNAc glycosylation
PAPGSTAPPAHGVTSAPDTRPAPG

SEQ ID NO: 273 - protein, 24 aa, synthetic construct
  SITE 5: note = modified residue comprising Neu5Ac(alpha(2-6)-GalNAc glycosylation
  SITE 6: note = modified residue comprising Neu5Ac(alpha(2-6)-GalNAc glycosylation
PAPGSTAPPAHGVTSAPDTRPAPG

SEQ ID NO: 274 - protein, 24 aa, synthetic construct
  SITE 6: note = modified residue comprising Neu5Ac(alpha(2-6)-GalNAc glycosylation
  SITE 19: note = modified residue comprising Neu5Ac(alpha(2-6)-GalNAc glycosylation
PAPGSTAPPAHGVTSAPDTRPAPG

SEQ ID NO: 275 - protein, 24 aa, synthetic construct
  SITE 14: note = modified residue comprising Neu5Ac(alpha(2-6)-GalNAc glycosylation
  SITE 15: note = modified residue comprising Neu5Ac(alpha(2-6)-GalNAc glycosylation
PAPGSTAPPAHGVTSAPDTRPAPG

SEQ ID NO: 276 - protein, 24 aa, synthetic construct
  SITE 5: note = modified residue comprising Neu5Ac(alpha(2-6)-GalNAc glycosylation
  SITE 6: note = modified residue comprising Neu5Ac(alpha(2-6)-GalNAc glycosylation
  SITE 19: note = modified residue comprising Neu5Ac(alpha(2-6)-GalNAc glycosylation
PAPGSTAPPAHGVTSAPDTRPAPG

SEQ ID NO: 277 - protein, 24 aa, synthetic construct
  SITE 14: note = modified residue comprising Neu5Ac(alpha(2-6)-GalNAc glycosylation
  SITE 19: note = modified residue comprising Neu5Ac(alpha(2-6)-GalNAc glycosylation
PAPGSTAPPAHGVTSAPDTRPAPG

SEQ ID NO: 278 - protein, 38 aa, synthetic construct; identical to SEQ ID NO: 266
  SITE 23: note = modified residue comprising Neu5Ac(alpha(2-6)-GalNAc glycosylation
  SITE 38: note = C-terminal amidation

SEQ ID NO: 279 - protein, 8 aa, synthetic construct
  SITE 5: note = modified residue comprising alpha-GalNAc glycosylation
  SITE 8: note = C-terminal amidation
APGSTAPP

SEQ ID NO: 280 - protein, 10 aa, synthetic construct
  SITE 6: note = modified residue comprising alpha-GalNAc glycosylation
  SITE 10: note = C-terminal amidation
TSAPDTRPAP

(19) United States
(12) Patent Application Publication
(10) Pub. No.: US 2023/0250190 A1
(43) Pub. Date: Aug. 10, 2023

(54) BINDING DOMAINS AGAINST CANCER-ASSOCIATED MUC1

(71) Applicant: Merus N.V., Utrecht (NL)

(72) Inventors: Rinse KLOOSTER, Utrecht (NL); Ivy WIDJAJA, Utrecht (NL); Patrick MAYES, Wilmington, DE (US); Horacio G. NASTRI, West Chester, PA (US); Jing ZHOU, Glenn Mills, PA (US); Vijay GUPTA, Lexington, MA (US); Bindu VARGHESE, Natick, MA (US)

(73) Assignee: Merus N.V., Utrecht (NL)

(21) Appl. No.: 18/084,797

(22) Filed: Dec. 20, 2022

(30) Foreign Application Priority Data
Dec. 21, 2021 (NL) 2030198

(51) Int. Cl. C07K 16/30; A61P 35/00

(52) U.S. Cl. CPC C07K 16/3092; A61P 35/00; C07K 2317/565; C07K 2317/55; C07K 2317/522

(57) ABSTRACT

The present disclosure relates to binding domains that bind cancer-associated MUC1, and binding moieties comprising such binding domains. The present disclosure further relates to the use of such binding domains or binding moieties in the treatment of cancer. The present disclosure also relates to nucleic acid encoding such binding domains or binding moieties, and a vector and cell comprising such nucleic acid.

### TECHNICAL FIELD

The present disclosure relates to the field of binding domains and binding moieties, for example antibodies. In particular it relates to the field of therapeutic antibodies for the treatment of cancer. More particularly, it relates to binding domains that bind cancer-associated MUC1, and binding moieties comprising such binding domains.

### BACKGROUND

Mucin1 or MUC1 is a transmembrane glycoprotein that is typically expressed mainly in glandular or luminal epithelial cells. MUC1 forms a physical barrier for lubrication and protection, and aids in signal transduction (Shimizu M, Yamauchi K. J Biochem. 1982; 91(2):515-24). The extracellular domain of MUC1 can extend up to 200-500 nm from the cell surface and is heavily glycosylated. Its sugar branches are negatively charged and can oligomerize, thereby creating a physical barrier that protects the cell (Nath S, Mukherjee P. Trends Mol Med. 2014; 20(6):332-42).

The MUC1 gene is located on chromosome 1: 155,185,824-155,192,916 reverse strand (GRCh38:CM000663.2) and has 29 transcripts. MUC1 is also known under a number of different aliases such as: Mucin 1; Cell Surface Associated; Transmembrane; PEM; PUM; EMA; H23Ag; Episialin; Cancer Antigen 15-3; ADMCKD(1); CD227; MCKD (1); MCD; Tumor-Associated Epithelial Membrane Antigen; Carcinoma-Associated Mucin; Tumor Associated Epithelial Mucin; Tumor-Associated Mucin. External Ids for MUC1 gene are HGNC: 7508 NCBI; Entrez Gene: 4582; Ensembl: ENSG00000185499; OMIM: 158340 and UniProtKB: P15941.

The MUC1 protein consists of a single polypeptide chain that is cleaved after translation by auto-proteolysis at the sea-urchin sperm protein enterokinase and agrin (SEA) domain into two peptide subunits called MUC1-C and MUC-N, both of which remain associated via a stable non-covalent bond (FIG. 1; Gao T. et al. Biomed Pharmacother. 2020; 132:110888). MUC1-C is composed of a short extracellular region with 58 amino acids, a 28 amino acid hydrophobic transmembrane region, and an intracellular cytoplasmic region with 69 amino acids. MUC1-N is located extracellularly and contains an about 20 amino acid sequence repeat with the number of repeats varying from 20 to 120, designated as variable number tandem repeat (VNTR) (HGVTSAPDTRPAPGSTAPPA). VNTR are rich in serine and threonine residues, and each repeat contains five potential sites for O-glycosylation, which under normal conditions will make MUC1 heavily glycosylated with a normal pattern of O-glycosylation (Bose M, Mukherjee P. Vaccines (Basel). 2020; 8(4):659, Zhou D. et al. Molecules. 2018; 23(6):1326).

During malignant transformation, MUC1 becomes overexpressed, loses its polarity and apical distribution with expression both on the surface and in the cytoplasm of epithelial cells. In normal cells, O-glycosylation proceeds to mature elongated and branched O-glycans, while in cancer cells these processes are disrupted. Aberrant MUC1 is characterized by incomplete glycosylation of the carbohydrate side chain, leading to the formation of new carbohydrate side chains, including the truncated O-glycan known as Tn antigen (GalNAca-O-Serine/Threonine) and its sialylated version Sialyl-Tn antigen (STn antigen), which contains a sialic acid α-2,6 linked to GalNAca-O-Serine/Threonine (FIG. 1; Gao T. et al. Biomed Pharmacother. 2020).

Expression of tumor-associated carbohydrates (TACAs) have been implicated in immunomodulation and inhibition of anticancer immune responses, and expression of sialic acid-containing glycans in tumor cells has been correlated with a metastatic phenotype and poor prognosis in patients (Rodrigues Mantuano N. et al. J Immunother Cancer. 2020; 8(2):e001222, Bhatia R. et al. Cancer Metastasis Rev. 2019; 38(1-2):223-236, Anandkumar A, Devaraj H. Scand J Immunol. 2013; 78(1):1-7, Baldus SE. et al. Tumour Biol. 1998; 19(6):445-53). The Tn and STn antigens are typically expressed at high levels in certain tumor cell histologies, including gastric cancer (Moreira IB. et al. Cells. 2020; 9(2):264), colorectal cancer (Ju T, et al. Cancer Biomark. 2014; 14(1):63-81), pancreatic cancer (Thomas D. et al. J Cell Mol Med. 2019; 23(10):6885-6896), and breast cancer (Cazet A. et al. Breast Cancer Res. 2010; 12(3):204).

Development of therapeutic antibodies against the Tn/STn antigens has been reported. Gatipotuzumab (previously known as PankoMAb-GEX) reportedly shows specific binding to cancer cells with high affinity but is no longer investigated in clinical trials (Danielczyk A. et al. Cancer Immunol Immunother. 2006; 55(11):1337-47). Its safety was reported in a phase I trial (Fiedler W. et al. Eur J Cancer. 2016; 63:55-63); however, gatipotuzumab did not show beneficial effects in a phase IIb trial in advanced ovarian cancer (OC) when applied as a maintenance therapy for relapsed OC as a single agent (Ledermann J. et al. ESMO Open. 2022 February; 7(1):100311. Epub 2021 Dec. 15). Antibody 5E5 is a mouse anti-human MUC1-Tn/STn antibody that reportedly shows specificity towards cancer cells (Sorensen A L. et al. Glycobiology. 2006; 16(2):96-107). 5E5 was used to generate a chimeric antigen receptor (CAR) targeting the TnMUC antigen. This CART-TnMUC1 construct is comprised of a mouse anti-human scFv derived from the monoclonal antibody 5E5 which recognizes the epitope comprising Tn glycan of MUC1 and is being evaluated in clinical trials for the treatment of refractory solid-tumor malignancies.

There thus remains a need for novel therapeutic interventions that selectively target cancer-associated MUC1.

### SUMMARY

In certain embodiments, the present disclosure provides a new pharmaceutical agent for the treatment of human disease, in particular for the treatment of cancer.

In certain embodiments, the present disclosure relates to binding domains that bind a MUC1 peptide, wherein the MUC1 peptide comprises the amino acid sequence PAPGSTAPPAHGVTSAPDTRPAPG as set forth in SEQ ID NO: 249, and wherein the threonine (T) residue at position 14 of the MUC1 peptide comprises α-GalNAc glycosylation or Neu5Ac-α(2-6)-GalNAc glycosylation.

In certain embodiments, the present disclosure relates to binding domains that specifically bind to cancer-associated MUC1, wherein the binding domains bind to an epitope comprising the VTSA motif of the N-terminal domain of MUC1, and wherein the threonine (T) residue is α-GalNAc glycosylated or Neu5Ac-α(2-6)-GalNAc glycosylated.

In certain embodiments, the present disclosure relates to binding domains having binding specificity for cancer-associated MUC1, wherein the binding domains comprise a heavy chain variable region as further defined herein.

In certain embodiments, the present disclosure relates to a binding moiety comprising two binding domains as described herein.

In certain embodiments, the present disclosure relates to a pharmaceutical composition comprising an effective amount of a binding domain, or of a binding moiety, as described herein.

In certain embodiments, the present disclosure relates to a binding domain, a binding moiety, or a pharmaceutical composition, as described herein, for use in therapy.

In certain embodiments, the present disclosure relates to a binding domain, a binding moiety, or a pharmaceutical composition, as described herein, for use in the treatment of cancer.

In certain embodiments, the present disclosure relates to a method for treating a disease, comprising administering an effective amount of a binding domain, a binding moiety, or a pharmaceutical composition, as described herein, to an individual in need thereof.

In certain embodiments, the present disclosure relates to a method for treating cancer, comprising administering an effective amount of a binding domain, a binding moiety, or a pharmaceutical composition, as described herein, to an individual in need thereof.

In certain embodiments, the present disclosure relates to a nucleic acid sequence encoding a heavy chain variable region as defined herein.

In certain embodiments, the present disclosure relates to a cell comprising a nucleic acid sequence encoding a heavy chain variable region as defined herein.

In certain embodiments, the present disclosure relates to a cell producing a binding domain, or a binding moiety, as described herein.

In certain embodiments, the present disclosure relates to a kit comprising a container comprising a binding domain or a binding moiety, as described herein, and optionally instructions for use.

### DETAILED DESCRIPTION

In certain embodiments, the present disclosure provides a new pharmaceutical agent for the diagnosis and treatment of disease, in particular in humans, and in particular for the diagnosis and treatment of cancer. In certain embodiments, the present disclosure provides binding domains that specifically bind to cancer-associated MUC1 and not to MUC1 expressed on non-cancerous cells. In particular, in certain embodiments, the present disclosure provides for the first time a genus of binding domains that specifically bind to cancer-associated MUC1 comprising Tn (α-GalNAc) and/or STn (Neu5Ac-α(2-6)-GalNAc) glycosylation at a certain amino acid residue or certain amino acid residues of the VTSA motif, that is distinct from normally glycosylated MUC1.

In certain embodiments, the present disclosure provides a binding domain that binds a MUC1 peptide, wherein the MUC1 peptide comprises or consists of the amino acid sequence PAPGSTAPPAHGVTSAPDTRPAPG (SEQ ID NO: 249), and wherein the threonine (T) residue at position 14 of the peptide, herein indicated in bold and underlined, comprises α-GalNAc glycosylation or Neu5Ac-α(2-6)-GalNAc glycosylation.

In certain embodiments, the present disclosure provides a binding domain that specifically binds to cancer-associated MUC-1, wherein the binding domain binds to an epitope comprising the VTSA motif of the N-terminal domain of MUC1, and wherein the threonine (T) residue is α-GalNAc glycosylated or Neu5Ac-α(2-6)-GalNAc glycosylated.

In certain embodiments, a binding domain of the present disclosure binds a MUC1 peptide, wherein the MUC1 peptide comprises or consists of the amino acid sequence PAPGSTAPPAHGVT*SAPDTRPAPG (SEQ ID NO: 249), and wherein * represents Tn glycosylation. In certain embodiments, a binding domain of the present disclosure binds a MUC1 peptide, wherein the MUC1 peptide comprises or consists of the amino acid sequence PAPGSTAPPAHGVT*SAPDTRPAPG (SEQ ID NO: 249), and wherein * represents STn glycosylation.

In certain embodiments, a binding domain of the present disclosure comprises, in addition to Tn or STn glycosylation of the threonine residue at position 14 of the MUC1 peptide of SEQ ID NO: 249, Tn or STn glycosylation of the serine (S) residue at position 15 of said peptide. In certain embodiments, a binding domain of the present disclosure binds a MUC1 peptide, wherein the MUC1 peptide comprises or consists of the amino acid sequence PAPGSTAPPAHGVT*S*APDTRPAPG (SEQ ID NO: 249), and wherein * represents Tn glycosylation. In certain embodiments, a binding domain of the present disclosure binds a MUC1 peptide, wherein the MUC1 peptide comprises or consists of the amino acid sequence PAPGSTAPPAHGVT*S*APDTRPAPG (SEQ ID NO: 249), and wherein * represents STn glycosylation. In certain embodiments, a binding domain of the present disclosure binds a MUC1 peptide, wherein the MUC1 peptide comprises or consists of the amino acid sequence PAPGSTAPPAHGVT*S*APDTRPAPG (SEQ ID NO: 249), and wherein * represents Tn glycosylation and * represents STn glycosylation. In certain embodiments, a binding domain of the present disclosure binds a MUC1 peptide, wherein the MUC1 peptide comprises or consists of the amino acid sequence PAPGSTAPPAHGVT*S*APDTRPAPG (SEQ ID NO: 249), and wherein * represents STn glycosylation and * represents Tn glycosylation.

Tn glycosylation refers to the presence of an α-GalNAc group; STn glycosylation refers to a Neu5Ac-α(2-6)-GalNAc group.

In certain embodiments, a binding domain of the present disclosure binds to human MUC1.

A “binding domain” refers to a proteinaceous molecule, or part of a proteinaceous molecule, that binds to a target molecule, as can be determined by binding assays well known to a person of ordinary skill in the art. A binding domain can, for instance, be a heavy chain variable region, a heavy chain variable region paired with or linked to a light chain variable region, such as a scFv, or a Fab. In certain embodiments, a binding domain of the present disclosure is a Fab. A In certain embodiments, a “Fab” means a binding domain comprising a heavy chain variable region and a light chain variable region. In certain embodiments, a “Fab” means a binding domain comprising a heavy chain variable region, a light chain variable region, a CH1 and a CL region.

In certain embodiments, binding domains of the present disclosure specifically bind to cancer-associated MUC1, which means that the binding domains do not bind at detectable levels to MUC1 peptides lacking aberrant glycosylation patterns associated with cancer, or do so only at much greater concentrations, as determined in a glycopeptide array, in particular a glycopeptide array as described herein. Aberrant glycosylation patterns associated with cancer include Tn glycosylation (α-GalNAc) and STn glycosylation (Neu5Ac-α(2-6)-GalNAc). Thus, in certain embodiments of the present disclosure, the binding domains do not, or do not substantially, bind to a MUC1 peptide comprising or consisting of the amino acid sequence as set forth in SEQ ID NO: 249 which does not comprise α-GalNAc glycosylation or Neu5Ac-α(2-6)-GalNAc glycosylation.

In certain embodiments, a binding domain of the present disclosure binds to an epitope comprising the VTSA motif of the N-terminal domain of MUC1. In certain embodiments, a binding domain of the present disclosure does not, or does not substantially, bind to a MUC1 peptide that comprises or consists of the amino acid sequence PAPGS*TAPPAHGVTSAPDTRPAPG (SEQ ID NO: 249), wherein * represents Tn or STn glycosylation. In certain embodiments, a binding domain of the present disclosure does not, or does not substantially, bind to a MUC1 peptide that comprises or consists of the amino acid sequence PAPGS*TAPPAHGVTSAPDTRPAPG (SEQ ID NO: 249), wherein * represents STn glycosylation. In certain embodiments, a binding domain of the present disclosure does not, or does not substantially, bind to a MUC1 peptide that comprises or consists of the amino acid sequence PAPGST*APPAHGVTSAPDTRPAPG (SEQ ID NO: 249), wherein * represents Tn or STn glycosylation. In certain embodiments, a binding domain of the present disclosure binds to a MUC1 peptide, wherein the MUC1 peptide comprises or consists of the amino acid sequence PAPGST*APPAHGVTSAPDTRPAPG (SEQ ID NO: 249), and wherein * represents Tn glycosylation. In certain embodiments, a binding domain of the present disclosure does not bind to a MUC1 peptide, wherein the MUC1 peptide comprises or consists of the amino acid sequence PAPGST*APPAHGVTSAPDTRPAPG (SEQ ID NO: 249), and wherein * represents STn glycosylation. In certain embodiments, a binding domain of the present disclosure does not, or does not substantially, bind to a MUC1 peptide that comprises or consists of the amino acid sequence PAPGSTAPPAHGVTS*APDTRPAPG (SEQ ID NO: 249), wherein * represents Tn or STn glycosylation. In certain embodiments, a binding domain of the present disclosure does not, or does not substantially, bind to a MUC1 peptide that comprises or consists of the amino acid sequence PAPGSTAPPAHGVTS*APDTRPAPG (SEQ ID NO: 249), wherein * represents Tn glycosylation. In certain embodiments, a binding domain of the present disclosure does not, or does not substantially, bind to a MUC1 peptide that comprises or consists of the amino acid sequence PAPGSTAPPAHGVTSAPDT*RPAPG (SEQ ID NO: 249), wherein * represents Tn or STn glycosylation. In certain embodiments, a binding domain of the present disclosure does not, or does not substantially, bind to a MUC1 peptide that comprises or consists of the amino acid sequence PAPGSTAPPAHGVTSAPDT*RPAPG (SEQ ID NO: 249), wherein * represents Tn glycosylation. In certain embodiments, a binding domain of the present disclosure does not, or does not substantially, bind to a MUC1 peptide that comprises or consists of the amino acid sequence PAPGS*T*APPAHGVTSAPDTRPAPG (SEQ ID NO: 249), wherein * represents Tn or STn glycosylation. In certain embodiments, a binding domain of the present disclosure also binds to a MUC1 peptide, wherein the MUC1 peptide comprises or consists of the amino acid sequence PAPGST*APPAHGVTSAPDT*RPAPG (SEQ ID NO: 249), and wherein * represents Tn glycosylation. In certain embodiments, a binding domain of the present disclosure does not, or does not substantially, bind to a MUC1 peptide that comprises or consists of the amino acid sequence PAPGST*APPAHGVTSAPDT*RPAPG (SEQ ID NO: 249), wherein * represents STn glycosylation. In certain embodiments, a binding domain of the present disclosure does not, or does not substantially, bind to a MUC1 peptide that comprises or consists of the amino acid sequence PAPGS*T*APPAHGVTSAPDT*RPAPG (SEQ ID NO: 249), wherein * represents Tn or STn glycosylation. In certain embodiments, a binding domain of the present disclosure also binds to a MUC1 peptide, wherein the MUC1 peptide comprises or consists of the amino acid sequence PAPGSTAPPAHGVT*SAPDT*RPAPG (SEQ ID NO: 249), and wherein * represents Tn or STn glycosylation.

In certain embodiments, a binding domain is considered to bind to a MUC1 peptide if it, in bivalent monospecific IgG antibody format, binds the peptide with at least about 2 fold, or about 2-3 fold, or about 3 fold higher binding signal than a negative isotype control in a glycopeptide array. In certain embodiments, a binding domain is considered to bind to a MUC1 peptide if it, in bivalent monospecific IgG antibody format, binds the peptide with an EC50 (in ug/ml) of at least lower than about 200 or 100 or 50 or 10 or 1 or 0.5, or between about 200-0.001 or 100-0.001 or 50-0.001 or 10-0.001 or 1-0.001 or 0.5-0.001 or 200-0.05 or 100-0.05 or 50-0.05 or 10-0.05 or 1-0.05 or 0.5-0.05, in a glycopeptide array. In certain embodiments, a binding domain of the present disclosure binds to a MUC1 peptide if it, in bivalent monospecific IgG antibody format, binds the peptide with at least 2 fold, or 2-3 fold, or 3 fold higher binding signal than a negative isotype control in a glycopeptide array. In certain embodiments, a binding domain is considered to bind to a MUC1 peptide if it, in bivalent monospecific IgG antibody format, binds the peptide with an EC50 (in ug/ml) of at least lower than 200 or 100 or 50 or 10 or 1 or 0.5, or between 200-0.001 or 100-0.001 or 50-0.001 or 10-0.001 or 1-0.001 or 0.5-0.001 or 200-0.05 or 100-0.05 or 50-0.05 or 10-0.05 or 1-0.05 or 0.5-0.05, in a glycopeptide array. In certain embodiments, a binding domain is considered to bind to a MUC1 peptide if it, in bivalent monospecific IgG antibody format, binds the peptide with at least about 2 fold, or about 2-3 fold, or about 3 fold higher binding signal than a negative isotype control in a glycopeptide array, and it has an EC50 (in ug/ml) of at least lower than about 200 or 100 or 50 or 10 or 1 or 0.5, or between about 200-0.001 or 100-0.001 or 50-0.001 or 10-0.001 or 1-0.001 or 0.5-0.001 or 200-0.05 or 100-0.05 or 50-0.05 or 10-0.05 or 1-0.05 or 0.5-0.05 in a glycopeptide array. In certain embodiments, a binding domain of the present disclosure binds to a MUC1 peptide if it, in bivalent monospecific IgG antibody format, binds the peptide with at least 2 fold, or 2-3 fold, or 3 fold higher binding signal than a negative isotype control in a glycopeptide, and it has an EC50 (in ug/ml) of at least lower than 200 or 100 or 50 or 10 or 1 or 0.5, or between 200-0.001 or 100-0.001 or 50-0.001 or 10-0.001 or 1-0.001 or 0.5-0.001 or 200-0.05 or 100-0.05 or 50-0.05 or 10-0.05 or 1-0.05 or 0.5-0.05 in a glycopeptide array.

In certain embodiments, a binding domain is considered to not, or to not substantially, bind to a MUC1 peptide if it, in bivalent monospecific IgG antibody format, binds the peptide with less than about 2 fold, or about 2-3 fold, or about 3 fold higher binding signal than a negative isotype control in a glycopeptide array. In certain embodiments, a binding domain is considered to not, or to not substantially, bind to a MUC1 peptide if it, in bivalent monospecific IgG antibody format, binds the peptide with an EC50 (in ug/ml) of higher than about 200 in a glycopeptide array. In certain embodiments, a binding domain of the present disclosure does not, or does not substantially, bind to a MUC1 peptide if it, in bivalent monospecific IgG antibody format, binds the peptide with less than 2 fold, or 2-3 fold, or 3 fold higher binding signal than a negative isotype control in a glycopeptide array. In certain embodiments, a binding domain is considered to not, or to not substantially, bind to a MUC1 peptide if it, in bivalent monospecific IgG antibody format, binds the peptide with an EC50 (in ug/ml) of higher than 200 in a glycopeptide array. In certain embodiments, a binding domain is considered to not, or to not substantially, bind to a MUC1 peptide if it, in bivalent monospecific IgG antibody format, binds the peptide with less than about 2 fold, or about 2-3 fold, or about 3 fold higher binding signal than a negative isotype control in a glycopeptide array. In certain embodiments, a binding domain is considered to not, or to not substantially, bind to a MUC1 peptide if it, in bivalent monospecific IgG antibody format, binds the peptide with an EC50 (in ug/ml) of higher than about 200 in a glycopeptide array. In certain embodiments, a binding domain is considered to not, or to not substantially, bind to a MUC1 peptide if it, in bivalent monospecific IgG antibody format, binds the peptide with less than about 2 fold, or about 2-3 fold, or about 3 fold higher binding signal than a negative isotype control in a glycopeptide array, and it has an EC50 (in ug/ml) of higher than about 200 in a glycopeptide array. In certain embodiments, a binding domain is considered to not, or to not substantially, bind to a MUC1 peptide if it, in bivalent monospecific IgG antibody format, binds the peptide with less than about 2 fold, or about 2-3 fold, or about 3 fold higher binding signal than a negative isotype control in a glycopeptide array, and it has an EC50 (in ug/ml) of higher than 200 in a glycopeptide array.

For the purpose of the present disclosure, determining if a binding domain binds a MUC1 peptide is done by using a glycopeptide array. The binding data of the binding domains as provided herein is obtained with the glycopeptide array as described in Example 5. Therefore, in certain embodiments, the binding of a binding domain of the present disclosure to a MUC1 peptide, or interaction with the VTSA epitope, as described herein, is determined in a glycopeptide array as described in Example 5.

In brief, the glycopeptide array as described in Example 5 is performed using the O-glycopeptides listed in Table 2. The array is blocked before adding MUC1 IgG's and control antibodies in a range from 10 μg/ml to 0.6 ng/ml. The array is covered with an adhesive film and shaken at room temperature. The array is then washed, and detection antibodies are applied to the array. It is covered from light and shaken for an hour at room temperature, followed by washing steps. The array is read using an Innopsys InnoScan 710 Microarray Scanner and suitable software.

Binding domains, or binding moieties, of the present disclosure bind to cancer-associated MUC1, which means that they do not bind to normally glycosylated MUC1 on non-tumor cells. As used herein, “binding to cancer-associated MUC1” refers to the typical binding capacity of a binding domain, or binding moiety, to cancer-associated MUC1 as present on tumor cells or a cell line engineered to express cancer-associated MUC1. Binding of a binding domain, or binding moiety, to an antigen can be assessed in various ways. For the purpose of the present disclosure, determining if a binding domain, or binding moiety, binds cancer-associated MUC1 is done by incubating the binding domain, or binding moiety, with the cells expressing the cancer-associated MUC1, such as MC38 huMUC1 COSMC KO cells, T47D huGalNAc cells stably expressing human MUC1-STn, and/or T47D WT cells expressing medium levels of MUC-Tn, as described herein. Unbound binding domains, or binding moieties, are removed, and bound binding domains, or binding moieties, are detected by means of for instance a labeled antibody that binds to a constant domain of the bound binding domain, or binding moiety.

Antigen binding can be expressed in terms of specificity and affinity. The specificity determines which antigen or epitope thereof is specifically bound by the binding domain or binding moiety. The affinity is a measure for the strength of binding to a particular antigen or epitope.

In certain embodiments, a binding domain of the present disclosure binds to cancer-associated human MUC1.

In certain embodiments, a binding domain of the present disclosure comprises a heavy chain variable region comprising:

a) heavy chain CDR1 (HCDR1), heavy chain CDR2 (HCDR2), and heavy chain CDR3 (HCDR3), having an amino acid sequence as set forth in SEQ ID NO: 2, SEQ ID NO: 3, and SEQ ID NO: 4, respectively;

b) heavy chain CDR1 (HCDR1), heavy chain CDR2 (HCDR2), and heavy chain CDR3 (HCDR3), having an amino acid sequence as set forth in SEQ ID NO: 6, SEQ ID NO: 7, and SEQ ID NO: 8, respectively;

c) heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively;

d) heavy chain CDR1 (HCDR1), heavy chain CDR2 (HCDR2), and heavy chain CDR3 (HCDR3), having an amino acid sequence as set forth in SEQ ID NO: 14, SEQ ID NO: 15, and SEQ ID NO: 16, respectively;

e) heavy chain CDR1 (HCDR1), heavy chain CDR2 (HCDR2), and heavy chain CDR3 (HCDR3), having an amino acid sequence as set forth in SEQ ID NO: 18, SEQ ID NO: 19, and SEQ ID NO: 20, respectively;

f) heavy chain CDR1 (HCDR1), heavy chain CDR2 (HCDR2), and heavy chain CDR3 (HCDR3), having an amino acid sequence as set forth in SEQ ID NO: 22, SEQ ID NO: 23, and SEQ ID NO: 24, respectively;

g) heavy chain CDR1 (HCDR1), heavy chain CDR2 (HCDR2), and heavy chain CDR3 (HCDR3), having an amino acid sequence as set forth in SEQ ID NO: 26, SEQ ID NO: 27, and SEQ ID NO: 28, respectively;

h) heavy chain CDR1 (HCDR1), heavy chain CDR2 (HCDR2), and heavy chain CDR3 (HCDR3), having an amino acid sequence as set forth in SEQ ID NO: 30, SEQ ID NO: 31, and SEQ ID NO: 32, respectively;

i) heavy chain CDR1 (HCDR1), heavy chain CDR2 (HCDR2), and heavy chain CDR3 (HCDR3), having an amino acid sequence as set forth in SEQ ID NO: 34, SEQ ID NO: 35, and SEQ ID NO: 36, respectively;

j) heavy chain CDR1 (HCDR1), heavy chain CDR2 (HCDR2), and heavy chain CDR3 (HCDR3), having an amino acid sequence as set forth in SEQ ID NO: 38, SEQ ID NO: 39, and SEQ ID NO: 40, respectively;

k) heavy chain CDR1 (HCDR1), heavy chain CDR2 (HCDR2), and heavy chain CDR3 (HCDR3), having an amino acid sequence as set forth in SEQ ID NO: 42, SEQ ID NO: 43, and SEQ ID NO: 44, respectively;

l) heavy chain CDR1 (HCDR1), heavy chain CDR2 (HCDR2), and heavy chain CDR3 (HCDR3), having an amino acid sequence as set forth in SEQ ID NO: 46, SEQ ID NO: 47, and SEQ ID NO: 48, respectively;

m) heavy chain CDR1 (HCDR1), heavy chain CDR2 (HCDR2), and heavy chain CDR3 (HCDR3), having an amino acid sequence as set forth in SEQ ID NO: 50, SEQ ID NO: 51, and SEQ ID NO: 52, respectively;

n) heavy chain CDR1 (HCDR1), heavy chain CDR2 (HCDR2), and heavy chain CDR3 (HCDR3), having an amino acid sequence as set forth in SEQ ID NO: 54, SEQ ID NO: 55, and SEQ ID NO: 56, respectively;

o) heavy chain CDR1 (HCDR1), heavy chain CDR2 (HCDR2), and heavy chain CDR3 (HCDR3), having an amino acid sequence as set forth in SEQ ID NO: 58, SEQ ID NO: 60, and SEQ ID NO: 61, respectively;

p) heavy chain CDR1 (HCDR1), heavy chain CDR2 (HCDR2), and heavy chain CDR3 (HCDR3), having an amino acid sequence as set forth in SEQ ID NO: 63, SEQ ID NO: 64, and SEQ ID NO: 65, respectively;

q) heavy chain CDR1 (HCDR1), heavy chain CDR2 (HCDR2), and heavy chain CDR3 (HCDR3), having an amino acid sequence as set forth in SEQ ID NO: 67, SEQ ID NO: 68, and SEQ ID NO: 69, respectively;

r) heavy chain CDR1 (HCDR1), heavy chain CDR2 (HCDR2), and heavy chain CDR3 (HCDR3), having an amino acid sequence as set forth in SEQ ID NO: 71, SEQ ID NO: 72, and SEQ ID NO: 73, respectively;

s) heavy chain CDR1 (HCDR1), heavy chain CDR2 (HCDR2), and heavy chain CDR3 (HCDR3), having an amino acid sequence as set forth in SEQ ID NO: 75, SEQ ID NO: 76, and SEQ ID NO: 77, respectively;

t) heavy chain CDR1 (HCDR1), heavy chain CDR2 (HCDR2), and heavy chain CDR3 (HCDR3), having an amino acid sequence as set forth in SEQ ID NO: 79, SEQ ID NO: 80, and SEQ ID NO: 81, respectively;

u) heavy chain CDR1 (HCDR1), heavy chain CDR2 (HCDR2), and heavy chain CDR3 (HCDR3), having an amino acid sequence as set forth in SEQ ID NO: 83, SEQ ID NO: 84, and SEQ ID NO: 85, respectively;

v) heavy chain CDR1 (HCDR1), heavy chain CDR2 (HCDR2), and heavy chain CDR3 (HCDR3), having an amino acid sequence as set forth in SEQ ID NO: 87, SEQ ID NO: 88, and SEQ ID NO: 89, respectively;

w) heavy chain CDR1 (HCDR1), heavy chain CDR2 (HCDR2), and heavy chain CDR3 (HCDR3), having an amino acid sequence as set forth in SEQ ID NO: 91, SEQ ID NO: 92, and SEQ ID NO: 93, respectively;

x) heavy chain CDR1 (HCDR1), heavy chain CDR2 (HCDR2), and heavy chain CDR3 (HCDR3), having an amino acid sequence as set forth in SEQ ID NO: 95, SEQ ID NO: 96, and SEQ ID NO: 97, respectively;

y) heavy chain CDR1 (HCDR1), heavy chain CDR2 (HCDR2), and heavy chain CDR3 (HCDR3), having an amino acid sequence as set forth in SEQ ID NO: 99, SEQ ID NO: 100, and SEQ ID NO: 101, respectively;

z) heavy chain CDR1 (HCDR1), heavy chain CDR2 (HCDR2), and heavy chain CDR3 (HCDR3), having an amino acid sequence as set forth in SEQ ID NO: 103, SEQ ID NO: 104, and SEQ ID NO: 105, respectively;

aa) heavy chain CDR1 (HCDR1), heavy chain CDR2 (HCDR2), and heavy chain CDR3 (HCDR3), having an amino acid sequence as set forth in SEQ ID NO: 107, SEQ ID NO: 108, and SEQ ID NO: 109, respectively;

bb) heavy chain CDR1 (HCDR1), heavy chain CDR2 (HCDR2), and heavy chain CDR3 (HCDR3), having an amino acid sequence as set forth in SEQ ID NO: 111, SEQ ID NO: 112, and SEQ ID NO: 113, respectively;

cc) heavy chain CDR1 (HCDR1), heavy chain CDR2 (HCDR2), and heavy chain CDR3 (HCDR3), having an amino acid sequence as set forth in SEQ ID NO: 115, SEQ ID NO: 116, and SEQ ID NO: 117, respectively;

dd) heavy chain CDR1 (HCDR1), heavy chain CDR2 (HCDR2), and heavy chain CDR3 (HCDR3), having an amino acid sequence as set forth in SEQ ID NO: 119, SEQ ID NO: 120, and SEQ ID NO: 121, respectively;

ee) heavy chain CDR1 (HCDR1), heavy chain CDR2 (HCDR2), and heavy chain CDR3 (HCDR3), having an amino acid sequence as set forth in SEQ ID NO: 123, SEQ ID NO: 124, and SEQ ID NO: 125, respectively;

ff) heavy chain CDR1 (HCDR1), heavy chain CDR2 (HCDR2), and heavy chain CDR3 (HCDR3), having an amino acid sequence as set forth in SEQ ID NO: 127, SEQ ID NO: 128, and SEQ ID NO: 129, respectively;

gg) heavy chain CDR1 (HCDR1), heavy chain CDR2 (HCDR2), and heavy chain CDR3 (HCDR3), having an amino acid sequence as set forth in SEQ ID NO: 131, SEQ ID NO: 132, and SEQ ID NO: 133, respectively;

hh) heavy chain CDR1 (HCDR1), heavy chain CDR2 (HCDR2), and heavy chain CDR3 (HCDR3), having an amino acid sequence as set forth in SEQ ID NO: 135, SEQ ID NO: 136, and SEQ ID NO: 137, respectively;

ii) heavy chain CDR1 (HCDR1), heavy chain CDR2 (HCDR2), and heavy chain CDR3 (HCDR3), having an amino acid sequence as set forth in SEQ ID NO: 139, SEQ ID NO: 140, and SEQ ID NO: 141, respectively;

jj) heavy chain CDR1 (HCDR1), heavy chain CDR2 (HCDR2), and heavy chain CDR3 (HCDR3), having an amino acid sequence as set forth in SEQ ID NO: 143, SEQ ID NO: 144, and SEQ ID NO: 145, respectively;

kk) heavy chain CDR1 (HCDR1), heavy chain CDR2 (HCDR2), and heavy chain CDR3 (HCDR3), having an amino acid sequence as set forth in SEQ ID NO: 147, SEQ ID NO: 148, and SEQ ID NO: 149, respectively;

ll) heavy chain CDR1 (HCDR1), heavy chain CDR2 (HCDR2), and heavy chain CDR3 (HCDR3), having an amino acid sequence as set forth in SEQ ID NO: 151, SEQ ID NO: 152, and SEQ ID NO: 153, respectively;

mm) heavy chain CDR1 (HCDR1), heavy chain CDR2 (HCDR2), and heavy chain CDR3 (HCDR3), having an amino acid sequence as set forth in SEQ ID NO: 155, SEQ ID NO: 156, and SEQ ID NO: 157, respectively;

nn) heavy chain CDR1 (HCDR1), heavy chain CDR2 (HCDR2), and heavy chain CDR3 (HCDR3), having an amino acid sequence as set forth in SEQ ID NO: 159, SEQ ID NO: 160, and SEQ ID NO: 161, respectively;

oo) heavy chain CDR1 (HCDR1), heavy chain CDR2 (HCDR2), and heavy chain CDR3 (HCDR3), having an amino acid sequence as set forth in SEQ ID NO: 163, SEQ ID NO: 164, and SEQ ID NO: 165, respectively;

pp) heavy chain CDR1 (HCDR1), heavy chain CDR2 (HCDR2), and heavy chain CDR3 (HCDR3), having an amino acid sequence as set forth in SEQ ID NO: 167, SEQ ID NO: 168, and SEQ ID NO: 169, respectively;

qq) heavy chain CDR1 (HCDR1), heavy chain CDR2 (HCDR2), and heavy chain CDR3 (HCDR3), having an amino acid sequence as set forth in SEQ ID NO: 171, SEQ ID NO: 172, and SEQ ID NO: 173, respectively;

rr) heavy chain CDR1 (HCDR1), heavy chain CDR2 (HCDR2), and heavy chain CDR3 (HCDR3), having an amino acid sequence as set forth in SEQ ID NO: 175, SEQ ID NO: 176, and SEQ ID NO: 177, respectively;

ss) heavy chain CDR1 (HCDR1), heavy chain CDR2 (HCDR2), and heavy chain CDR3 (HCDR3), having an amino acid sequence as set forth in SEQ ID NO: 179, SEQ ID NO: 180, and SEQ ID NO: 181, respectively;

tt) heavy chain CDR1 (HCDR1), heavy chain CDR2 (HCDR2), and heavy chain CDR3 (HCDR3), having an amino acid sequence as set forth in SEQ ID NO: 183, SEQ ID NO: 184, and SEQ ID NO: 185, respectively;

uu) heavy chain CDR1 (HCDR1), heavy chain CDR2 (HCDR2), and heavy chain CDR3 (HCDR3), having an amino acid sequence as set forth in SEQ ID NO: 187, SEQ ID NO: 188, and SEQ ID NO: 189, respectively;

vv) heavy chain CDR1 (HCDR1), heavy chain CDR2 (HCDR2), and heavy chain CDR3 (HCDR3), having an amino acid sequence as set forth in SEQ ID NO: 191, SEQ ID NO: 192, and SEQ ID NO: 193, respectively;

ww) heavy chain CDR1 (HCDR1), heavy chain CDR2 (HCDR2), and heavy chain CDR3 (HCDR3), having an amino acid sequence as set forth in SEQ ID NO: 195, SEQ ID NO: 196, and SEQ ID NO: 197, respectively;

xx) heavy chain CDR1 (HCDR1), heavy chain CDR2 (HCDR2), and heavy chain CDR3 (HCDR3), having an amino acid sequence as set forth in SEQ ID NO: 199, SEQ ID NO: 200, and SEQ ID NO: 201, respectively;

yy) heavy chain CDR1 (HCDR1), heavy chain CDR2 (HCDR2), and heavy chain CDR3 (HCDR3), having an amino acid sequence as set forth in SEQ ID NO: 203, SEQ ID NO: 204, and SEQ ID NO: 205, respectively; or

zz) heavy chain CDR1 (HCDR1), heavy chain CDR2 (HCDR2), and heavy chain CDR3 (HCDR3), having an amino acid sequence as set forth in SEQ ID NO: 207, SEQ ID NO: 208, and SEQ ID NO: 209, respectively.

The heavy chain variable regions of the MUC1 binding domain of the present disclosure may comprise a limited number, such as for instance one, two, or three, non-conservative amino acid substitutions, or an unlimited number of conservative amino acid substitutions.

In certain embodiments, the MUC1 binding domain of the present disclosure also includes MUC1 binding domain variants, wherein each of the HCDRs may comprise at most three, two, or one amino acid variations, for example substitutions. In certain embodiments, only one or two HCDRs may comprise at most three, two, or one amino acid variations, for example substitutions. In certain embodiments, such variants do not comprise amino acid variations in HCDR3. In certain embodiments, the amino acid variation is a conservative amino acid substitution.

Typically, a conservative amino acid substitution involves a variation of an amino acid with a homologous amino acid residue, which is a residue that shares similar characteristics or properties. Homologous amino acids are known in the art, as are routine methods for making amino acid substitutions in antibody binding domains without significantly impacting binding or function of the antibody, see for instance handbooks like Lehninger principles of biochemistry (Nelson D L, Cox M M and Lehninger A L. New York: W.H. Freeman, 2017) or Biochemistry (Berg J M, Tymoczko J L and Stryer L. New York: W.H. Freeman, 2007), incorporated herein in its entirety. In determining whether an amino acid can be replaced with a conserved amino acid, an assessment may typically be made of factors such as, but not limited to, (a) the structure of the polypeptide backbone in the area of the substitution, for example, a sheet or helical conformation, (b) the charge or hydrophobicity of the molecule at the target site, and/or (c) the bulk of the side chain(s). If a residue can be substituted with a residue which has common characteristics, such as a similar side chain or similar charge or hydrophobicity, then such a residue is preferred as a substitute. For example, the following groups can be determined: (1) non-polar: Ala (A), Gly (G), Val (V), Leu (L), Ile (I), Pro (P), Phe (F), Trp (W), Met (M); (2) uncharged polar: Ser (S), Thr (T), Cys (C), Tyr (Y), Asn (N), Gln (Q); (3) acidic: Asp (D), Glu (E); and (4) basic: Lys (K), Arg (R), His (H). Alternatively, the amino acids may be grouped as follows: (1) aromatic: Phe (F), Trp (W), Tyr (Y); (2) apolar: Leu (L), Val (V), Ile (I), Ala (A), Met (M); (3) aliphatic: Ala (A), Val (V), Leu (L), Ile (I); (4) acidic: Asp (D), Glu (E); (5) basic: His (H), Lys (K), Arg (R); and (6) polar: Gln (Q), Asn (N), Ser (S), Thr (T), Tyr (Y).

Alternatively, amino acid residues may be divided into groups based on common side-chain properties: (1) hydrophobic: Met (M), Ala (A), Val (V), Leu (L), Ile (I); (2) neutral hydrophilic: Cys (C), Ser (S), Thr (T), Asn (N), Gln (Q); (3) acidic: Asp (D), Glu (E); (4) basic: His (H), Lys (K), Arg R); (5) residues that influence chain orientation: Gly (G), Pro (P); and (6) aromatic: Trp (W), Tyr (Y), Phe (F).

The substitution of an amino acid residue with another present in the same group would be preferred. Accordingly, conservative amino acid substitution can involve exchanging a member of one of these classes for another member of that same class. Typically, the variation results in no, or substantially no, loss in binding specificity of the binding domain to its intended target.

Additional types of amino acid variations include variations resulting from somatic hypermutation and/or affinity maturation. Binding variants encompassed by the present disclosure include somatically hypermutated or affinity matured heavy chain variable regions, which are heavy chain variable regions derived from the same VH gene segments as the heavy chain variable regions described by sequence herein, the variants having amino acid variations, including non-conservative and/or conservative amino acid substitutions in one, two, or all three HCDRs. Routine methods for affinity maturing antibody binding domains are widely known in the art, see for instance Tabasinezhad M. et al. Immunol Lett. 2019; 212:106-113.

Whether amino acid residues within the CDRs and/or framework regions can be substituted, for instance with a conservative amino acid residue, and without, or substantially without, loss in binding specificity, can be determined by methods well known in the art.

Experimental examples include, but are not limited to, for instance, alanine scanning (Cunningham B C, Wells J A. Science. 1989; 244(4908):1081-5), and deep mutational scanning (Araya C L, Fowler D M. Trends Biotechnol. 2011; 29(9):435-42). Computational methods have also been developed that can predict the effect of amino acid variation, such as for instance described in Sruthi C K, Prakash M. PLoS One. 2020; 15(1):e0227621, Choi Y. et al. PLoS One. 2012; 7(10):e46688, and Munro D, Singh M. Bioinformatics. 2020; 36(22-23):5322-9.

In certain embodiments, a MUC1 binding domain of the present disclosure also includes MUC1 binding domain variants, which, in addition to the variations in the HCDRs referred to above, comprise one or more variations in the framework regions. In certain embodiments, a MUC1 binding domain of the present disclosure comprises a heavy chain variable region having an amino acid sequence as set forth in any one of SEQ ID NO: 1; 5; 9; 13; 17; 21; 25; 29; 33; 37; 41; 45; 49; 53; 57; 62; 66; 70; 74; 78; 82; 86; 90; 94; 98; 102; 106; 110; 114; 118; 122; 126; 130; 134; 138; 142; 146; 150; 154; 158; 162; 166; 170; 174; 178; 182; 186; 190; 194; 198; 202; 206, or having at least 80%, or at least 85%, or at least 90%, or at least 95% sequence identity thereto. In certain embodiments, a MUC1 binding domain variant of the present disclosure comprises no variations in the CDR regions but comprises one or more variations in the framework regions. Such variants have at least 80%, or at least 85%, or at least 90%, or at least 95% sequence identity to the sequences disclosed herein, and are expected to retain MUC1 binding specificity. Thus, in certain embodiments, a MUC1 binding domain of the present disclosure comprises:
a heavy chain variable region having at least 80%, or at least 85%, or at least 90%, or at least 95% sequence identity to the amino acid sequence as set forth in SEQ ID NO: 1, which heavy chain variable region comprises a HCDR1 amino acid sequence as set forth in SEQ ID NO: 2; a HCDR2 amino acid sequence as set forth in SEQ ID NO: 3; and a HCDR3 amino acid sequence as set forth in SEQ ID NO: 4;
a heavy chain variable region having at least 80%, or at least 85%, or at least 90%, or at least 95% sequence identity to the amino acid sequence as set forth in SEQ ID NO: 5, which heavy chain variable region comprises a HCDR1 amino acid sequence as set forth in SEQ ID NO: 6; a HCDR2 amino acid sequence as set forth in SEQ ID NO: 7; and a HCDR3 amino acid sequence as set forth in SEQ ID NO: 8;
a heavy chain variable region having at least 80%, or at least 85%, or at least 90%, or at least 95% sequence identity to the amino acid sequence as set forth in SEQ ID NO: 9, which heavy chain variable region comprises a HCDR1 amino acid sequence as set forth in SEQ ID NO: 10; a HCDR2 amino acid sequence as set forth in SEQ ID NO: 11; and a HCDR3 amino acid sequence as set forth in SEQ ID NO: 12;
a heavy chain variable region having at least 80%, or at least 85%, or at least 90%, or at least 95% sequence identity to the amino acid sequence as set forth in SEQ ID NO: 13, which heavy chain variable region comprises a HCDR1 amino acid sequence as set forth in SEQ ID NO: 14; a HCDR2 amino acid sequence as set forth in SEQ ID NO: 15; and a HCDR3 amino acid sequence as set forth in SEQ ID NO: 16;
a heavy chain variable region having at least 80%, or at least 85%, or at least 90%, or at least 95% sequence identity to the amino acid sequence as set forth in SEQ ID NO: 17, which heavy chain variable region comprises a HCDR1 amino acid sequence as set forth in SEQ ID NO: 18; a HCDR2 amino acid sequence as set forth in SEQ ID NO: 19; and a HCDR3 amino acid sequence as set forth in SEQ ID NO: 20;
a heavy chain variable region having at least 80%, or at least 85%, or at least 90%, or at least 95% sequence identity to the amino acid sequence as set forth in SEQ ID NO: 21, which heavy chain variable region comprises a HCDR1 amino acid sequence as set forth in SEQ ID NO: 22; a HCDR2 amino acid sequence as set forth in SEQ ID NO: 23; and a HCDR3 amino acid sequence as set forth in SEQ ID NO: 24;
a heavy chain variable region having at least 80%, or at least 85%, or at least 90%, or at least 95% sequence identity to the amino acid sequence as set forth in SEQ ID NO: 25, which heavy chain variable region comprises a HCDR1 amino acid sequence as set forth in SEQ ID NO: 26; a HCDR2 amino acid sequence as set forth in SEQ ID NO: 27; and a HCDR3 amino acid sequence as set forth in SEQ ID NO: 28;
a heavy chain variable region having at least 80%, or at least 85%, or at least 90%, or at least 95% sequence identity to the amino acid sequence as set forth in SEQ ID NO: 29, which heavy chain variable region comprises a HCDR1 amino acid sequence as set forth in SEQ ID NO: 30; a HCDR2 amino acid sequence as set forth in SEQ ID NO: 31; and a HCDR3 amino acid sequence as set forth in SEQ ID NO: 32;
a heavy chain variable region having at least 80%, or at least 85%, or at least 90%, or at least 95% sequence identity to the amino acid sequence as set forth in SEQ ID NO: 33, which heavy chain variable region comprises a HCDR1 amino acid sequence as set forth in SEQ ID NO: 34; a HCDR2 amino acid sequence as set forth in SEQ ID NO: 35; and a HCDR3 amino acid sequence as set forth in SEQ ID NO: 36;
a heavy chain variable region having at least 80%, or at least 85%, or at least 90%, or at least 95% sequence identity to the amino acid sequence as set forth in SEQ ID NO: 37, which heavy chain variable region comprises a HCDR1 amino acid sequence as set forth in SEQ ID NO: 38; a HCDR2 amino acid sequence as set forth in SEQ ID NO: 39; and a HCDR3 amino acid sequence as set forth in SEQ ID NO: 40;
a heavy chain variable region having at least 80%, or at least 85%, or at least 90%, or at least 95% sequence identity to the amino acid sequence as set forth in SEQ ID NO: 41, which heavy chain variable region comprises a HCDR1 amino acid sequence as set forth in SEQ ID NO: 42; a HCDR2 amino acid sequence as set forth in SEQ ID NO: 43; and a HCDR3 amino acid sequence as set forth in SEQ ID NO: 44;
a heavy chain variable region having at least 80%, or at least 85%, or at least 90%, or at least 95% sequence identity to the amino acid sequence as set forth in SEQ ID NO: 45, which heavy chain variable region comprises a HCDR1 amino acid sequence as set forth in SEQ ID NO: 46; a HCDR2 amino acid sequence as set forth in SEQ ID NO: 47; and a HCDR3 amino acid sequence as set forth in SEQ ID NO: 48;
a heavy chain variable region having at least 80%, or at least 85%, or at least 90%, or at least 95% sequence identity to the amino acid sequence as set forth in SEQ ID NO: 49, which heavy chain variable region comprises a HCDR1 amino acid sequence as set forth in SEQ ID NO: 50; a HCDR2 amino acid sequence as set forth in SEQ ID NO: 51; and a HCDR3 amino acid sequence as set forth in SEQ ID NO: 52;
a heavy chain variable region having at least 80%, or at least 85%, or at least 90%, or at least 95% sequence identity to the amino acid sequence as set forth in SEQ ID NO: 53, which heavy chain variable region comprises a HCDR1 amino acid sequence as set forth in SEQ ID NO: 54; a HCDR2 amino acid sequence as set forth in SEQ ID NO: 55; and a HCDR3 amino acid sequence as set forth in SEQ ID NO: 56;
a heavy chain variable region having at least 80%, or at least 85%, or at least 90%, or at least 95% sequence identity to the amino acid sequence as set forth in SEQ ID NO: 57, which heavy chain variable region comprises a HCDR1 amino acid sequence as set forth in SEQ ID NO: 58; a HCDR2 amino acid sequence as set forth in SEQ ID NO: 60; and a HCDR3 amino acid sequence as set forth in SEQ ID NO: 61;
a heavy chain variable region having at least 80%, or at least 85%, or at least 90%, or at least 95% sequence identity to the amino acid sequence as set forth in SEQ ID NO: 62, which heavy chain variable region comprises a HCDR1 amino acid sequence as set forth in SEQ ID NO: 63; a HCDR2 amino acid sequence as set forth in SEQ ID NO: 64; and a HCDR3 amino acid sequence as set forth in SEQ ID NO: 65;
a heavy chain variable region having at least 80%, or at least 85%, or at least 90%, or at least 95% sequence identity to the amino acid sequence as set forth in SEQ ID NO: 66, which heavy chain variable region comprises a HCDR1 amino acid sequence as set forth in SEQ ID NO: 67; a HCDR2 amino acid sequence as set forth in SEQ ID NO: 68; and a HCDR3 amino acid sequence as set forth in SEQ ID NO: 69;
a heavy chain variable region having at least 80%, or at least 85%, or at least 90%, or at least 95% sequence identity to the amino acid sequence as set forth in SEQ ID NO: 70, which heavy chain variable region comprises a HCDR1 amino acid sequence as set forth in SEQ ID NO: 71; a HCDR2 amino acid sequence as set forth in SEQ ID NO: 72; and a HCDR3 amino acid sequence as set forth in SEQ ID NO: 73;
a heavy chain variable region having at least 80%, or at least 85%, or at least 90%, or at least 95% sequence identity to the amino acid sequence as set forth in SEQ ID NO: 74, which heavy chain variable region comprises a HCDR1 amino acid sequence as set forth in SEQ ID NO: 75; a HCDR2 amino acid sequence as set forth in SEQ ID NO: 76; and a HCDR3 amino acid sequence as set forth in SEQ ID NO: 77;
a heavy chain variable region having at least 80%, or at least 85%, or at least 90%, or at least 95% sequence identity to the amino acid sequence as set forth in SEQ ID NO: 78, which heavy chain variable region comprises a HCDR1 amino acid sequence as set forth in SEQ ID NO: 79; a HCDR2 amino acid sequence as set forth in SEQ ID NO: 80; and a HCDR3 amino acid sequence as set forth in SEQ ID NO: 81;
a heavy chain variable region having at least 80%, or at least 85%, or at least 90%, or at least 95% sequence identity to the amino acid sequence as set forth in SEQ ID NO: 82, which heavy chain variable region comprises a HCDR1 amino acid sequence as set forth in SEQ ID NO: 83; a HCDR2 amino acid sequence as set forth in SEQ ID NO: 84; and a HCDR3 amino acid sequence as set forth in SEQ ID NO: 85;
a heavy chain variable region having at least 80%, or at least 85%, or at least 90%, or at least 95% sequence identity to the amino acid sequence as set forth in SEQ ID NO: 86, which heavy chain variable region comprises a HCDR1 amino acid sequence as set forth in SEQ ID NO: 87; a HCDR2 amino acid sequence as set forth in SEQ ID NO: 88; and a HCDR3 amino acid sequence as set forth in SEQ ID NO: 89;
a heavy chain variable region having at least 80%, or at least 85%, or at least 90%, or at least 95% sequence identity to the amino acid sequence as set forth in SEQ ID NO: 90, which heavy chain variable region comprises a HCDR1 amino acid sequence as set forth in SEQ ID NO: 91; a HCDR2 amino acid sequence as set forth in SEQ ID NO: 92; and a HCDR3 amino acid sequence as set forth in SEQ ID NO: 93;
a heavy chain variable region having at least 80%, or at least 85%, or at least 90%, or at least 95% sequence identity to the amino acid sequence as set forth in SEQ ID NO: 94, which heavy chain variable region comprises a HCDR1 amino acid sequence as set forth in SEQ ID NO: 95; a HCDR2 amino acid sequence as set forth in SEQ ID NO: 96; and a HCDR3 amino acid sequence as set forth in SEQ ID NO: 97;
a heavy chain variable region having at least 80%, or at least 85%, or at least 90%, or at least 95% sequence identity to the amino acid sequence as set forth in SEQ ID NO: 98, which heavy chain variable region comprises a HCDR1 amino acid sequence as set forth in SEQ ID NO: 99; a HCDR2 amino acid sequence as set forth in SEQ ID NO: 100; and a HCDR3 amino acid sequence as set forth in SEQ ID NO: 101;
a heavy chain variable region having at least 80%, or at least 85%, or at least 90%, or at least 95% sequence identity to the amino acid sequence as set forth in SEQ ID NO: 102, which heavy chain variable region comprises a HCDR1 amino acid sequence as set forth in SEQ ID NO: 103; a HCDR2 amino acid sequence as set forth in SEQ ID NO: 104; and a HCDR3 amino acid sequence as set forth in SEQ ID NO: 105;
a heavy chain variable region having at least 80%, or at least 85%, or at least 90%, or at least 95% sequence identity to the amino acid sequence as set forth in SEQ ID NO: 106, which heavy chain variable region comprises a HCDR1 amino acid sequence as set forth in SEQ ID NO: 107; a HCDR2 amino acid sequence as set forth in SEQ ID NO: 108; and a HCDR3 amino acid sequence as set forth in SEQ ID NO: 109;
a heavy chain variable region having at least 80%, or at least 85%, or at least 90%, or at least 95% sequence identity to the amino acid sequence as set forth in SEQ ID NO: 110, which heavy chain variable region comprises a HCDR1 amino acid sequence as set forth in SEQ ID NO: 111; a HCDR2 amino acid sequence as set forth in SEQ ID NO: 112; and a HCDR3 amino acid sequence as set forth in SEQ ID NO: 113;
a heavy chain variable region having at least 80%, or at least 85%, or at least 90%, or at least 95% sequence identity to the amino acid sequence as set forth in SEQ ID NO: 114, which heavy chain variable region comprises a HCDR1 amino acid sequence as set forth in SEQ ID NO: 115; a HCDR2 amino acid sequence as set forth in SEQ ID NO: 116; and a HCDR3 amino acid sequence as set forth in SEQ ID NO: 117;
a heavy chain variable region having at least 80%, or at least 85%, or at least 90%, or at least 95% sequence identity to the amino acid sequence as set forth in SEQ ID NO: 118, which heavy chain variable region comprises a HCDR1 amino acid sequence as set forth in SEQ ID NO: 119; a HCDR2 amino acid sequence as set forth in SEQ ID NO: 120; and a HCDR3 amino acid sequence as set forth in SEQ ID NO: 121;
a heavy chain variable region having at least 80%, or at least 85%, or at least 90%, or at least 95% sequence identity to the amino acid sequence as set forth in SEQ ID NO: 122, which heavy chain variable region comprises a HCDR1 amino acid sequence as set forth in SEQ ID NO: 123; a HCDR2 amino acid sequence as set forth in SEQ ID NO: 124; and a HCDR3 amino acid sequence as set forth in SEQ ID NO: 125;
a heavy chain variable region having at least 80%, or at least 85%, or at least 90%, or at least 95% sequence identity to the amino acid sequence as set forth in SEQ ID NO: 126, which heavy chain variable region comprises a HCDR1 amino acid sequence as set forth in SEQ ID NO: 127; a HCDR2 amino acid sequence as set forth in SEQ ID NO: 128; and a HCDR3 amino acid sequence as set forth in SEQ ID NO: 129;
a heavy chain variable region having at least 80%, or at least 85%, or at least 90%, or at least 95% sequence identity to the amino acid sequence as set forth in SEQ ID NO: 130, which heavy chain variable region comprises a HCDR1 amino acid sequence as set forth in SEQ ID NO: 131; a HCDR2 amino acid sequence as set forth in SEQ ID NO: 132; and a HCDR3 amino acid sequence as set forth in SEQ ID NO: 133;
a heavy chain variable region having at least 80%, or at least 85%, or at least 90%, or at least 95% sequence identity to the amino acid sequence as set forth in SEQ ID NO: 134, which heavy chain variable region comprises a HCDR1 amino acid sequence as set forth in SEQ ID NO: 135; a HCDR2 amino acid sequence as set forth in SEQ ID NO: 136; and a HCDR3 amino acid sequence as set forth in SEQ ID NO: 137;
a heavy chain variable region having at least 80%, or at least 85%, or at least 90%, or at least 95% sequence identity to the amino acid sequence as set forth in SEQ ID NO: 138, which heavy chain variable region comprises a HCDR1 amino acid sequence as set forth in SEQ ID NO: 139; a HCDR2 amino acid sequence as set forth in SEQ ID NO: 140; and a HCDR3 amino acid sequence as set forth in SEQ ID NO: 141;
a heavy chain variable region having at least 80%, or at least 85%, or at least 90%, or at least 95% sequence identity to the amino acid sequence as set forth in SEQ ID NO: 142, which heavy chain variable region comprises a HCDR1 amino acid sequence as set forth in SEQ ID NO: 143; a HCDR2 amino acid sequence as set forth in SEQ ID NO: 144; and a HCDR3 amino acid sequence as set forth in SEQ ID NO: 145;
a heavy chain variable region having at least 80%, or at least 85%, or at least 90%, or at least 95% sequence identity to the amino acid sequence as set forth in SEQ ID NO: 146, which heavy chain variable region comprises a HCDR1 amino acid sequence as set forth in SEQ ID NO: 147; a HCDR2 amino acid sequence as set forth in SEQ ID NO: 148; and a HCDR3 amino acid sequence as set forth in SEQ ID NO: 149;
a heavy chain variable region having at least 80%, or at least 85%, or at least 90%, or at least 95% sequence identity to the amino acid sequence as set forth in SEQ ID NO: 150, which heavy chain variable region comprises a HCDR1 amino acid sequence as set forth in SEQ ID NO: 151; a HCDR2 amino acid sequence as set forth in SEQ ID NO: 152; and a HCDR3 amino acid sequence as set forth in SEQ ID NO: 153;
a heavy chain variable region having at least 80%, or at least 85%, or at least 90%, or at least 95% sequence identity to the amino acid sequence as set forth in SEQ ID NO: 154, which heavy chain variable region comprises a HCDR1 amino acid sequence as set forth in SEQ ID NO: 155; a HCDR2 amino acid sequence as set forth in SEQ ID NO: 156; and a HCDR3 amino acid sequence as set forth in SEQ ID NO: 157;
a heavy chain variable region having at least 80%, or at least 85%, or at least 90%, or at least 95% sequence identity to the amino acid sequence as set forth in SEQ ID NO: 158, which heavy chain variable region comprises a HCDR1 amino acid sequence as set forth in SEQ ID NO: 159; a HCDR2 amino acid sequence as set forth in SEQ ID NO: 160; and a HCDR3 amino acid sequence as set forth in SEQ ID NO: 161;
a heavy chain variable region having at least 80%, or at least 85%, or at least 90%, or at least 95% sequence identity to the amino acid sequence as set forth in SEQ ID NO: 162, which heavy chain variable region comprises a HCDR1 amino acid sequence as set forth in SEQ ID NO: 163; a HCDR2 amino acid sequence as set forth in SEQ ID NO: 164; and a HCDR3 amino acid sequence as set forth in SEQ ID NO: 165;
a heavy chain variable region having at least 80%, or at least 85%, or at least 90%, or at least 95% sequence identity to the amino acid sequence as set forth in SEQ ID NO: 166, which heavy chain variable region comprises a HCDR1 amino acid sequence as set forth in SEQ ID NO: 167; a HCDR2 amino acid sequence as set forth in SEQ ID NO: 168; and a HCDR3 amino acid sequence as set forth in SEQ ID NO: 169;
a heavy chain variable region having at least 80%, or at least 85%, or at least 90%, or at least 95% sequence identity to the amino acid sequence as set forth in SEQ ID NO: 170, which heavy chain variable region comprises a HCDR1 amino acid sequence as set forth in SEQ ID NO: 171; a HCDR2 amino acid sequence as set forth in SEQ ID NO: 172; and a HCDR3 amino acid sequence as set forth in SEQ ID NO: 173;
a heavy chain variable region having at least 80%, or at least 85%, or at least 90%, or at least 95% sequence identity to the amino acid sequence as set forth in SEQ ID NO: 174, which heavy chain variable region comprises a HCDR1 amino acid sequence as set forth in SEQ ID NO: 175; a HCDR2 amino acid sequence as set forth in SEQ ID NO: 176; and a HCDR3 amino acid sequence as set forth in SEQ ID NO: 177;
a heavy chain variable region having at least 80%, or at least 85%, or at least 90%, or at least 95% sequence identity to the amino acid sequence as set forth in SEQ ID NO: 178, which heavy chain variable region comprises a HCDR1 amino acid sequence as set forth in SEQ ID NO: 179; a HCDR2 amino acid sequence as set forth in SEQ ID NO: 180; and a HCDR3 amino acid sequence as set forth in SEQ ID NO: 181;
a heavy chain variable region having at least 80%, or at least 85%, or at least 90%, or at least 95% sequence identity to the amino acid sequence as set forth in SEQ ID NO: 182, which heavy chain variable region comprises a HCDR1 amino acid sequence as set forth in SEQ ID NO: 183; a HCDR2 amino acid sequence as set forth in SEQ ID NO: 184; and a HCDR3 amino acid sequence as set forth in SEQ ID NO: 185;
a heavy chain variable region having at least 80%, or at least 85%, or at least 90%, or at least 95% sequence identity to the amino acid sequence as set forth in SEQ ID NO: 186, which heavy chain variable region comprises a HCDR1 amino acid sequence as set forth in SEQ ID NO: 187; a HCDR2 amino acid sequence as set forth in SEQ ID NO: 188; and a HCDR3 amino acid sequence as set forth in SEQ ID NO: 189;
a heavy chain variable region having at least 80%, or at least 85%, or at least 90%, or at least 95% sequence identity to the amino acid sequence as set forth in SEQ ID NO: 190, which heavy chain variable region comprises a HCDR1 amino acid sequence as set forth in SEQ ID NO: 191; a HCDR2 amino acid sequence as set forth in SEQ ID NO: 192; and a HCDR3 amino acid sequence as set forth in SEQ ID NO: 193;
a heavy chain variable region having at least 80%, or at least 85%, or at least 90%, or at least 95% sequence identity to the amino acid sequence as set forth in SEQ ID NO: 194, which heavy chain variable region comprises a HCDR1 amino acid sequence as set forth in SEQ ID NO: 195; a HCDR2 amino acid sequence as set forth in SEQ ID NO: 196; and a HCDR3 amino acid sequence as set forth in SEQ ID NO: 197;
a heavy chain variable region having at least 80%, or at least 85%, or at least 90%, or at least 95% sequence identity to the amino acid sequence as set forth in SEQ ID NO: 198, which heavy chain variable region comprises a HCDR1 amino acid sequence as set forth in SEQ ID NO: 199; a HCDR2 amino acid sequence as set forth in SEQ ID NO: 200; and a HCDR3 amino acid sequence as set forth in SEQ ID NO: 201;
a heavy chain variable region having at least 80%, or at least 85%, or at least 90%, or at least 95% sequence identity to the amino acid sequence as set forth in SEQ ID NO: 202, which heavy chain variable region comprises a HCDR1 amino acid sequence as set forth in SEQ ID NO: 203; a HCDR2 amino acid sequence as set forth in SEQ ID NO: 204; and a HCDR3 amino acid sequence as set forth in SEQ ID NO: 205; or
a heavy chain variable region having at least 80%, or at least 85%, or at least 90%, or at least 95% sequence identity to the amino acid sequence as set forth in SEQ ID NO: 206, which heavy chain variable region comprises a HCDR1 amino acid sequence as set forth in SEQ ID NO: 207; a HCDR2 amino acid sequence as set forth in SEQ ID NO: 208; and a HCDR3 amino acid sequence as set forth in SEQ ID NO: 209.

The MUC1 binding domains of the present disclosure have been generated with a common light chain, in particular with a common light chain referred to as VK1-39/JK1. The binding domains of the present disclosure can comprise any suitable light chain, including but not limited to common light chains known in the art. In certain embodiments, the MUC1 binding domain of the present disclosure comprises common light chain VK1-39/JK1, or a variant thereof harboring a limited number, such as for instance one, two, or three, non-conservative amino acid substitutions, or an unlimited number of conservative amino acid substitutions.

In certain embodiments, a MUC1 binding domain of the present disclosure comprises a light chain variable region having an amino acid sequence as set forth in SEQ ID NO: 218, or a variant thereof. In certain embodiments, a MUC1 binding domain of the present disclosure comprises a light chain variable region having an amino acid sequence as set forth in SEQ ID NO: 218, or having at least 80%, or at least 85%, or at least 90%, or at least 95% sequence identity thereto.

In certain embodiments, a MUC1 binding domain of the present disclosure comprises a light chain variable region comprising light chain CDR1 (LCDR1), light chain CDR2 (LCDR2), and light chain CDR3 (LCDR3), having an amino acid sequence as set forth in SEQ ID NO: 219, SEQ ID NO: 220, and SEQ ID NO: 221, respectively. In certain embodiments, the light chain variable region of a MUC1 binding domain of the present disclosure also includes variants thereof, wherein each of the LCDRs may comprise at most three, two, or one amino acid substitutions. An amino acid substitution is for instance a conservative amino acid substitution.

In certain embodiments, a MUC1 binding domain of the present disclosure also includes MUC1 binding domain variants, which, in addition to the variations in the LCDRs referred to above, comprise one or more variations in the framework regions. In certain embodiments, a MUC1 binding domain variant of the present disclosure comprises no variations in the LCDR regions but comprises one or more variations in the framework regions. Such variants have at least 80%, or at least 85%, or at least 90%, or at least 95% sequence identity to the sequences disclosed herein. Thus, in certain embodiments, a MUC1 binding domain of the present disclosure comprises:
a light chain variable region having at least 80%, or at least 85%, or at least 90%, or at least 95% sequence identity to the amino acid sequence as set forth in SEQ ID NO: 218, which light chain variable region comprises a LCDR1 amino acid sequence as set forth in SEQ ID NO: 219; a LCDR2 amino acid sequence as set forth in SEQ ID NO: 220; and a LCDR3 amino acid sequence as set forth in SEQ ID NO: 221.

A light chain or light chain variable region comprising these LCDRs and/or light chain variable region can be the light chain referred to in the art as VK1-39/JK1. This is a common light chain. The term ‘common light chain’ according to the present disclosure refers to a light chain that is capable of pairing with multiple different heavy chains, such as for instance heavy chains having different antigen or epitope binding specificities. The term “common light chain” encompasses light chains that are identical or have some amino acid sequence differences while the binding specificity of the full length antibody is not affected. It is for instance possible within the scope of the definition of common light chains as used herein, to prepare or find light chains that are not identical but still functionally equivalent, e.g., by using well established variations that introduce conservative amino acid changes, changes of amino acids in regions that are known to or are shown to not or only partly contribute to binding specificity when paired with the heavy chain, and the like.

Apart from a common light chain comprising the LCDRs and/or light chain variable region referred to above, other common light chains known in the art may be used. Examples of such common light chains include, but are not limited to: VK1-39/JK5, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 229. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 229, wherein each of the LCDRs may comprise at most three, two, or one amino acid variations, for example substitutions. In certain embodiments, the light chain comprises a light chain variable region having an amino acid sequence as set forth in SEQ ID NO: 229, or having at least 80%, or at least 85%, or at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 230, SEQ ID NO: 231, and SEQ ID NO: 232, respectively; VK3-15/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 234. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 234, wherein each of the LCDRs may comprise at most three, two, or one amino acid variations, for example substitutions. In certain embodiments, the light chain comprises a light chain variable region having an amino acid sequence as set forth in SEQ ID NO: 234, or having at least 80%, or at least 85%, or at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 235, SEQ ID NO: 236, and SEQ ID NO: 237, respectively; VK3-20/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 239. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 239, wherein each of the LCDRs may comprise at most three, two, or one amino acid variations, for example substitutions. In certain embodiments, the light chain comprises a light chain variable region having an amino acid sequence as set forth in SEQ ID NO: 239, or having at least 80%, or at least 85%, or at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 240, SEQ ID NO: 241, and SEQ ID NO: 242, respectively; and VL3-21/JL3, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 244. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 244, wherein each of the LCDRs may comprise at most three, two, or one amino acid variations, for example substitutions. In certain embodiments, the light chain comprises a light chain variable region having an amino acid sequence as set forth in SEQ ID NO: 244, or having at least 80%, or at least 85%, or at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 245, SEQ ID NO: 246, and SEQ ID NO: 247, respectively.

VK1-39 is short for Immunoglobulin Variable Kappa 1-39 Gene. The gene is also known as Immunoglobulin Kappa Variable 1-39; IGKV139; IGKV1-39; IgVκ1-39. External Ids for the gene are HGNC: 5740; Entrez Gene: 28930; Ensembl: ENSG00000242371. An amino acid sequence for VK1-39 is given as SEQ ID NO: 227. This is the sequence of the V-region. The V-region can be combined with one of five J-regions. Suitable VJ region sequences are indicated as VK1-39/JK1 (SEQ ID NO: 228) and VK1-39/JK5 (SEQ ID NO: 229); alternative names are IgVκ1-39*01/IGJκ1*01 or IgVκ1-39*01/IGJκ5*01 (nomenclature according to the IMGT database worldwide web at imgt.org). These names are exemplary and encompass allelic variants of the gene segments.

VK3-15 is short for Immunoglobulin Variable Kappa 3-15 Gene. The gene is also known as Immunoglobulin Kappa Variable 3-15; IGKV315; IGKV3-15; IgVκ3-15. External Ids for the gene are HGNC: 5816; Entrez Gene: 28913; Ensembl: ENSG00000244437. An amino acid sequence for VK3-15 is given as SEQ ID NO: 233. This is the sequence of the V-region. The V-region can be combined with one of five J-regions. A suitable VJ region sequence is indicated as VK3-15/JK1 (SEQ ID NO: 234); alternative name is Vκ3-15*01/IGJκ1*01 (nomenclature according to the IMGT database worldwide web at imgt.org). This name is exemplary and encompasses allelic variants of the gene segments.

VK3-20 is short for Immunoglobulin Variable Kappa 3-20 Gene. The gene is also known as Immunoglobulin Kappa Variable 3-20; IGKV320; IGKV3-20; IgVκ3-20. External Ids for the gene are HGNC: 5817; Entrez Gene: 28912; Ensembl: ENSG00000239951. An amino acid sequence for VK3-20 is as SEQ ID NO: 238. This is the sequence of the V-region. The V-region can be combined with one of five J-regions. A suitable VJ region sequence is indicated as VK3-20/JK1 (SEQ ID NO: 239); alternative name is IgVκ3-20*01/IGJκ1*01 (nomenclature according to the IMGT database worldwide web at imgt.org). This name is exemplary and encompasses allelic variants of the gene segments.

VL3-21 is short for Immunoglobulin Variable Lambda 3-21 Gene. The gene is also known as Immunoglobulin Lambda Variable 3-21; IGLV321; IGLV3-21; IgVλ3-21. External Ids for the gene are HGNC: 5905; Entrez Gene: 28796; Ensembl: ENSG00000211662.2. An amino acid sequence for VL3-21 is given as SEQ ID NO: 243. This is the sequence of the V-region. The V-region can be combined with one of five J-regions. A suitable VJ region sequence is indicated as VL3-21/JL3 (SEQ ID NO: 244); alternative name is IgVλ3-21/IGJλ3 (nomenclature according to the IMGT database worldwide web at imgt.org). This name is exemplary and encompasses allelic variants of the gene segments.

Further, any light chain variable region of a MUC1 antibody available in the art may be used, or any other light chain variable region that can readily be obtained, such as from, for instance, an antibody display library by showing antigen binding activity when paired with a MUC1 binding domain of the present disclosure.

In certain embodiments, a MUC1 binding domain of a binding moiety of the present disclosure may further comprise a CH1 and CL region. Any CH1 domain may be used, in particular a human CH1 domain. An example of a suitable CH1 domain is provided by the amino acid sequence provided as SEQ ID NO: 223. Any CL domain may be used, in particular a human CL. An example of a suitable CL domain is provided by the amino acid sequence provided as SEQ ID NO: 226.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 2, SEQ ID NO: 3, and SEQ ID NO: 4, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 6, SEQ ID NO: 7, and SEQ ID NO: 8, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 10, SEQ ID NO: 11, and SEQ ID NO: 12, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 14, SEQ ID NO: 15, and SEQ ID NO: 16, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 18, SEQ ID NO: 19, and SEQ ID NO: 20, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variation.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 22, SEQ ID NO: 23, and SEQ ID NO: 24, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 26, SEQ ID NO: 27, and SEQ ID NO: 28, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitution. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 30, SEQ ID NO: 31, and SEQ ID NO: 32, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 34, SEQ ID NO: 35, and SEQ ID NO: 36, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 38, SEQ ID NO: 39, and SEQ ID NO: 40, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 42, SEQ ID NO: 43, and SEQ ID NO: 44, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 46, SEQ ID NO: 47, and SEQ ID NO: 48, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 50, SEQ ID NO: 51, and SEQ ID NO: 52, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 54, SEQ ID NO: 55, and SEQ ID NO: 56, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 58, SEQ ID NO: 60, and SEQ ID NO: 61, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variation.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 63, SEQ ID NO: 64, and SEQ ID NO: 65, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 67, SEQ ID NO: 68, and SEQ ID NO: 69, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 71, SEQ ID NO: 72, and SEQ ID NO: 73, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 75, SEQ ID NO: 76, and SEQ ID NO: 77, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 79, SEQ ID NO: 80, and SEQ ID NO: 81, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 83, SEQ ID NO: 84, and SEQ ID NO: 85, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 87, SEQ ID NO: 88, and SEQ ID NO: 89, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 91, SEQ ID NO: 92, and SEQ ID NO: 93, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 95, SEQ ID NO: 96, and SEQ ID NO: 97, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 99, SEQ ID NO: 100, and SEQ ID NO: 101, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 103, SEQ ID NO: 104, and SEQ ID NO: 105, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 107, SEQ ID NO: 108, and SEQ ID NO: 109, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 111, SEQ ID NO: 112, and SEQ ID NO: 113, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 115, SEQ ID NO: 116, and SEQ ID NO: 117, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 119, SEQ ID NO: 120, and SEQ ID NO: 121, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 123, SEQ ID NO: 124, and SEQ ID NO: 125, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 127, SEQ ID NO: 128, and SEQ ID NO: 129, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 131, SEQ ID NO: 132, and SEQ ID NO: 133, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 135, SEQ ID NO: 136, and SEQ ID NO: 137, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 139, SEQ ID NO: 140, and SEQ ID NO: 141, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 143, SEQ ID NO: 144, and SEQ ID NO: 145, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 147, SEQ ID NO: 148, and SEQ ID NO: 149, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 151, SEQ ID NO: 152, and SEQ ID NO: 153, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 155, SEQ ID NO: 156, and SEQ ID NO: 157, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 159, SEQ ID NO: 160, and SEQ ID NO: 161, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 163, SEQ ID NO: 164, and SEQ ID NO: 165, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 167, SEQ ID NO: 168, and SEQ ID NO: 169, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 171, SEQ ID NO: 172, and SEQ ID NO: 173, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 175, SEQ ID NO: 176, and SEQ ID NO: 177, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 179, SEQ ID NO: 180, and SEQ ID NO: 181, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 183, SEQ ID NO: 184, and SEQ ID NO: 185, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 187, SEQ ID NO: 188, and SEQ ID NO: 189, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 191, SEQ ID NO: 192, and SEQ ID NO: 193, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 195, SEQ ID NO: 196, and SEQ ID NO: 197, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 199, SEQ ID NO: 200, and SEQ ID NO: 201, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 203, SEQ ID NO: 204, and SEQ ID NO: 205, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain CDR1 (HCDR1), heavy chain CDR2 (HCDR2), and heavy chain CDR3 (HCDR3), having an amino acid sequence as set forth in SEQ ID NO: 207, SEQ ID NO: 208, and SEQ ID NO: 209, respectively; and
a light chain CDR1 (LCDR1) having an amino acid sequence as set forth in SEQ ID NO: 219, light chain CDR2 (LCDR2) having an amino acid sequence as set forth in SEQ ID NO: 220, and light chain CDR3 (LCDR3) having an amino acid sequence as set forth in SEQ ID NO: 221, and

wherein each of the HCDRs and/or LCDRs may comprise at most three, two, or one amino acid variations, for example substitutions. In certain embodiments, the HCDRs and/or LCDRs do not comprise amino acid variations.

In one embodiment, the present disclosure provides a MUC1 binding domain comprising:
a heavy chain variable region having an amino acid sequence as set forth in SEQ ID NO: 1, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 5, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 9, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 13, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 17, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 21, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 25, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 29, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 33, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 37, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 41, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 45, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 49, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 53, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 57, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 62, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 66, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 70, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 74, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 78, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 82, or a heavy chain variable region that is at least 80%, or at least 85%, at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 86, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 90, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 94, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 98, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 102, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 106, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 110, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 114, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 118, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 122, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 126, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 130, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 134, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 138, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 142, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 146, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 150, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 154, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 158, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 162, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 166, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 170, or a heavy chain variable region that is at least 80%, or at least 85%, at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 174, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 178, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 182, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 186, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 190, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 194, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 198, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 202, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

In one embodiment, the present disclosure provides a MUC1 binding domain comprising:
a heavy chain variable region having an amino acid sequence as set forth in SEQ ID NO: 206, or a heavy chain variable region that is at least 80%, or at least 85%, or at least 90%, or at least 95% sequence identity thereto; and
a light chain variable region having an amino acid sequence as set forth in SEQ ID NO: 218, or a light chain variable region that is at least 80%, or at least 85%, or at least 90%, or at least 95% sequence identity thereto. In certain embodiments, each of the heavy chain variable region and light chain variable region comprise HCDRs and LCDRs of, respectively, that do not comprise amino acid variations. In certain embodiments, each of the heavy chain variable region and light chain variable region do not comprise amino acid variations.

“Percent (%) identity” as referring to nucleic acid or amino acid sequences herein is defined as the percentage of residues in a candidate sequence that are identical with the residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal JB. SIAM Review. 1983; 25(2), 201-237). The percent sequence identity between two amino acid sequences or nucleic acid sequences may be determined using the Needleman and Wunsch algorithm for the alignment of two sequences. (Needleman S B, Wunsch C D. J Mol Biol. 1970; 48(3):443-53). The Needleman-Wunsch algorithm has been implemented in the computer program NEEDLE. For the purpose of this invention the NEEDLE program from the EMBOSS package is used to determine percent identity of amino acid and nucleic acid sequences (version 2.8.0, Rice P. et al. Trends Genet. 2000; 16(6):276-7, http://emboss.bioinformatics.nl/). For protein sequences, EBLOSUM62 is used for the substitution matrix. For DNA sequences, DNAFULL is used. The parameters used are a gap-open penalty of 10 and a gap extension penalty of 0.5.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment.

In certain embodiments, the present disclosure also provides a binding moiety comprising a MUC1 binding domain as described herein. In certain embodiments, a binding moiety of the present disclosure comprises two MUC1 binding domains as described herein. In certain embodiments, a binding moiety of the present disclosure consists of two MUC1 binding domains and an Fc region. In certain embodiments, an Fc region comprises a hinge, CH2 and CH3 domains.

A “binding moiety” refers to a proteinaceous molecule comprising a binding domain, and includes for instance all antibody formats available in the art, such as for example a full length IgG antibody, immunoconjugates, diabodies, BiTEs, Fab fragments, scFv, tandem scFv, single domain antibody (like V_{HH} and V_{H}), minibodies, scFab, scFv-zipper, nanobodies, DART molecules, TandAb, Fab-scFv, F(ab)′2, F(ab)′2-scFv2, and intrabodies.

In certain embodiments, a binding moiety of the present disclosure is a monospecific binding moiety, in particular a monospecific antibody. A monospecific antibody according to the present disclosure is an antibody, in any antibody format, that comprises one or more binding domains with specificity for a single target. In certain embodiments, a monospecific binding moiety of the present disclosure may further comprise an Fc region or a part thereof. In certain embodiments, a monospecific binding moiety of the present disclosure is an IgG1 antibody.

The Fc region mediates effector functions of an antibody, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function.

In certain embodiments, a binding moiety of the present disclosure has Fc effector function. In certain embodiments, a binding moiety of the present disclosure has enhanced Fc effector function. In certain embodiments, a binding moiety of the present disclosure exhibits antibody-dependent cell-mediated cytotoxicity (ADCC). A binding moiety, such as an antibody, can be engineered to enhance the ADCC activity (for review, see Kubota T et al. Cancer Sci. 2009; 100(9):1566-72). For instance, ADCC activity of an antibody can be improved when the antibody itself has a low ADCC activity, by slightly modifying the constant region of the antibody (Junttila T T. et al. Cancer Res. 2010; 70(11):4481-9). Changes are sometimes also made to improve storage or production or to remove C-terminal lysines (Kubota T et al. Cancer Sci. 2009; 100(9):1566-72). Another way to improve ADCC activity of an antibody is by enzymatically interfering with the glycosylation pathway resulting in a reduced fucose (von Horsten H H. et al. Glycobiology. 2010; 20(12):1607-18). Alternatively, or additionally, multiple other strategies can be used to achieve ADCC enhancement, for instance including glycoengineering (Kyowa Hakko/Biowa, GlycArt (Roche) and Eureka Therapeutics) and mutagenesis, all of which seek to improve Fc binding to low-affinity activating FcγRIIIa, and/or to reduce binding to the low affinity inhibitory FcγRIIb. In certain embodiments, a binding moiety of the present disclosure exhibits enhanced antibody-dependent cell-mediated cytotoxicity (ADCC). In certain embodiments, a binding moiety of the present disclosure is afucosylated.

In certain embodiments, a binding moiety of the present disclosure has a higher binding signal than a reference antibody. In certain embodiments, the binding signal is as measured in a FACS assay with MUC1-Tn expressing cells or MUC-1-STn expressing cells. In certain embodiments, MUC1-Tn expressing cells are MCF7 or T47D cells, as described herein. In certain embodiments, MUC1-Tn expressing ells are huMUC1 COSMC KO cells, which can be generated by stably transfecting MC38 cells with human MUC1 and knocking-out the C1galt1c1 (COSMC) gene, as described herein. In certain embodiments, MUC1-STn expressing cells are T47D huGalNAc cells, which can be generated by stably transfecting T47D cells with a plasmid encoding ST6GALNAC, as described herein. In certain embodiments, the reference antibody is a bivalent monospecific antibody comprising two heavy chains having an amino acid sequence as set forth in SEQ ID NO: 210 and two light chains having an amino acid sequence as set forth in SEQ ID NO: 211.

In certain embodiments, the present disclosure provides a binding moiety comprising a MUC1 binding domain of the present disclosure, wherein the binding moiety is selected from the group consisting of:
an antibody comprising two heavy chain variable regions having an amino acid sequence as set forth in SEQ ID NO: 1 and a light chain variable region having an amino acid sequence as set forth in SEQ ID NO: 218;
an antibody comprising two heavy chain variable regions having an amino acid sequence as set forth in SEQ ID NO: 5 and a light chain variable region having an amino acid sequence as set forth in SEQ ID NO: 218;
an antibody comprising two heavy chain variable regions having an amino acid sequence as set forth in SEQ ID NO: 9 and a light chain variable region having an amino acid sequence as set forth in SEQ ID NO: 218;
an antibody comprising two heavy chain variable regions having an amino acid sequence as set forth in SEQ ID NO: 13 and a light chain variable region having an amino acid sequence as set forth in SEQ ID NO: 218;
an antibody comprising two heavy chain variable regions having an amino acid sequence as set forth in SEQ ID NO: 17 and a light chain variable region having an amino acid sequence as set forth in SEQ ID NO: 218;
an antibody comprising two heavy chain variable regions having an amino acid sequence as set forth in SEQ ID NO: 21 and a light chain variable region having an amino acid sequence as set forth in SEQ ID NO: 218;
an antibody comprising two heavy chain variable regions having an amino acid sequence as set forth in SEQ ID NO: 25 and a light chain variable region having an amino acid sequence as set forth in SEQ ID NO: 218;
an antibody comprising two heavy chain variable regions having an amino acid sequence as set forth in SEQ ID NO: 29 and a light chain variable region having an amino acid sequence as set forth in SEQ ID NO: 218;
an antibody comprising two heavy chain variable regions having an amino acid sequence as set forth in SEQ ID NO: 33 and a light chain variable region having an amino acid sequence as set forth in SEQ ID NO: 218;
an antibody comprising two heavy chain variable regions having an amino acid sequence as set forth in SEQ ID NO: 37 and a light chain variable region having an amino acid sequence as set forth in SEQ ID NO: 218;
an antibody comprising two heavy chain variable regions having an amino acid sequence as set forth in SEQ ID NO: 41 and a light chain variable region having an amino acid sequence as set forth in SEQ ID NO: 218;
an antibody comprising two heavy chain variable regions having an amino acid sequence as set forth in SEQ ID NO: 45 and a light chain variable region having an amino acid sequence as set forth in SEQ ID NO: 218;
an antibody comprising two heavy chain variable regions having an amino acid sequence as set forth in SEQ ID NO: 49 and a light chain variable region having an amino acid sequence as set forth in SEQ ID NO: 218;
an antibody comprising two heavy chain variable regions having an amino acid sequence as set forth in SEQ ID NO: 53 and a light chain variable region having an amino acid sequence as set forth in SEQ ID NO: 218;
an antibody comprising two heavy chain variable regions having an amino acid sequence as set forth in SEQ ID NO: 57 and a light chain variable region having an amino acid sequence as set forth in SEQ ID NO: 218;
an antibody comprising two heavy chain variable regions having an amino acid sequence as set forth in SEQ ID NO: 62 and a light chain variable region having an amino acid sequence as set forth in SEQ ID NO: 218;
an antibody comprising two heavy chain variable regions having an amino acid sequence as set forth in SEQ ID NO: 66 and a light chain variable region having an amino acid sequence as set forth in SEQ ID NO: 218;
an antibody comprising two heavy chain variable regions having an amino acid sequence as set forth in SEQ ID NO: 70 and a light chain variable region having an amino acid sequence as set forth in SEQ ID NO: 218;
an antibody comprising two heavy chain variable regions having an amino acid sequence as set forth in SEQ ID NO: 74 and a light chain variable region having an amino acid sequence as set forth in SEQ ID NO: 218;
an antibody comprising two heavy chain variable regions having an amino acid sequence as set forth in SEQ ID NO: 78 and a light chain variable region having an amino acid sequence as set forth in SEQ ID NO: 218;
an antibody comprising two heavy chain variable regions having an amino acid sequence as set forth in SEQ ID NO: 82 and a light chain variable region having an amino acid sequence as set forth in SEQ ID NO: 218;
an antibody comprising two heavy chain variable regions having an amino acid sequence as set forth in SEQ ID NO: 86 and a light chain variable region having an amino acid sequence as set forth in SEQ ID NO: 218;
an antibody comprising two heavy chain variable regions having an amino acid sequence as set forth in SEQ ID NO: 90 and a light chain variable region having an amino acid sequence as set forth in SEQ ID NO: 218;
an antibody comprising two heavy chain variable regions having an amino acid sequence as set forth in SEQ ID NO: 94 and a light chain variable region having an amino acid sequence as set forth in SEQ ID NO: 218;
an antibody comprising two heavy chain variable regions having an amino acid sequence as set forth in SEQ ID NO: 98 and a light chain variable region having an amino acid sequence as set forth in SEQ ID NO: 218;
an antibody comprising two heavy chain variable regions having an amino acid sequence as set forth in SEQ ID NO: 102 and a light chain variable region having an amino acid sequence as set forth in SEQ ID NO: 218;
an antibody comprising two heavy chain variable regions having an amino acid sequence as set forth in SEQ ID NO: 106 and a light chain variable region having an amino acid sequence as set forth in SEQ ID NO: 218;
an antibody comprising two heavy chain variable regions having an amino acid sequence as set forth in SEQ ID NO: 110 and a light chain variable region having an amino acid sequence as set forth in SEQ ID NO: 218;
an antibody comprising two heavy chain variable regions having an amino acid sequence as set forth in SEQ ID NO: 114 and a light chain variable region having an amino acid sequence as set forth in SEQ ID NO: 218;
an antibody comprising two heavy chain variable regions having an amino acid sequence as set forth in SEQ ID NO: 118 and a light chain variable region having an amino acid sequence as set forth in SEQ ID NO: 218;
an antibody comprising two heavy chain variable regions having an amino acid sequence as set forth in SEQ ID NO: 122 and a light chain variable region having an amino acid sequence as set forth in SEQ ID NO: 218;
an antibody comprising two heavy chain variable regions having an amino acid sequence as set forth in SEQ ID NO: 126 and a light chain variable region having an amino acid sequence as set forth in SEQ ID NO: 218;
an antibody comprising two heavy chain variable regions having an amino acid sequence as set forth in SEQ ID NO: 130 and a light chain variable region having an amino acid sequence as set forth in SEQ ID NO: 218;
an antibody comprising two heavy chain variable regions having an amino acid sequence as set forth in SEQ ID NO: 134 and a light chain variable region having an amino acid sequence as set forth in SEQ ID NO: 218;
an antibody comprising two heavy chain variable regions having an amino acid sequence as set forth in SEQ ID NO: 138 and a light chain variable region having an amino acid sequence as set forth in SEQ ID NO: 218;
an antibody comprising two heavy chain variable regions having an amino acid sequence as set forth in SEQ ID NO: 142 and a light chain variable region having an amino acid sequence as set forth in SEQ ID NO: 218;
an antibody comprising two heavy chain variable regions having an amino acid sequence as set forth in SEQ ID NO: 146 and a light chain variable region having an amino acid sequence as set forth in SEQ ID NO: 218;
an antibody comprising two heavy chain variable regions having an amino acid sequence as set forth in SEQ ID NO: 150 and a light chain variable region having an amino acid sequence as set forth in SEQ ID NO: 218;
an antibody comprising two heavy chain variable regions having an amino acid sequence as set forth in SEQ ID NO: 154 and a light chain variable region having an amino acid sequence as set forth in SEQ ID NO: 218;
an antibody comprising two heavy chain variable regions having an amino acid sequence as set forth in SEQ ID NO: 158 and a light chain variable region having an amino acid sequence as set forth in SEQ ID NO: 218;
an antibody comprising two heavy chain variable regions having an amino acid sequence as set forth in SEQ ID NO: 162 and a light chain variable region having an amino acid sequence as set forth in SEQ ID NO: 218;
an antibody comprising two heavy chain variable regions having an amino acid sequence as set forth in SEQ ID NO: 166 and a light chain variable region having an amino acid sequence as set forth in SEQ ID NO: 218;
an antibody comprising two heavy chain variable regions having an amino acid sequence as set forth in SEQ ID NO: 170 and a light chain variable region having an amino acid sequence as set forth in SEQ ID NO: 218;
an antibody comprising two heavy chain variable regions having an amino acid sequence as set forth in SEQ ID NO: 174 and a light chain variable region having an amino acid sequence as set forth in SEQ ID NO: 218;
an antibody comprising two heavy chain variable regions having an amino acid sequence as set forth in SEQ ID NO: 178 and a light chain variable region having an amino acid sequence as set forth in SEQ ID NO: 218;
an antibody comprising two heavy chain variable regions having an amino acid sequence as set forth in SEQ ID NO: 182 and a light chain variable region having an amino acid sequence as set forth in SEQ ID NO: 218;
an antibody comprising two heavy chain variable regions having an amino acid sequence as set forth in SEQ ID NO: 186 and a light chain variable region having an amino acid sequence as set forth in SEQ ID NO: 218;
an antibody comprising two heavy chain variable regions having an amino acid sequence as set forth in SEQ ID NO: 190 and a light chain variable region having an amino acid sequence as set forth in SEQ ID NO: 218;
an antibody comprising two heavy chain variable regions having an amino acid sequence as set forth in SEQ ID NO: 194 and a light chain variable region having an amino acid sequence as set forth in SEQ ID NO: 218;
an antibody comprising two heavy chain variable regions having an amino acid sequence as set forth in SEQ ID NO: 198 and a light chain variable region having an amino acid sequence as set forth in SEQ ID NO: 218;
an antibody comprising two heavy chain variable regions having an amino acid sequence as set forth in SEQ ID NO: 202 and a light chain variable region having an amino acid sequence as set forth in SEQ ID NO: 218; and
an antibody comprising two heavy chain variable regions having an amino acid sequence as set forth in SEQ ID NO: 206 and a light chain variable region having an amino acid sequence as set forth in SEQ ID NO: 218,

or a binding moiety that competes with said antibody for binding to a MUC1 peptide comprising the amino acid sequence PAPGSTAPPAHGVT*SAPDTRPAPG (SEQ ID NO: 249), and/or PAPGSTAPPAHGVT*S*APDTRPAPG (SEQ ID NO: 249), wherein * represents Tn or STn glycosylation.

Further provided herein are nucleic acids useful for producing a MUC1 binding domain, or a binding moiety comprising a MUC1 binding domain, of the present disclosure. In certain embodiments, such nucleic acids comprise a nucleic acid sequence encoding the heavy chain variable region of a MUC1 binding domain as described herein. In certain embodiments, a nucleic acid of the present disclosure may further comprise a nucleic acid sequence encoding a CH1 region and optionally a hinge, CH2 and/or CH3 region. In certain embodiments, a nucleic acid of the present disclosure may further comprise at least one nucleic acid sequence encoding a light chain variable region, and optionally a CL region. In certain embodiments, the light chain variable region can be a common light chain variable region as described herein.

Further provided herein is a vector useful for producing a MUC1 binding domain, or a binding moiety comprising a MUC1 binding domain, of the present disclosure. In certain embodiments, such expression vector comprises a nucleic acid sequence encoding the heavy chain variable region of a MUC1 binding domain as described herein. In certain embodiments, a vector of the present disclosure may further comprise a nucleic acid sequence encoding a CH1 region and optionally a hinge, CH2 and/or CH3 region. In certain embodiments, a vector of the present disclosure may further comprise at least one nucleic acid sequence encoding a light chain variable region, and optionally a CL region. In certain embodiments, the light chain variable region can be a common light chain variable region as described herein.

In certain embodiments, the present disclosure also provides a cell comprising a nucleic acid sequence encoding the heavy chain variable region of a MUC1 binding domain as described herein. In certain embodiments, a cell of the present disclosure may further comprise a nucleic acid sequence encoding a CH1 region and optionally a hinge, CH2 and/or CH3 region. In certain embodiments, a cell of the present disclosure may further comprise at least one nucleic acid sequence encoding a light chain variable region, and optionally a CL region. In certain embodiments, the light chain variable region can be a common light chain variable region as described herein.

In certain embodiments, the present disclosure also provides a cell producing a MUC1 binding domain, or a binding moiety comprising a MUC1 binding domain, as described herein. In certain embodiments, such cell can be a recombinant cell, which has been transformed with a vector of the present disclosure. In certain embodiments, a cell of the present disclosure comprises a nucleic acid sequence encoding the heavy chain variable region of a MUC1 binding domain as described herein. In certain embodiments, a cell of the present disclosure further comprises a nucleic acid sequence encoding a CH1 region and optionally a hinge, CH2 and/or CH3 region. In certain embodiments, a cell of the present disclosure further comprises at least one nucleic acid sequence encoding a light chain variable region, in particular a light chain variable region as described herein, and optionally a CL region.

In certain embodiments, the present disclosure provides a pharmaceutical composition comprising an effective amount of a MUC1 binding domain, or a binding moiety comprising a MUC1 binding domain, as described herein, and optionally a pharmaceutically acceptable carrier.

In certain embodiments, the present disclosure provides a MUC1 binding domain, or a binding moiety comprising a MUC1 binding domain, as described herein, and a pharmaceutical composition as described herein, for use in therapy.

In certain embodiments, the present disclosure provides a MUC1 binding domain, or a binding moiety comprising a MUC1 binding domain, as described herein, or the pharmaceutical composition as described herein, for use in the treatment of cancer.

In certain embodiments, the present disclosure provides a method for treating a disease, comprising administering an effective amount of a MUC1 binding domain, or a binding moiety comprising a MUC1 binding domain, as described herein, or the pharmaceutical composition as described herein, to an individual in need thereof.

In certain embodiments, the present disclosure provides a method for treating cancer, comprising administering an effective amount of a MUC1 binding domain, or a binding moiety comprising a MUC1 binding domain, as described herein, or the pharmaceutical composition as described herein, to an individual in need thereof.

As used herein, the terms “individual”, “subject” and “patient” are used interchangeably and refer to a mammal such as a human, mouse, rat, hamster, guinea pig, rabbit, cat, dog, monkey, cow, horse, pig and the like, and in particular to a human having cancer.

The terms “treat,” “treating,” and “treatment,” as used herein, refer to any type of intervention or process performed on or administering an active agent or combination of active agents to a subject with the objective of curing or improving a disease or symptom thereof or which produces a positive therapeutic response. As used herein, “positive therapeutic response” refers to a treatment producing a beneficial effect, e.g. reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease, such as, for example, amelioration of at least one symptom of a disease or disorder, e.g. cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease in the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that treats a disease, such as cancer. In some embodiments, a therapeutic amount is an amount sufficient to delay tumor development. In some embodiments, a therapeutic amount is an amount sufficient to prevent or delay tumor recurrence.

As used herein, an effective amount of the agent or composition is one that, for example, may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

An effective amount may vary according to factors such as the disease state, age, sex, and weight of the individual to be treated, and the ability of the agent or combination of agents to elicit a desired response in the individual, which can be readily evaluated by the ordinarily skilled physician or other health care worker.

An effective amount can be administered to a subject in one or more administrations.

An effective amount can also include an amount that balances any toxic or detrimental effects of the agent or combination of agents and the beneficial effects.

The term “agent” refers to a therapeutically active substance, in the present case a binding domain or binding moiety of the present disclosure, or a pharmaceutical composition of the present disclosure.

As used herein, “to comprise” and its conjugations is used in its non-limiting sense to mean that items following the word are included, but items not specifically mentioned are not excluded.

The articles “a” and “an” are used herein to refer to one or more of the grammatical object of the article. By way of example, “an element” means one or more elements.

A reference herein to a patent document or other matter is not to be taken as an admission that that document or matter was known or that the information it contains was part of the common general knowledge at the priority date of any of the claims.

All patent and literature references cited in the present specification are hereby incorporated by reference in their entirety.

Note that in the present specification, unless stated otherwise, amino acid positions assigned to CDRs and frameworks in a variable region of an antibody or antibody fragment are specified according to Kabat's numbering (see Sequences of Proteins of Immunological Interest (National Institute of Health, Bethesda, Md., 1987 and 1991)). Amino acids in the constant regions are indicated according to the EU numbering system.

Accession numbers are primarily given to provide a further method of identification of a target, the actual sequence of the protein bound may vary, for instance because of a mutation in the encoding gene such as those occurring in some cancers or the like. An antigen binding site of a binding domain or binding moiety of the disclosure can bind the antigen and a variety of variants thereof, such as those expressed by some antigen positive immune or tumor cells. HGNC stands for the HUGO Gene nomenclature committee. The number following the abbreviation is the accession number with which information on the gene and protein encoded by the gene can be retrieved from the HGNC database. Entrez Gene provides the accession number or gene ID with which information on the gene or protein encoded by the gene can be retrieved from the NCBI (National Center for Biotechnology Information) database. Ensembl provides the accession number with which information on the gene or protein encoded by the gene can be obtained from the Ensemble database. Ensembl is a joint project between EMBL-EBI and the Wellcome Trust Sanger Institute to develop a software system which produces and maintains automatic annotation on selected eukaryotic genomes.

When herein reference is made to a gene or a protein, the reference is preferably to the human form of the gene or protein. When herein reference is made to a gene or protein reference is made both to the natural gene or protein and to variant forms of the gene or protein as can be detected in tumors, cancers and the like, or as can be detected in human tumors, cancers and the like.

### BRIEF DESCRIPTION OF THE DRAWINGS

The following naming conventions are used herein as follows. In the Figures, reference bivalent monospecific antibodies are indicated in the format SEQ ID NO: A/B, where SEQ ID NO: A refers to the heavy chain of both binding domains and SEQ ID NO: B refers to the light chain of both binding domains. The bivalent monospecific MUC1 IgG's of the present disclosure exemplified herein comprise the light chain of SEQ ID NO: 217, and the CH1, hinge, CH2 and CH3 region of SEQ ID NO: 223, 222, 224, and 225, respectively, and are indicated in the format SEQ ID NO: A, which refers to the heavy chain variable region of both binding domains.

FIG. 1—Schematic representation of MUC1 structure. A) Structure of normal MUC1; B) Structure of aberrant MUC1 (adapted from FIG. 1 in Gao T. et al. Biomed Pharmacother. 2020). Cancer associated (CA) MUC1 epitopes include an exposed core peptide, MUC1-Tn, and MUC1-sTn.

FIG. 2—FACS data indicating specificity and relative affinity of MUC1 IgG's for MCF7 cells (graphs A; C; E; G; I; K; M; and O) compared to MCF10A cells (graphs B; D; F; H; J; L; N; and P). MUC1 IgG's include core binders (CB), Tn binders (TnB), and (S)Tn binders (SEQ ID NOs). Negative control antibody is SEQ ID NO: 216/217; positive control antibody is SEQ ID NO: 210/211 (A-P) and SEQ ID NO: 212/213 (O-P). X-axis: concentration of IgG in μg/ml; Y-axis: median PE signal x10⁴.

FIG. 3—Affinity for MUC1-Tn plotted against affinity for MUC1-STn of MUC1-(S)Tn binders.

X-axis: IgG affinity to MUC1-STn as measured in ELISA, expressed in AUC normalized to an analog of reference antibody 5E5 and isotype control. Y-axis: depicted Fab affinity to MUC1-Tn as measured in ELISA, expressed in AUC normalized to an analog of reference antibody 5E5 and isotype control. Squares in the circle are antibodies that represent two groups of antibodies based on a very similar HCDR1: 1) SEQ ID NO: 1; SEQ ID NO: 29; SEQ ID NO: 45; SEQ ID NO: 57; SEQ ID NO: 78; 2) SEQ ID NO: 5; SEQ ID NO: 9; SEQ ID NO:178.

FIG. 4—Glycopeptide array data providing EC50 values (ug/ml) of MUC1 IgG binding to MUC1-Tn glycopeptides T1-T12 and S1-S12. No values indicate that binding was undetectable.

FIG. 5—FACS data indicating affinity of MUC1 IgG's for T47D huGalNAc cells expressing MUC1-STn (graphs A-D) and T47D cells expressing MUC1-Tn (graphs E-H). Negative control antibody is SEQ ID NO: 216/217; positive control antibody is SEQ ID NO: 210/211 (A-H) and SEQ ID NO: 212/213 (D; H). X-axis: concentration of IgG in μg/ml; Y-axis: median fluorescence x10⁴.

FIG. 6—FACS data indicating affinity of MUC1 IgG's for MC38 huMUC1 COSMC KO cells (graphs A; C; D; F; G; I; and J) compared to MC38 huMUC1 cells (graphs B; E; H; and K). Negative control antibody is SEQ ID NO: 216/217; positive control antibody is SEQ ID NO: 210/211 (A-K) and SEQ ID NO: 212/213 (J-K). X-axis: concentration of IgG in μg/ml; Y-axis: median fluorescence×10⁴.

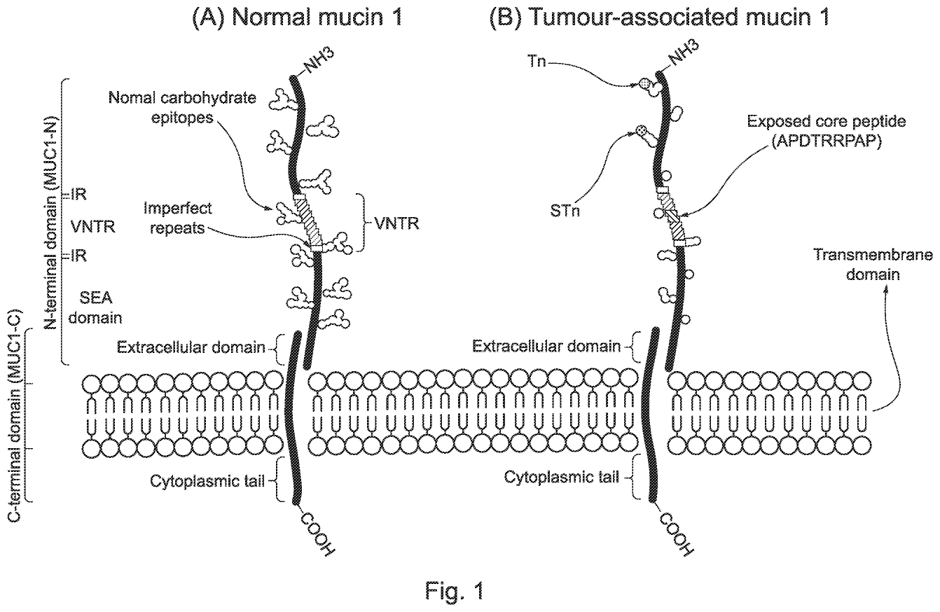

### EXAMPLES

In the Examples, which are used to illustrate the present disclosure but are not intended to limit the disclosure in any way, the MUC1 binding domains of the present disclosure are characterized in IgG format, wherein each MUC1 binding domain comprises a light chain variable region having an amino acid sequence as set forth in SEQ ID NO: 218 and a light chain constant region having an amino acid sequence as set forth in SEQ ID NO: 226, as well as in Fab format. In the Examples, which are used to illustrate the present disclosure but are not intended to limit the disclosure in any way, unless noted otherwise, MUC1 IgG were screened in IgG1 format, comprising a CH1 having an amino acid sequence as set forth in SEQ ID NO: 223, a CH2 having an amino acid sequence as set forth in SEQ ID NO: 224, and a CH3 having an amino acid sequence as set forth in SEQ ID NO: 225.

Reference antibodies and control antibodies used in the Examples include:
Reference antibody 5E5, which is an analog of bivalent monospecific humanized antibody 5E5 and comprises two heavy chains having an amino acid sequence as set forth in SEQ ID NO: 210 and two light chains having an amino acid sequence as set forth in SEQ ID NO: 211. Humanized 5E5 binds to the MUC1-(s)Tn (GS*T*A) epitope.
Reference antibody pankomab, which is an analog of bivalent monospecific antibody pankomab and comprises two heavy chains having an amino acid sequence as set forth in SEQ ID NO: 212 and two light chains having an amino acid sequence as set forth in SEQ ID NO: 213. Pankomab binds to the MUC1-(s)Tn (PDT*RP) epitope.
Reference antibody HT186-D11, which is an analog of bivalent monospecific antibody HT186-D11 and comprises two heavy chains having an amino acid sequence as set forth in SEQ ID NO: 214 and two light chain having an amino acid sequence as set forth in SEQ ID NO: 215. HT186-D11 binds to the MUC1 core (RPAP) epitope.
Bivalent monospecific control anti-STn antibody clone 3F1 (SBH Sciences, cat. no. SBH-ASTn), which binds to STn.
Bivalent monospecific anti-Tn antibody clone 5F4 (SBH Sciences, cat. no. SBH-Tn), which binds to Tn.
Bivalent monospecific control antibody VU4H5 (BioKE, cat. no. 4538S), which binds to the MUC1 core (APDTRPAP) epitope.
Bivalent monospecific isotype control IgG, comprising two heavy chains having an amino acid sequence as set forth in SEQ ID NO: 216 and two light chains having an amino acid sequence as set forth in SEQ ID NO: 217.

### Example 1—Generation of MUC1 Fabs

Binding domains, and antibodies, comprising heavy chain variable regions binding cancer-associated MUC-1 were obtained by immunizing transgenic mice comprising a common IGKV1-39 light chain (MeMo® mice). Several immunization methods were used, including immunization with KHL-conjugated human MUC1₄₀Tn₈ or MUC1₄₀Tn₁₀ peptides, plus cells overexpressing human MUC1-(S)Tn. Phage display libraries were generated and screened in ELISA and FACS using MUC1 peptides and cell lines to identify Fabs that bind MUC1-Tn and/or MUC1-STn (MUC1-(S)Tn). Binders were subsequently re-cloned into IgG format for further characterization. The binding domain sequences herein, once characterized and sequenced through the techniques provided herein, can be subsequently obtained by any method known in the art.

### Example 2—Specificity and Relative Affinity for MUC1-(S)Tn Measured Using ELISA

The panel of MUC1 IgG's was screened in an ELISA to characterize their binding specificity to, and relative affinity for, different MUC1 peptides. A mock peptide and a secondary antibody only were included as negative assay controls. An isotype control antibody and an analog of reference antibody 5E5 were included as negative and positive antibody controls, respectively.

Biotinylated MUC1 and control peptides were captured at concentrations indicated in Table 1 on a neutravidin (ThermoFisher Scientific, cat. no. 31000) coated Maxisorp plate, and incubated for one hour at room temperature. The MUC1 IgG and control antibodies were diluted in blocking buffer, added at 10, 1, 0.1 and 0.01 ug/ml, and incubated for one hour at room temperature. Subsequently, samples were incubated with diluted HRP-conjugated secondary antibodies (Bethyl Laboratories, A80-104P, 1: 2000) for one hour at room temperature. Three washing steps using PBST were performed after each incubation step. 1 M H₂SO₄ was used to stop the reaction and TMB peroxidase substrate solution (BD Biosciences, cat. no. 555214) was used to develop the signal. Plates were analyzed at A₄₅₀ using Microplate reader.

TABLE 1
Peptides used in ELISA.

| | | | Capture |
|---|---|---|---|
| MUC1 | | Modifi- | conc |
| peptide | Sequence and (s)Tn modification | cation | (μg/ml) |
| MUC140 | PAPGSTAPPAHGVTSAPDTRPAPGSTAPPAHGVTSAPDTR | None | 2 |
| MUC140- | PAPGS*T*APPAHGVT*S*APDT*RPAPGS*T*APPAHGVT*S*APDT*R | Tn | 2 |
| Tn10 | | | |
| MUC140- | PAPGS*T*APPAHGVT*S*APDT*RPAPGS*T*APPAHGVT*S*APDT*R | STn | 10 |
| STN10 | | | |
| Mock | Non MUC1 sequence | None | 2 |
| peptide | | | |

The assay identified many antibodies in the MUC1 IgG panel that bind MUC1₄₀Tn₁₀ and not to the mock peptide (data not shown). Binding specificity of the MUC1-(S)Tn antibodies was binned into three groups: 1) MUC1 core peptide binders (binding to MUC1₄₀ and MUC1₄₀Tn₁₀ and MUC1₄₀STn₁₀); 2) MUC1-Tn binders (binding to MUC1₄₀Tn₁₀ but not to MUC1₄₀STn₁₀); and 3) MUC1-(S)Tn binders (binding to MUC1₄₀Tn₁₀ and MUC1₄₀STn₁₀).

### Example 3—Specificity and Relative Affinity for MUC1-(S)Tn Measured Using FACS

The panel of MUC1 IgG's was screened on MCF7 cells that express cancer-associated MUC1-Tn and on MCF10A cells that express wildtype MUC1. An analog of reference antibody pankomab was included as a control of relative expression of MUC1 on the different cell lines. 293FF (MUC1 negative) cells were included as a negative assay control. An isotype control antibody and an analog of reference antibody 5E5 were included as negative and positive antibody controls, respectively. The MUC1 IgG panel and control antibodies were titrated using 8-step 4-fold serial dilution starting at 10 ug/ml. Binding to 293FF cells was tested at 10 ug/ml.

MCF7 Cells (DSMZ, ACC115), MCF10A cells (ATCC, cat. no. CRL-10317), or 293FF cells (Invitrogen, p/n51-0029) were incubated with MUC1 and control antibodies diluted in FACS buffer (PBS+0.5% BSA+2 mM EDTA) on ice for 30 minutes, followed by two washing steps with ice cold FACS buffer and staining with anti-human IgG-PE (Invitrogen, #H10104) at 1:100. Binding was measured using iQue Screener PLUS. Analysis was performed using IntelliCyt ForeCyt® Software. Mean fluorescence intensity (MFI) value of each MUC1 antibody was plotted against the concentration of the IgG's, and area under the curve (AUC) was calculated based on normalization using an isotype antibody and an analog of reference antibody 5E5 as negative and positive antibody controls, respectively.

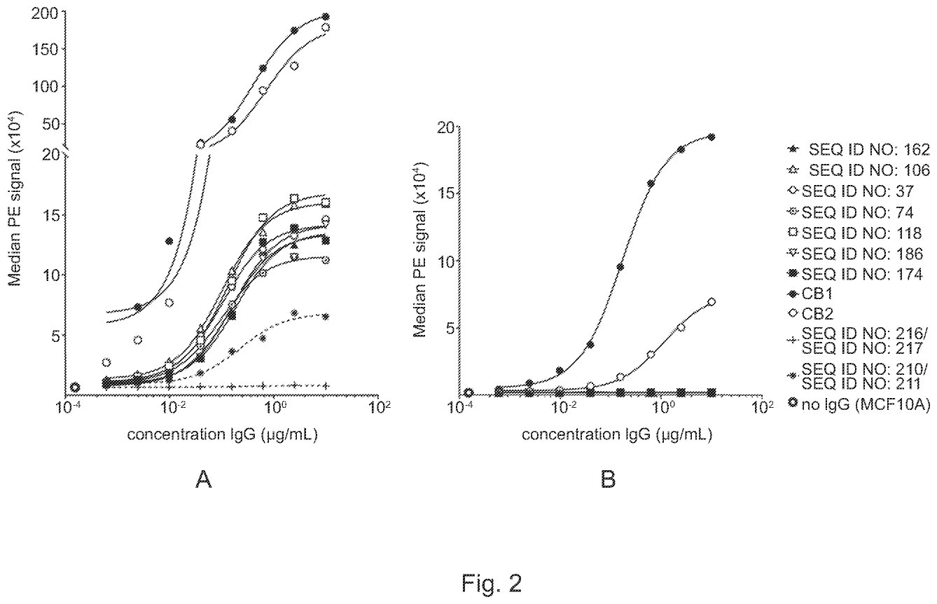

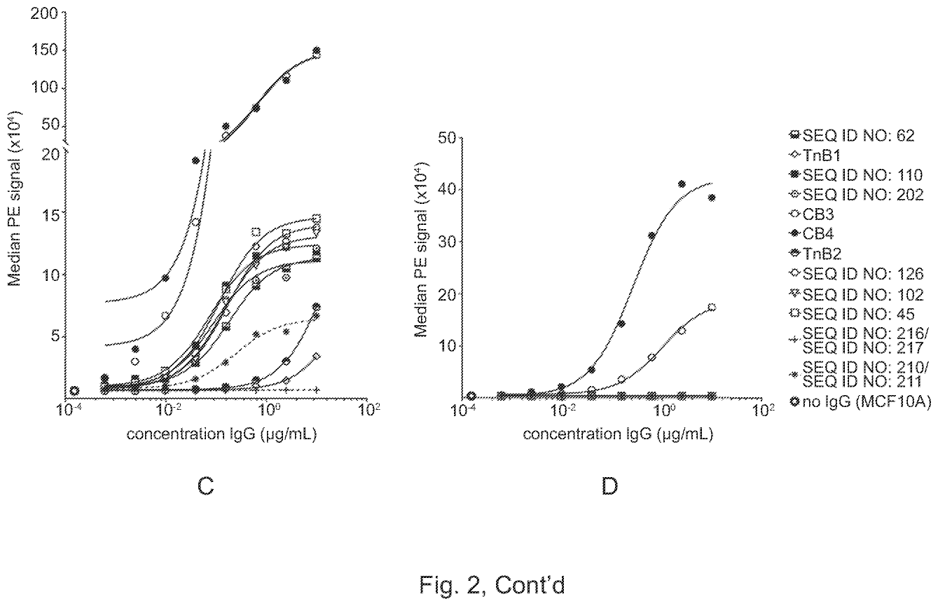

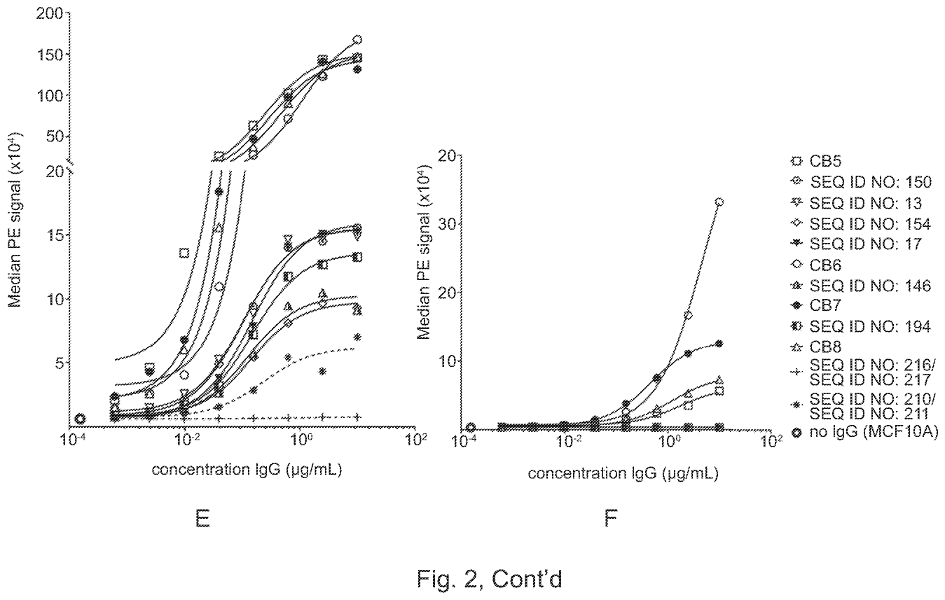

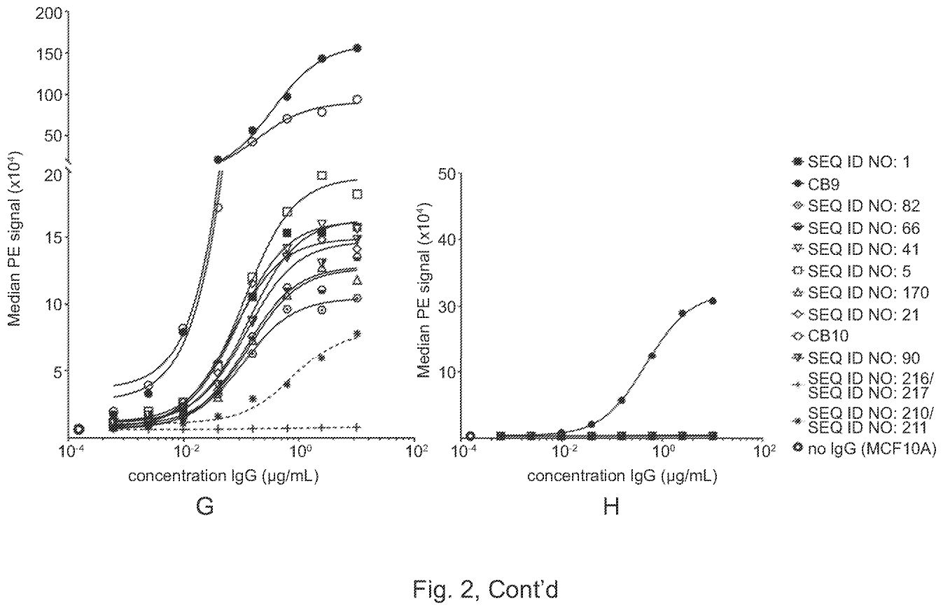

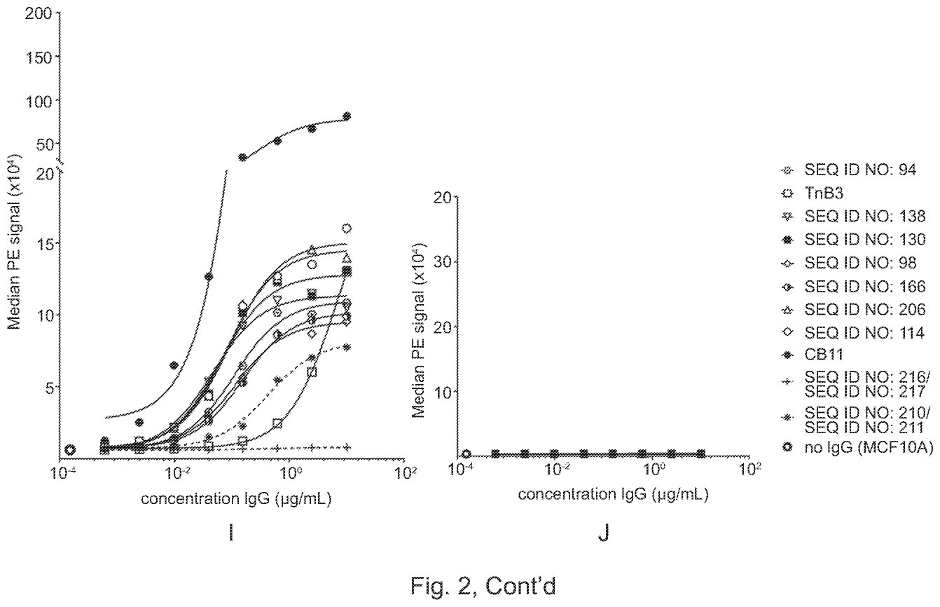

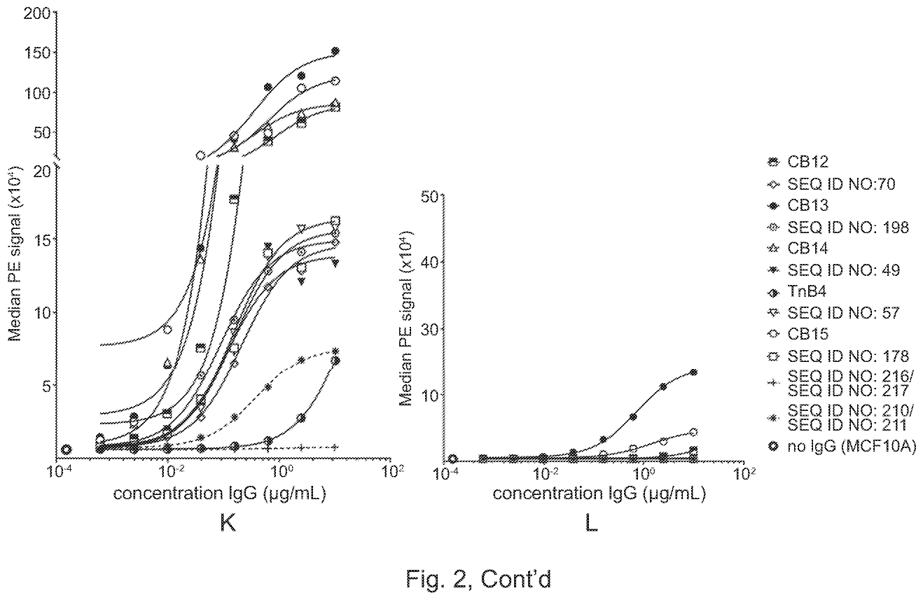

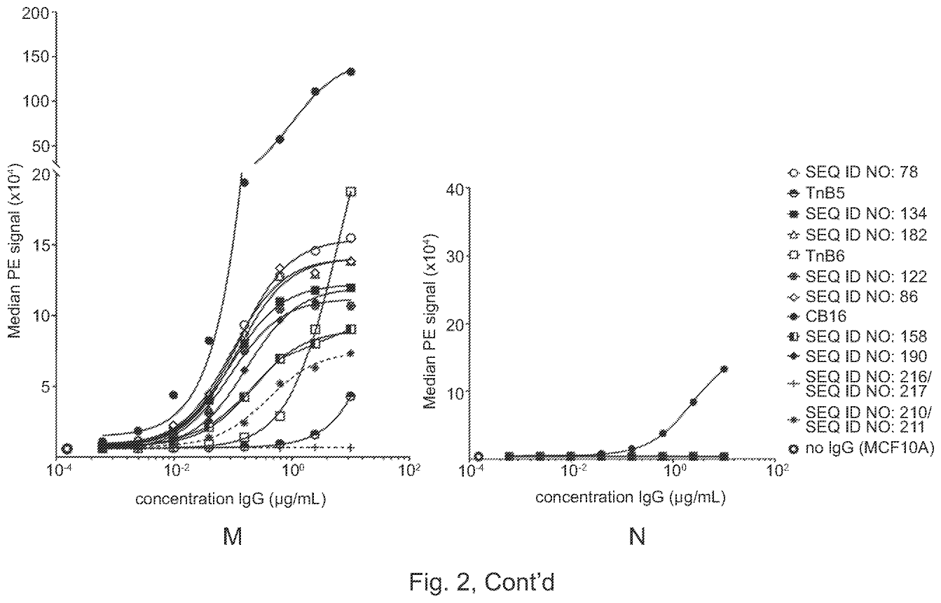

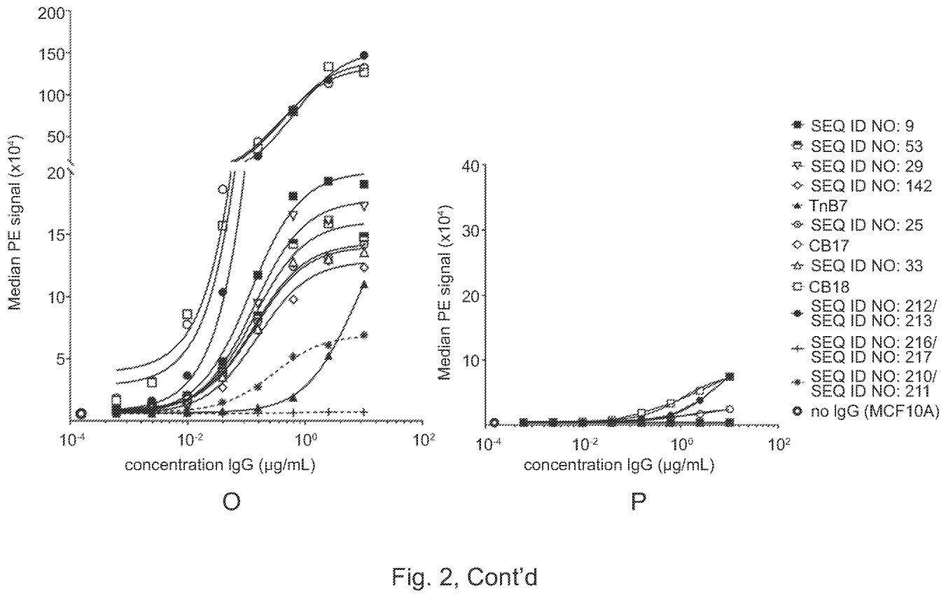

Results are shown in FIG. 2. Positive and negative control antibodies showed binding to MCF7, MCF10A, and 293FF cells as expected. MUC1 core peptide binders (CB) bind to both MCF7 and MCF10A cells, and a few showed low binding to 293FF cells. MUC1-(S)Tn binders (SEQ ID NOs) bind specifically to MCF7 but not to MCF10A cells. MUC1-Tn binders (TnB) bind specifically to MCF7 but not to MCF10A cells; however, with a lower affinity compared to MUC1-(S)Tn binders. The affinity of MUC1-(S)Tn binders to MCF7 cells is diverse with many antibodies showing a higher binding signal than the analog of reference antibody 5E5.

### Example 4—Affinity of MUC1 Fab Panel for MUC1₄₀Tn₁₀ and MUC1₄₀STn₁₀ Measured Using ELISA

The panel of MUC1-(S)Tn IgG's was subjected to GingisKHAN digestion to produce Fabs (>99% monomeric). Biotinylated MUC1₄₀Tn₁₀ and MUC1₄₀STn₁₀ were captured on a neutravidin coated plate at 2 μg/ml. Fab fragments of an analog of reference antibody 5E5 were included as a positive control antibody and Fab fragments of an isotype control antibody were included as a negative control antibody.

The panel of MUC1-(S)Tn Fabs and the control Fabs were serially diluted using 8-step 10-fold serial dilution starting at 10 ug/ml, and incubated for one hour at room temperature, followed by incubation with diluted HRP-conjugated secondary antibodies (Becton Dickinson, cat. no. 555788) for one hour at room temperature. Three washing steps using PBST were performed after each incubation step. 1 M H₂SO₄ was used to stop the reaction and TMB peroxidase substrate solution (BD Biosciences, cat. no. 555214) to develop the signal. Plates were analyzed at A₄₅₀ using Microplate reader.

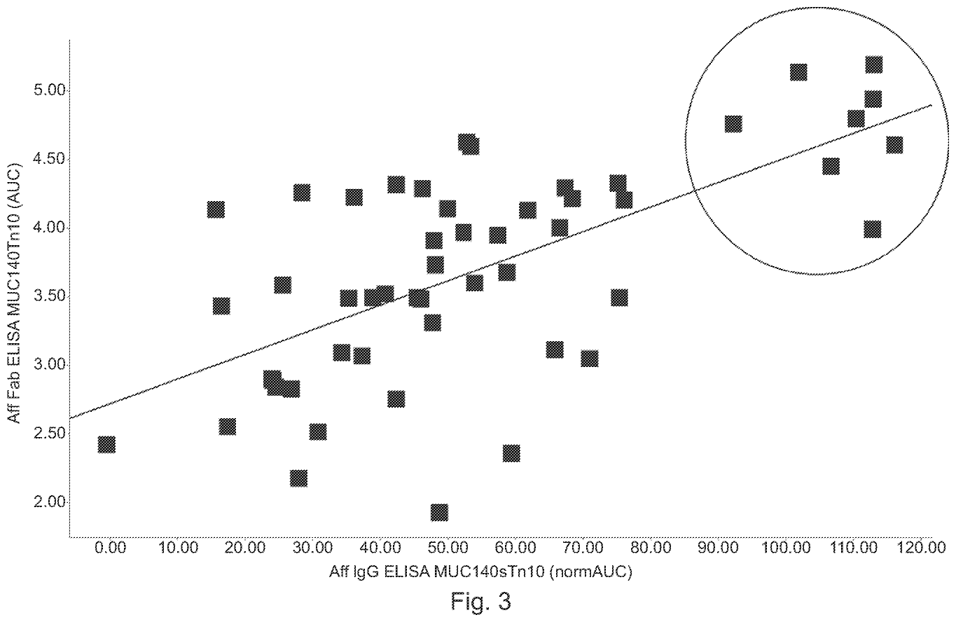

The affinity of the MUC1-(S)Tn Fabs to MUC1₄₀Tn₁₀ and MUC1₄₀STn₁₀ is similar to that of the IgG's (data not shown). A general correlation in affinity was found against MUC1-Tn peptide and MUC1-STn peptide (see FIG. 3). A number of clones show high affinity to MUC1-Tn and MUC1-STn. Within this group, many of the Fabs that show the highest binding affinity to MUC1-Tn and MUC1-STn share the same HCDR1 sequence (encircled in FIG. 3).

### Example 5—Glycopeptide Array

The panel of MUC1 IgG's was screened in a glycopeptide array to determine their binding to the O-glycopeptides listed in Table 2. As a negative assay control, print buffer was included. Positive assay controls were human IgG, and mouse IgG. Control antibodies included: an analog of reference antibody 5E5, an analog of reference antibody VU4H5, 5F4, and 3F1. MUC1 and control antibodies were tested at 8-step 4-fold serial dilution starting at 10 ug/ml.

The O-glycan array assay was performed according to the manual instructions (Z Biotech, LLC). The array was blocked for 30 minutes using Glycan Array Blocking Buffer (GAAB, Z Biotech Item #10106). It was then washed 3× using TBS-T-based Glycan Array Assay Buffer (GAAB, Z Biotech Item #10107). MUC1 IgG's and control antibodies were diluted in GAAB in a range from 10 μg/ml to 0.6 ng/ml, and then applied directly to the array. The array was covered with an adhesive film and shaken at 80 rpm for 1 hour at room temperature. The array was then washed 3× again with GAAB. The detection antibodies, Cy3-conjugated anti human IgG and Cy3-conjugated anti mouse IgG, were diluted in GAAB and applied to the array. It was covered from light and shaken at 80 rpm for 1 hour at room temperature, and then washed 3× with GAAB and 2× with MilliQ water. The array was read using an Innopsys InnoScan 710 Microarray Scanner with a high power laser at 1 PMT. Software was used to detect each spot on the array and calculate the relative fluorescence units (RFU) intensity for each spot. Background RFU was subtracted from each spot's RFU value. The median of each glycan's spots was determined and graphed on the report given.

TABLE 2
O-glycopeptides used in glycopeptide array.

| ID | Structures | ID |
|---|---|---|
| Tn-MUC1 | T1 | PAPGSTAPPAHGVTSAPDTRPAPG-OH |
| Tn-MUC1 | T2 | PAPGS(α-GalNAc)TAPPAHGVTSAPDTRPAPG-OH |
| Tn-MUC1 | T3 | PAPGST(α-GalNAc)APPAHGVTSAPDTRPAPG-OH |
| Tn-MUC1 | T4 | PAPGSTAPPAHGVT(α-GalNAc)SAPDTRPAPG-OH |
| Tn-MUC1 | T5 | PAPGSTAPPAHGVTS(α-GalNAc)APDTRPAPG-OH |
| Tn-MUC1 | T6 | PAPGSTAPPAHGVTSAPDT(α-GalNAc)RPAPG-OH |
| Tn-MUC1 | T7 | PAPGS(α-GalNAc)T(α-GalNAc)APPAHGVTSAPDTRPAPG-OH |
| Tn-MUC1 | T8 | PAPGST(α-GalNAc)APPAHGVTSAPDT(α-GalNAc)RPAPG-OH |
| Tn-MUC1 | T9 | PAPGSTAPPAHGVT(α-GalNAc)S(α-GalNAc)APDTRPAPG-OH |
| Tn-MUC1 | T10 | PAPGS(α-GalNAc)T(α-GalNAc)APPAHGVTSAPDT(a-GalNAc)RPAPG-OH |
| Tn-MUC1 | T11 | PAPGSTAPPAHGVT(α-GalNAc)SAPDT(α-GalNAc)RPAPG-OH |
| Tn-MUC1 | T12 | HYTNPSQDVTVPCPVTSTPPTPS(α-GalNAc)PSTPPTPSPSCCHPR-NH2 |
| sTn-MUC1 | S1 | PAPGSTAPPAHGVTSAPDTRPAPG-OH |
| sTn-MUC1 | S2 | PAPGS(Neu5Ac-α(2-6)-GalNAc)TAPPAHGVTSAPDTRPAPG-OH |
| sTn-MUC1 | S3 | PAPGST(Neu5Ac-α(2-6)-GalNAc)APPAHGVTSAPDTRPAPG-OH |
| sTn-MUC1 | S4 | PAPGSTAPPAHGVT(Neu5Ac-α(2-6)-GalNAc)SAPDTRPAPG-OH |
| sTn-MUC1 | S5 | PAPGSTAPPAHGVTS(Neu5Ac-α(2-6)-GalNAc)APDTRPAPG-OH |
| sTn-MUC1 | S6 | PAPGSTAPPAHGVTSAPDT(Neu5Ac-α(2-6)-GalNAc)RPAPG-OH |
| sTn-MUC1 | S7 | PAPGS(Neu5Ac-α(2-6)-GalNAc)T(Neu5Ac-a(2-6)-GalNAc) |
| | | APPAHGVTSAPDTRPAPG-OH |
| sTn-MUC1 | S8 | PAPGST(Neu5Ac-α(2-6)-GalNAc)APPAHGVTSAPDT(Neu5Ac-a(2-6)- |
| | | GalNAc)RPAPG-OH |
| sTn-MUC1 | S9 | PAPGSTAPPAHGVT(Neu5Ac-α(2-6)-GalNAc)S(Neu5Ac-a(2-6)- |
| | | GalNAc)APDTRPAPG-OH |
| sTn-MUC1 | S10 | PAPGS(Neu5Ac-α(2-6)-GalNAc)T(Neu5Ac-a(2-6)-GalNAc) |
| | | APPAHGVTSAPDT(Neu5Ac-α(2-6)-GalNAc)RPAPG-OH |
| sTn-MUC1 | S11 | PAPGSTAPPAHGVT(Neu5Ac-α(2-6)-GalNAc)SAPDT(Neu5Ac-a(2-6)- |
| | | GalNAc)RPAPG-OH |
| sTn-MUC1 | S12 | HYTNPSQDVTVPCPVTSTPPTPS(Neu5Ac-α(2-6)-GalNAc) |
| | | PSTPPTPSPSCCHPR-NH2 |
| MUC1 | O1 | GalNAcα-Ser |
| Fragments | | |
| MUC1 | O2 | GalNAcα-Thr |
| Fragments | | |
| MUC1 | O3 | Neu5Acα2-6GalNAcα-Ser |
| Fragments | | |
| MUC1 | O4 | Neu5Acα2-6GalNAcα-Thr |
| Fragments | | |
| MUC1 | O49 | H2N-APGST(α-GalNAc)APP-NH2 |
| Fragments | | |
| MUC1 | O50 | H2N-TSAPDT(α-GalNAc)RPAP-NH2 |
| Fragments | | |

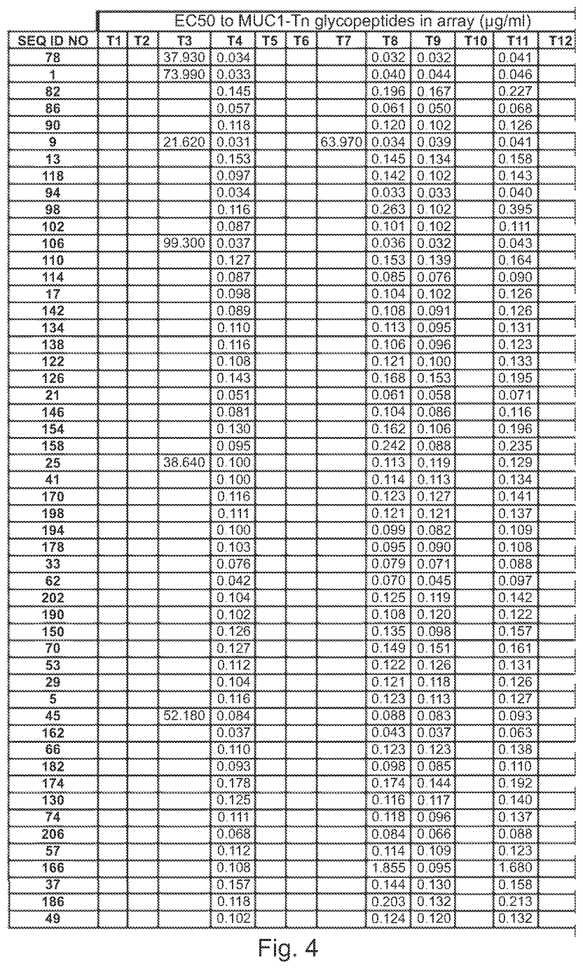

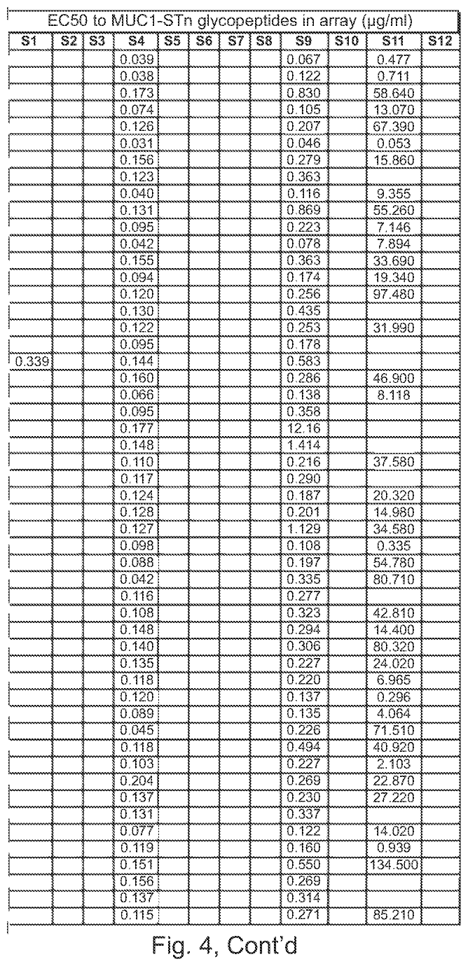

All control antibodies bound to their described epitopes. Results of the MUC1 IgG's are shown in FIG. 4. MUC1 IgG previously identified as MUC1-(S)Tn binders showed strong binding to peptides T4, T9, T11, S4, S9, and S11. These peptides all comprise a modification of the threonine residue in the VTSA motif, either a Tn modification (α-GalNAc glycosylation) or a STn modification (Neu5Ac-α(2-6)-GalNAc glycosylation). None of the other peptides comprise a modification at this position and the MUC1-(S)Tn binders do not bind to those peptides, apart from peptide T8. The MUC1-(S)Tn binders also bind the peptides when, in addition to the (S)Tn modification of the threonine residue in the VTSA motif, the peptide comprises a Tn or STn modification of the serine residue in the VTSA motif (T9, S9) or a Tn or STn modification of the threonine residue in the PDTR motif (T11, S11).

MUC1 IgG previously identified as MUC1-Tn binders showed strong binding to peptides T2, T7, and T10. These peptides all comprise a Tn modification (α-GalNAc glycosylation) of the serine residue in the GSTA motif. None of the other peptides comprise a modification at this position and the MUC1-(S)Tn binders do not bind to those peptides. The MUC1-Tn binders also bind the peptides when, in addition to the Tn modification of the serine residue in the GSTA motif, the peptide comprises a Tn modification of the threonine residue in the GSTA motif (T7) or a Tn modification of the threonine residue in the GSTA motif and a Tn modification of the threonine residue in the PDTR motif (T10).

### Example 6—Affinity of MUC1 IgG for T47D huGalNAc Cells

MUC1 IgG's were screened for binding to T47D huGalNAc cells stably expressing human MUC1-STn, and T47D WT cells expressing medium levels of MUC-Tn. An isotype control antibody was included as a negative control antibody, and an analog of reference antibody 5E5 was included as a positive control antibody. An analog of reference antibody pankomab was included as a control of relative expression of MUC1 on the different cell lines. Further control antibodies included 3F1 and 5F4. MUC1, the isotype control, analog of 5E5 and analog of pankomab antibodies were titrated using 8-step 4-fold serial dilution starting at 10 ug/ml; 3F1 and 5F4 were used at 10 ug/ml.

T47D cells (Sigma-Aldrich, cat. 85102201-1VL) and T47D huGalNAc cells (generated by stably transfecting T47D cells with plasmid encoding ST6GALNAC1 (accession no. Q9NSC7; Uniprot)) were incubated with MUC1 IgG's and control antibodies diluted in FACS buffer (PBS+0.5% BSA+2 mM EDTA) for 30 minutes on ice, followed by two washing steps with ice cold FACS buffer and staining with anti-human IgG-PE (Invitrogen, cat. no. H10104) at 1:100 (for the MUC1, the isotype control, analog of 5E5, and analog of pankomab antibody samples) or anti-mouse IgG-PE (Invitrogen, cat. no. M30004-4) at 1:400 (for the 3F1 and 5F4 antibody samples). Binding was measured using iQue Screener PLUS. Analysis was performed using IntelliCyt ForeCyt® Software. MFI value of each MUC1 antibody was plotted against the concentration of IgG's, and area under the curve (AUC) was calculated based on normalization using an isotype control antibody and an analog of reference antibody 5E5 as negative and positive antibody controls, respectively.

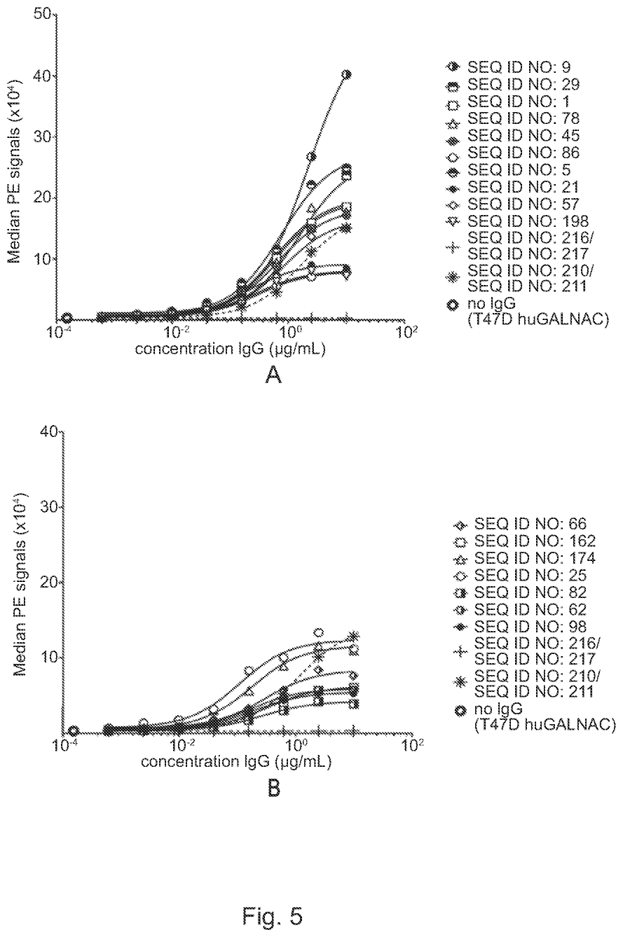

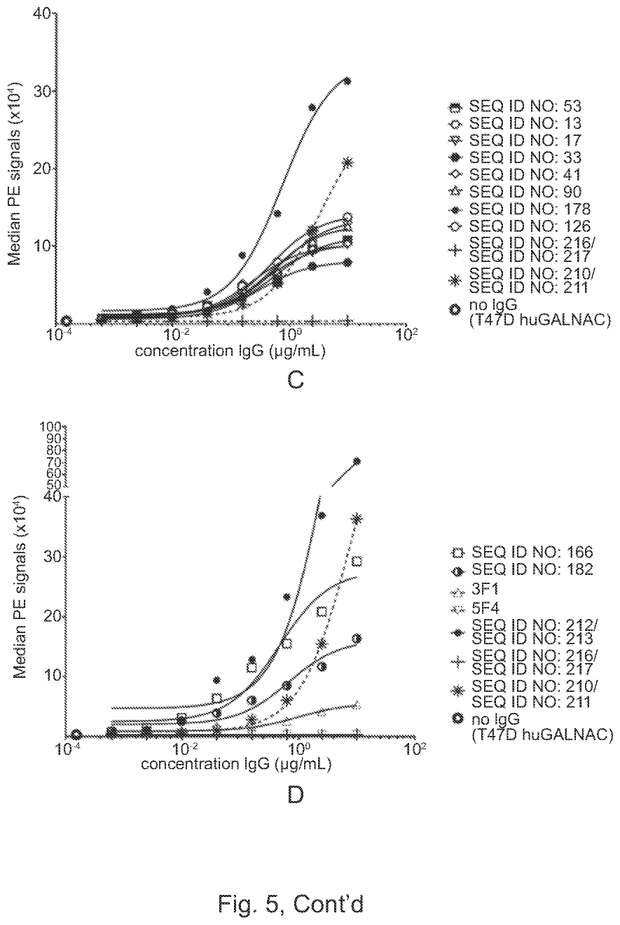

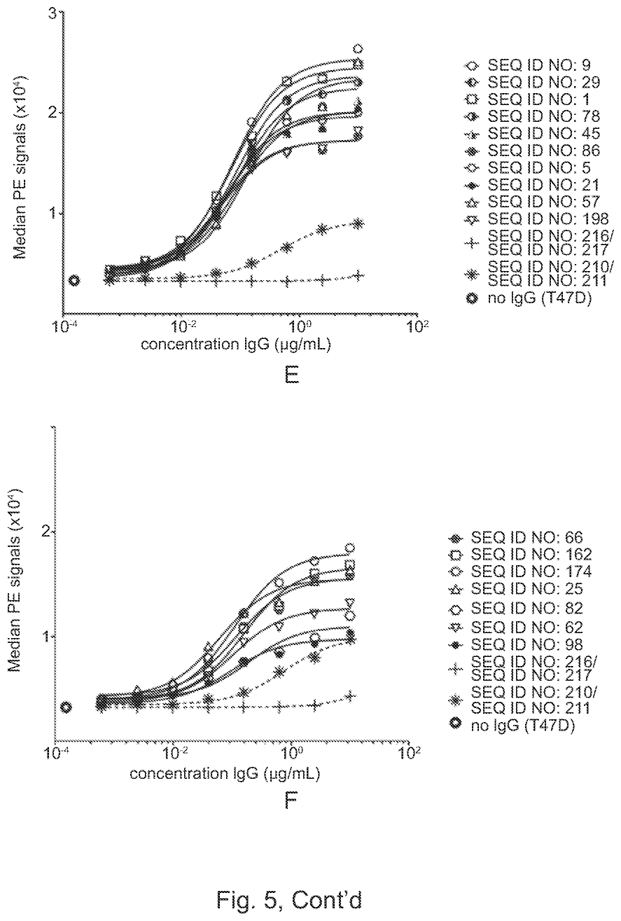

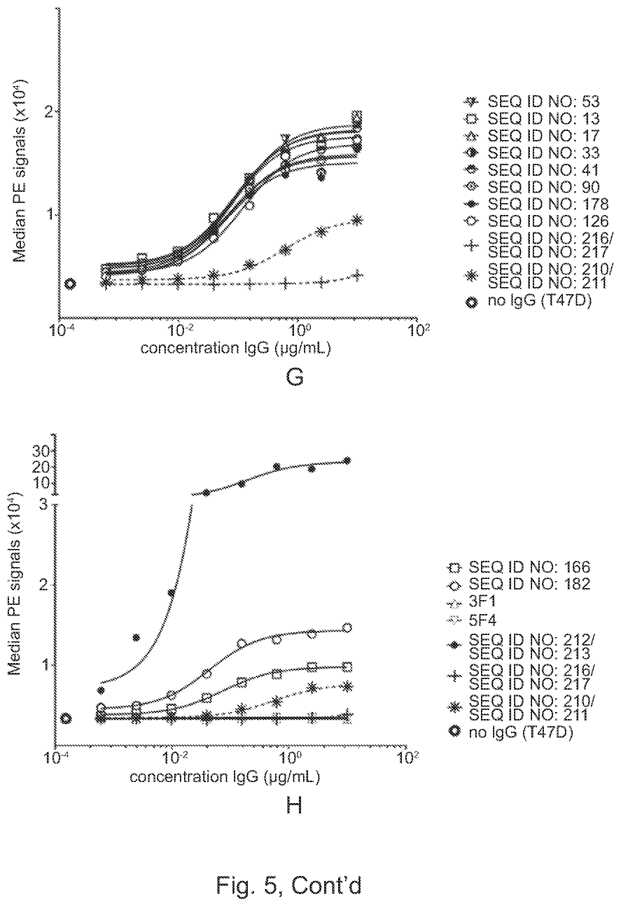

Results are shown in FIG. 5. The panel of MUC1 (S)Tn binders shows diverse binding affinity to T47D huGalNAc cells. A number of antibodies show higher maximum binding than the analog of reference antibody 5E5. The affinity to T47D huGalNAc cells correlate well with affinity to MUC1-STn peptide.

The panel of MUC1-(S)Tn binders show diverse binding affinity to T47D WT cells. Most of the antibodies show a higher maximum binding than the analog of reference antibody 5E5, corresponding to the data obtained with MCF7 cells. The affinity to T47D WT cells further correlate well with the affinity to MUC1₄₀Tn₁₀ peptide.

### Example 7—Affinity of MUC1 IgG for MC38 Cells

MUC1 IgG's were screened for binding to MC38 huMUC1 COSMC KO cells that express human MUC1-Tn, MC38 huMUC1 cells that express normal human MUC1 glycoform, and MC38 WT cells that do not express human MUC1. An isotype control antibody was included as a negative control antibody, and an analog of reference antibody 5E5 was included as a positive control antibody. An analog of reference antibody pankomab was included as a control of relative expression of MUC1 on the different cell lines. Further control antibodies included 3F1 and 5F4. The MUC1 IgG's, the isotype control, an analog of 5E5 and an analog of pankomab were titrated using 8-step 4-fold serial dilution starting at 10 ug/ml; 3F1 and 5F4 were used at 10 ug/ml.

MC38 huMUC1 COSMC KO cells were generated by stable transfection of MC38 huMUC1 cells (accession no. CVCL_5138; Uniprot) with human MUC1 and knocking out the mouse COSMC (C1galt1c1) gene (accession no. ENSMUST00000058265.7; Ensemble)). Cells were incubated with MUC1 IgG's or control antibodies diluted in FACS buffer (PBS+0.5% BSA+2 mM EDTA) for 30 minutes on ice, followed by two washing steps with ice cold FACS buffer and staining with secondary antibodies anti human IgG-PE (Invitrogen, cat. no. H10104) at 1:100 (for the MUC1, the isotype control, analog of 5E5, and analog of pankomab antibody samples) or anti-mouse IgG-PE (Invitrogen, cat. no. M30004-4) at 1:400 (for the 3F1 and 5F4 antibody samples). Binding was measured using iQue Screener PLUS. Analysis was performed using IntelliCyt ForeCyt® Software. MFI value of each huMUC1-(S)Tn clone was plotted against the concentration of IgG's, and area under the curve (AUC) was calculated based on normalization using an isotype control antibody and an analog of reference antibody 5E5 as negative and positive antibody controls, respectively.

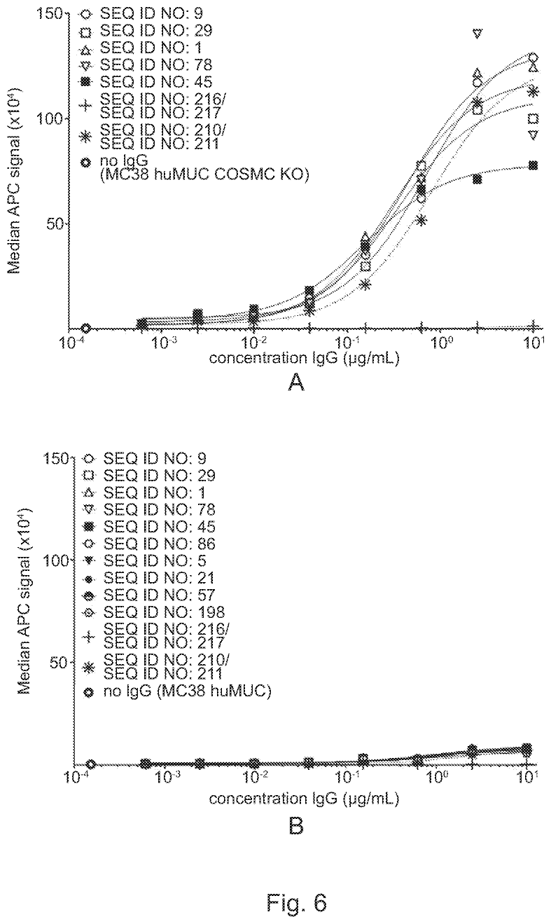

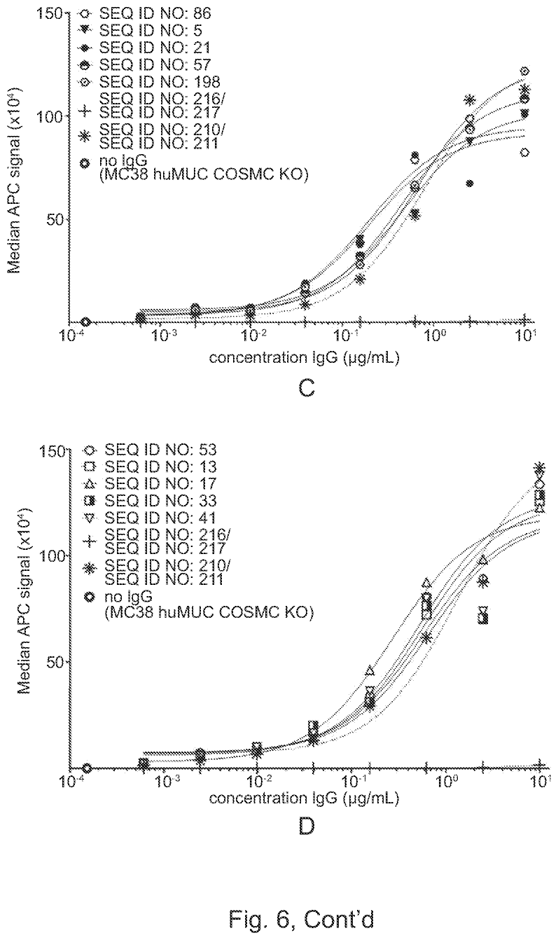

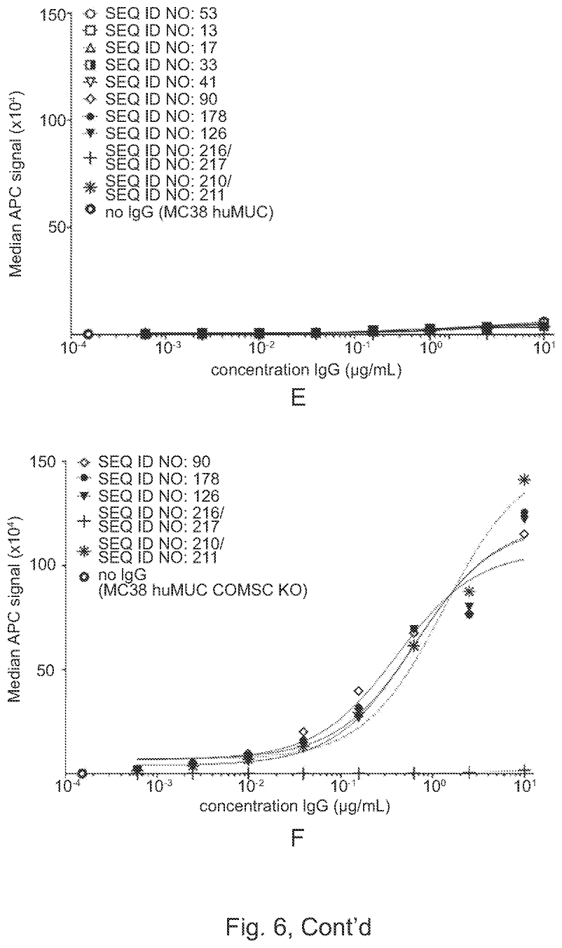

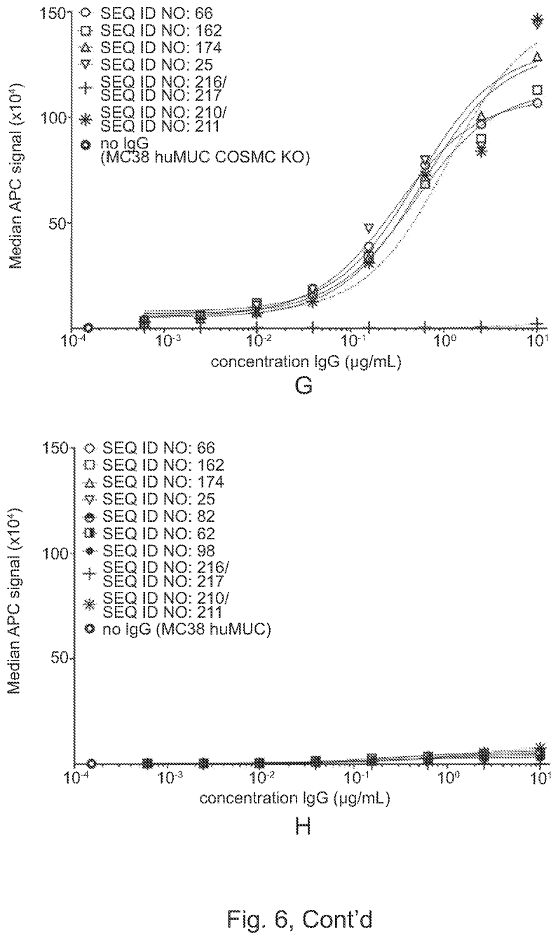

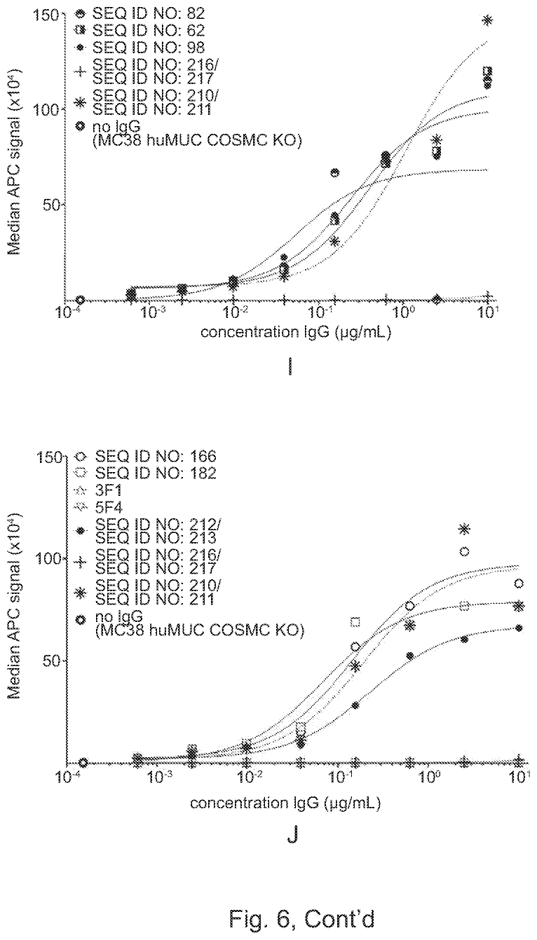

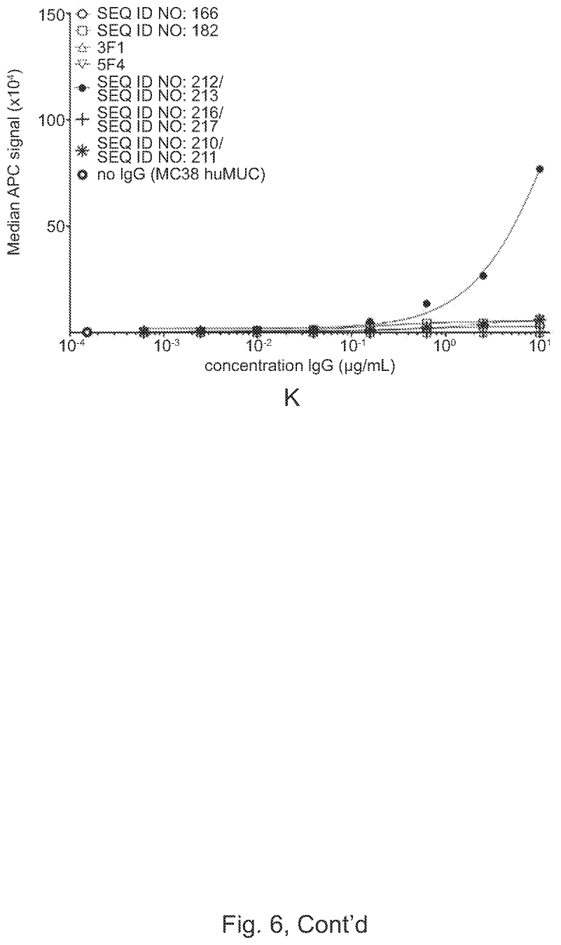

Results are shown in FIG. 6. Positive and negative control antibodies behave as expected on MC38 huMUC1 COSMC KO cells, MC38 huMUC1 cells, and MC38 WT cells. None of the MUC1 antibodies bind to MC38 WT cells, except for the antibody comprising a heavy chain variable region having SEQ ID NO: 178. This antibody, however, did not bind to CALR WT negative control peptide or 293FF WT cells. The panel of MUC1-(S)Tn binders show a high binding signal to MC38 huMUC1 COSMC KO cells. The panel of MUC1-(S)Tn binders show a very low binding signal to MC38 huMUC1 cells, indicating specificity for MUC1-(S)Tn.

SEQUENCES

SEQ ID NO: 1-Heavy chain variable region
QVQLVESGAEVKKPGASVRLSCKASGYSFIHYYLHYVRQAPGQGLEWMGVINPFDGSTDYAQKFHGRVT
LTRDTSASTVFMELSSLGSEDTAVYFCTRDNMIGYVLFDYWGLGTLVTVSS
SEQ ID NO: 2-Heavy chain CDR1
HYYLH
SEQ ID NO: 3-Heavy chain CDR2
VINPFDGSTDYAQKFHG
SEQ ID NO: 4-Heavy chain CDR3
DNMIGYVLFDY
SEQ ID NO: 5-Heavy chain variable region
QVQLVQSGAEVKKPGASVKVSCKTSGYTFTRYYIQWVRQAPGQGLEWMGVINPYDGSTNYAQKFQGRVT
VTRDTSTFTVYMELSSLRSEDTAVYYCARDYMRGYVIFDYWGQGTLVTVSS
SEQ ID NO: 6-Heavy chain CDR1
RYYIQ
SEQ ID NO: 7-Heavy chain CDR2
VINPYDGSTNYAQKFQG
SEQ ID NO: 8-Heavy chain CDR3
DYMRGYVIFDY
SEQ ID NO: 9-Heavy chain variable region
QVQLVQSGAEVKKPGASVKVSCKASGYTFNRHYIQWVRQAPGQGLEWMGTINPYDGSTHYAQKFQGRV
TLTRDTSTTTVYMELSSLRSEDTAVYYCARDYMIGYVVFDYWGQGTLVTVSS
SEQ ID NO: 10-Heavy chain CDR1
RHYIQ
SEQ ID NO: 11-Heavy chain CDR2
TINPYDGSTHYAQKFQG
SEQ ID NO: 12-Heavy chain CDR3
DYMIGYVVFDY
SEQ ID NO: 13-Heavy chain variable region
QVQLVQSGAEVKKPGASLKVSCKASGYTFTNYYIQWVRQAPGQGLEWMGVINPYDGSTNYAQKFQGRV
TLTRDTSTTTVYMELSSLRSDDTAVYYCARDYMIGYVVFDYWGQGTLVTVSS
SEQ ID NO: 14-Heavy chain CDR1
NYYIQ
SEQ ID NO: 15-Heavy chain CDR2
VINPYDGSTNYAQKFQG
SEQ ID NO: 16-Heavy chain CDR3
DYMIGYVVFDY
SEQ ID NO: 17-Heavy chain variable region
QVQLVQSGAEVKKPGASVRVSCKASGYPFINYYIQWVRQAPGQGLEWMGVVNPYDGSTNYAQKFQGRV
SITRDASTSTVFMELNSLRSEDTAVYYCARDSMLGHVIFDFWGQGTLVTVSS
SEQ ID NO: 18-Heavy chain CDR1
NYYIQ
SEQ ID NO: 19-Heavy chain CDR2
VVNPYDGSTNYAQKFQG
SEQ ID NO: 20-Heavy chain CDR3
DSMLGHVIFDF
SEQ ID NO: 21-Heavy chain variable region
QVQLVQSGAEVRKPGASVKFSCKASGYSFTHYYVHWVRQAPGQGLEWMGVINPFDSSTNYARKFQGRVT
MTRDTSTTTVYMDLSSLRSEDTAVYFCARDAMEGDSYFDYWGQGTLVTVSS
SEQ ID NO: 22-Heavy chain CDR1
HYYVH
SEQ ID NO: 23-Heavy chain CDR2
VINPFDSSTNYARKFQG
SEQ ID NO: 24-Heavy chain CDR3
DAMEGDSYFDY
SEQ ID NO: 25-Heavy chain variable region
QVQLVESGAEVKKPGASVKVSCKASGYTFARYYVHWVRQAPGQGLEWMGLINPYGGATNYAQNFQGRV
TMTRDTSTSTVYMELSSLRSEDTAVYYCARDAMGGDSYFDYWGQGTLVTVSS
SEQ ID NO: 26-Heavy chain CDR1
RYYVH
SEQ ID NO: 27-Heavy chain CDR2
LINPYGGATNYAQNFQG
SEQ ID NO: 28-Heavy chain CDR3
DAMGGDSYFDY
SEQ ID NO: 29-Heavy chain variable region
QVQLVQSGAEVKKPGASVKVSCKASGYSFIHYYIHWVRQAPGQGLEWMGVINPFDGSTNYAQKFQDRVT
VTRDTSTSTVFMDLSSLRSEDTAVYYCARDNMIGYVLFDDWGQGTLVTVSS
SEQ ID NO: 30-Heavy chain CDR1
HYYIH
SEQ ID NO: 31-Heavy chain CDR2
VINPFDGSTNYAQKFQD
SEQ ID NO: 32-Heavy chain CDR3
DNMIGYVLFDD
SEQ ID NO: 33-Heavy chain variable region
QVQLVQSGAEVKKPGASVRVSCKASGYPFINYYIQWVRQAPGQGLEWMGVINPFDGSTNYAQKFQDRVT
VTRDTSTSTVFMDLSSLRSEDTAVYYCARDNMIGYVLLDYWGQGTLVTVSS
SEQ ID NO: 34-Heavy chain CDR1
NYYIQ
SEQ ID NO: 35-Heavy chain CDR2
VINPFDGSTNYAQKFQD
SEQ ID NO: 36-Heavy chain CDR3
DNMIGYVLLDY
SEQ ID NO: 37-Heavy chain variable region
QVQLVQSGAEVKKPGASVTVSCKTSGYTFTSYYIHWLRQAPGQGLEWMGVINPFGGATNYAQKFQGRVT
MTRDTSTITVYMELSSLRSEDTAVYYCARDAMGGDSHFDHWGRGTLVTVSS
SEQ ID NO: 38-Heavy chain CDR1
SYYIH
SEQ ID NO: 39-Heavy chain CDR2
VINPFGGATNYAQKFQG
SEQ ID NO: 40-Heavy chain CDR3
DAMGGDSHFDH
SEQ ID NO: 41-Heavy chain variable region
QVQLVQSGAEVKEPGASMTLSCKTSGYTFISNYIHWLRQAPGQGLEWLGVINPFGGATNYAQKFQGRVTM
TRDTSTITVYMELSSLRSEDTAVYYCARDAMGGDSHFDYWGQGTLVTVSS
SEQ ID NO: 42-Heavy chain CDR1
SNYIH
SEQ ID NO: 43- Heavy chain CDR2
VINPFGGATNYAQKFQG
SEQ ID NO: 44-Heavy chain CDR3
DAMGGDSHFDY
SEQ ID NO: 45-Heavy chain variable region
QVQLVESGTEVKKPGASVKVSCKASGYSFIHYYIHWVRQAPGRGLEWMGVINPFDGSTNSAQNFQGRVTV
TRDTSTSTVFMDLSSLRSEDTAVYYCARDNMLGYVLFDHWGQGTLVTVSS
SEQ ID NO: 46-Heavy chain CDR1
HYYIH
SEQ ID NO: 47-Heavy chain CDR2
VINPFDGSTNSAQNFQG
SEQ ID NO: 48-Heavy chain CDR3
DNMLGYVLFDH
SEQ ID NO: 49-Heavy chain variable region
QVQLVESGAEVKKPGASVKVSCKASGYTFTRYYLHWVRQAPGQGLEWMGVINPYGGATNYAQKFQGRV
TLTRDTSTSTVYMELNSLRSEDTAVYFCARDAMEGNSYFDHWGPGTLVTVSS
SEQ ID NO: 50-Heavy chain CDR1
RYYLH
SEQ ID NO: 51-Heavy chain CDR2
VINPYGGATNYAQKFQG
SEQ ID NO: 52-Heavy chain CDR3
DAMEGNSYFDH
SEQ ID NO: 53-Heavy chain variable region
QVQLVQSGAEVKKPGASMTVSCKTSGYTFISYYIHWLRQAPGQGLEWLGVINPFGGATNYAQKFQGRVT
MTRDTSTITVYMELSSLRSEDTAVYYCARDAMGGDSHFDFWGQGTLVTVSS
SEQ ID NO: 54-Heavy chain CDR1
SYYIH
SEQ ID NO: 55-Heavy chain CDR2
VINPFGGATNYAQKFQG
SEQ ID NO: 56-Heavy chain CDR3
DAMGGDSHFDF
SEQ ID NO: 57-Heavy chain variable region
QVQLVESGTEVKKPGASVKVSCKASGYSFIHYYIHWVRQAPGRGLEWMGVINPFDGSTNYAQNFQGRVT
VTRDTSTSTVFMDLSSLRSEDTAVYYCARDNMIGYVLFDHWGQGTLVTVSS
SEQ ID NO: 58-Heavy chain CDR1
HYYIH
SEQ ID NO: 60-Heavy chain CDR2
VINPFDGSTNYAQNFQG
SEQ ID NO: 61-Heavy chain CDR3
DNMIGYVLFDH
SEQ ID NO: 62-Heavy chain variable region
QVQLVESGAEVKKPGASVKVSCKASGYSFTSYYIQWVRQAPGQGLEWLAVINPIDGSTNYAQKFQGRVTV
TRDTSTSTVYMELSSLRSEDTAVYYCARDYMRGFVVFDYWGQGTLVTVSS
SEQ ID NO: 63-Heavy chain CDR1
SYYIQ
SEQ ID NO: 64-Heavy chain CDR2
VINPIDGSTNYAQKFQG
SEQ ID NO: 65-Heavy chain CDR3
DYMRGFVVFDY
SEQ ID NO: 66-Heavy chain variable region
QVQLVQSGAEVKKPGASVRVSCKASGYTFTNYYIHWVRQAPGHGLTWMGVINPFDSSTDYAQKFQGRVT
MTRDTSTSTVYMDLSSLRSEDTAVYYCARDYMRGHVVFDYWGQGTLVTVSS
SEQ ID NO: 67-Heavy chain CDR1
NYYIH
SEQ ID NO: 68-Heavy chain CDR2
VINPFDSSTDYAQKFQG
SEQ ID NO: 69-Heavy chain CDR3
DYMRGHVVFDY
SEQ ID NO: 70-Heavy chain variable region
QVQLVQSGAEVKKPGASVKVSCKASGYAFPNYYIHWVRQAPGQGLEWMGVINPYGGSTNYAQKFQGRV
TMTRGTSTSTVYLELSSLRSEDTAVYYCARDAMKGDSFFDYWGQGTLVTVSS
SEQ ID NO: 71-Heavy chain CDR1
NYYIH
SEQ ID NO: 72-Heavy chain CDR2
VINPYGGSTNYAQKFQG
SEQ ID NO: 73-Heavy chain CDR3
DAMKGDSFFDY
SEQ ID NO: 74-Heavy chain variable region
QVQLVQSGAEVKKPGASVQVSCKASGYTFINYYLQWVRQAPGQGLEWMGTINPFDGSTHYEQNFQGRVT
MTRDTSTNTVYMELNGLRSEDTAVYYCARDHMIGYILFDYWGQGTTVTVSS
SEQ ID NO: 75-Heavy chain CDR1
NYYLQ
SEQ ID NO: 76-Heavy chain CDR2
TINPFDGSTHYEQNFQG
SEQ ID NO: 77-Heavy chain CDR3
DHMIGYILFDY
SEQ ID NO: 78-Heavy chain variable region
QVQLVQSGAEVKKPGTSVKVSCKASGYSFIHYYIHWVRQAPGQGLEWVGVINPFDGSTNYAQKFQGRVT
VTRDTSTSAVFMDLSSLRSEDTAVYYCARDNMIGYVLFDYWGQGTLVTVSS
SEQ ID NO: 79-Heavy chain CDR1
HYYIH
SEQ ID NO: 80-Heavy chain CDR2
VINPFDGSTNYAQKFQG
SEQ ID NO: 81-Heavy chain CDR3
DNMIGYVLFDY
SEQ ID NO: 82-Heavy chain variable region
QVQLVQSGAEVKKPGASVKVSCKASGYTFTSYYIHWVRQAPGQGLEWMGVINPYDSSTNYAQKFQGRVT
MTRDTSTSTVFMDLSSLRSEDTAVYYCARDNMIGYVLFDYWGQGTLVTVSS
SEQ ID NO: 83-Heavy chain CDR1
SYYIH
SEQ ID NO: 84-Heavy chain CDR2
VINPYDSSTNYAQKFQG
SEQ ID NO: 85-Heavy chain CDR3
DNMIGYVLFDY
SEQ ID NO: 86-Heavy chain variable region
QVQLVQSGAEVKKPGASVKVSCKASGYPFINYYIQWVRQAPGQGLEWMGVINPFDGSTNYAQKFQGRVT
VTRDTSTSTVFMDLSSLRSEDTAVYYCARDNMIGYVLFDYWGQGTLVTVSS
SEQ ID NO: 87-Heavy chain CDR1
NYYIQ
SEQ ID NO: 88-Heavy chain CDR2
VINPFDGSTNYAQKFQG
SEQ ID NO: 89-Heavy chain CDR3
DNMIGYVLFDY
SEQ ID NO: 90-Heavy chain variable region
QVQLVQSGAEVKKPGASVKVSCKASGYTFINHYIHWVRQAPGQGLEWMGVINPFDGSTNYAQKFQGRVT
VTRDTSTSTVFLDLSSLRSEDTAVYYCARDNMIGYVLFDYWGQGTLVTVSS
SEQ ID NO: 91-Heavy chain CDR1
NHYIH
SEQ ID NO: 92-Heavy chain CDR2
VINPFDGSTNYAQKFQG
SEQ ID NO: 93-Heavy chain CDR3
DNMIGYVLFDY
SEQ ID NO: 94-Heavy chain variable region
QVQLVQSGAEVKKPGSSVKVSCKASGYTFNSYYIHWVRQAPGQGLTWMGVINPFDGSTDYAQKFQGRVT
MTRDTSTSTVYMDLSSLRSEDTAVYYCARDYMRGYVVFDYWGRGTLVTVSS
SEQ ID NO: 95-Heavy chain CDR1
SYYIH
SEQ ID NO: 96-Heavy chain CDR2
VINPFDGSTDYAQKFQG
SEQ ID NO: 97-Heavy chain CDR3
DYMRGYVVFDY
SEQ ID NO: 98-Heavy chain variable region
QVQLVQSGAEVKKPGASVKVSCKASGYTFTSYYIQWLRQAPGQGLEWMGVINPTGGATNYAQKFQGRVT
MTRETSTSMVHMELSSLRSEDTAVYYCARDYMRGYVVFDYWGQGTLVTVSS
SEQ ID NO: 99-Heavy chain CDR1
SYYIQ
SEQ ID NO: 100-Heavy chain CDR2
VINPTGGATNYAQKFQG
SEQ ID NO: 101-Heavy chain CDR3
DYMRGYVVFDY
SEQ ID NO: 102-Heavy chain variable region
QVQLVQSGAEVKKSGASVKVSCKASGYPFISYYIHWLRQAPGQGLTWMGVINPFDGSTDYAQKFQGRVT
SEQ ID NO: 103-Heavy chain CDR1
SYYIH
SEQ ID NO: 104-Heavy chain CDR2
VINPFDGSTDYAQKFQG
SEQ ID NO: 105-Heavy chain CDR3
DYMRGYVVFDY
SEQ ID NO: 106-Heavy chain variable region
QVQLVQSGAEMKKPGASVKISCKASGYTFTNYYIQWVRQAPGQGLEWMGVINPYDGSTNYAQRFQGRVT
LTRDTSTTTVYMELSSLRSEDTAVYYCARDYMIGYVVFDFWGQGTLVTVSS
SEQ ID NO: 107-Heavy chain CDR1
NYYIQ
SEQ ID NO: 108-Heavy chain CDR2
VINPYDGSTNYAQRFQG
SEQ ID NO: 109-Heavy chain CDR3
DYMIGYVVFDF
SEQ ID NO: 110-Heavy chain variable region
QVQLVQSGAEMKKPGASVKVSCKASGYTFTNYYIHWVRQAPGQGLEWMGVINPYDGSTNYAQKFQGRV
TLTRDTSTTTVYMELSSLRSEDTAVYYCARDYMIGYVVFDFWGQGTLVTVSS
SEQ ID NO: 111-Heavy chain CDR1
NYYIH
SEQ ID NO: 112-Heavy chain CDR2
VINPYDGSTNYAQKFQG
SEQ ID NO: 113-Heavy chain CDR3
DYMIGYVVFDF
SEQ ID NO: 114-Heavy chain variable region
QVQLVQSGAEMKKPGASVTVSCKASGYTFTNYYIQWVRQAPGQGLEWMGVINPYDGSTNYAQRFQGRV
TLTRDTTTTTVNMELSGLRSEDTAVYYCARDYMIGYVVFDFWGQGTLVTVSS
SEQ ID NO: 115-Heavy chain CDR1
NYYIQ
SEQ ID NO: 116-Heavy chain CDR2
VINPYDGSTNYAQRFQG
SEQ ID NO: 117-Heavy chain CDR3
DYMIGYVVFDF
SEQ ID NO: 118-Heavy chain variable region
QVQLVQSGAEVKKPGASVKVSCKASGYTFTNYYIQWVRQAPGQGLEWMGVINPYDGSTNYAQRFQGKIT
LTRDTSTTTVYMELSSLGSEDTAVYYCARDYMIGYVVFDYWGQGTMVTVSS
SEQ ID NO: 119-Heavy chain CDR1
NYYIQ
SEQ ID NO: 120-Heavy chain CDR2
VINPYDGSTNYAQRFQG
SEQ ID NO: 121-Heavy chain CDR3
DYMIGYVVFDY
SEQ ID NO: 122-Heavy chain variable region
EVQLLESGAEVKKPGASVKVSCKASGYTFNRYYIHWVRQAPGQGLEWMGVINPYDGSTDYAQKFRDRIT
MTRDTSTNTVYMDLSSLRSEDTAVYFCARDAMEGDSYFDNWGRGTLVTVSS
SEQ ID NO: 123-Heavy chain CDR1
RYYIH
SEQ ID NO: 124-Heavy chain CDR2
VINPYDGSTDYAQKFRD
SEQ ID NO: 125-Heavy chain CDR3
DAMEGDSYFDN
SEQ ID NO: 126-Heavy chain variable region
QVQLVQSGAEVKKPGASVKVSCKASGYTFNRYYLHWVRQAPGQGLEWMGVINPYDGSTNYAQKFRGRV
TMTRDTSTNTVYMELSSLRSEDTAVYFCARDAMEGDSYFDNWGQGTTVTVSS
SEQ ID NO: 127-Heavy chain CDR1
RYYLH
SEQ ID NO: 128-Heavy chain CDR2
VINPYDGSTNYAQKFRG
SEQ ID NO: 129-Heavy chain CDR3
DAMEGDSYFDN
SEQ ID NO: 130-Heavy chain variable region
QVQLVQSGAEVKKPGASVRVSCKASGYTFINYYLQWVRQAPGQGLEWMGTINPFDGSTHYAQKFQGRVT
LTRDTSTSTVYMELNSLRSDDTAVYYCARDYMIGYVLFDYWGQGTLVTVSS
SEQ ID NO: 131-Heavy chain CDR1
NYYLQ
SEQ ID NO: 132-Heavy chain CDR2
TINPFDGSTHYAQKFQG
SEQ ID NO: 133-Heavy chain CDR3
DYMIGYVLFDY
SEQ ID NO: 134-Heavy chain variable region
QVQLVQSGAEVKKPGASVEVSCKASGYTFINYYIHWVRQAPGQGLEWMGVINPFDGSTNYAQKFQGRVT
VTRDTSTSTVFLDLSSLRPEDTAVYYCARDSMIGYVLFDYWGQGTLVTVSS
SEQ ID NO: 135-Heavy chain CDR1
NYYIH
SEQ ID NO: 136-Heavy chain CDR2
VINPFDGSTNYAQKFQG
SEQ ID NO: 137-Heavy chain CDR3
DSMIGYVLFDY
SEQ ID NO: 138-Heavy chain variable region
QVQLVQSGAEVKKPGASVRVSCKASGYTFINYYLQWVRQAPGQGLEWMGTINPFDGSTHYAQKFQGRVT
MTRDTSTSTVYMELSSLRSEDTAVYYCARDSMIGYVLFDYWGQGTLVTVSS
SEQ ID NO: 139-Heavy chain CDR1
NYYLQ
SEQ ID NO: 140-Heavy chain CDR2
TINPFDGSTHYAQKFQG
SEQ ID NO: 141-Heavy chain CDR3
DSMIGYVLFDY
SEQ ID NO: 142-Heavy chain variable region
QVQLVQSGAEVKKPGASVMLSCKASGYIFTSYYIQWVRQAPGQGLEWMGVVNPYDGSTNYAQKFQGRV
SITRDTSTSTVFMELNSLRSEDTAVYYCARDSMLGHVIFDFWGQGTLVTVSS
SEQ ID NO: 143-Heavy chain CDR1
SYYIQ
SEQ ID NO: 144-Heavy chain CDR2
VVNPYDGSTNYAQKFQG
SEQ ID NO: 145-Heavy chain CDR3
DSMLGHVIFDF
SEQ ID NO: 146-Heavy chain variable region
QVQLVQSGAEVKKPGASVKVSCKASGYAFTSYYIHWVRQAPGQGLEWMGVINPYGGSTNYAQKFQGRV
TMTRDTSTRIVYMDLSSLRSEDTAVYYCARDAMEGDSYFDYWGQGTLVTVSS
SEQ ID NO: 147-Heavy chain CDR1
SYYIH
SEQ ID NO: 148-Heavy chain CDR2
VINPYGGSTNYAQKFQG
SEQ ID NO: 149-Heavy chain CDR3
DAMEGDSYFDY
SEQ ID NO: 150-Heavy chain variable region
QVQLVQSGAEVKTPGASVKVSCKASGYTFTNYYIQWVRQAPGQALEWMGVINPYDGSTHYAQKFQGRV
TLTRDTSTTTVYMDLSSLRSEDAAVYYCARDYMLGYVVFDYWGQGTLVTVSS
SEQ ID NO: 151-Heavy chain CDR1
NYYIQ
SEQ ID NO: 152-Heavy chain CDR2
VINPYDGSTHYAQKFQG
SEQ ID NO: 153-Heavy chain CDR3
DYMLGYVVFDY
SEQ ID NO: 154-Heavy chain variable region
QVQLVQSGAEVKKSGASVKVSCKASGYIFTSYYIQWVRQAPGQGLEWMGVINPFDGSTDYAQKFQGRVT
VTRDTSTTTVFMELSSLRSEDTAVYYCARDNMLGYVLFDYWGQGTLVTVSS
SEQ ID NO: 155-Heavy chain CDR1
SYYIQ
SEQ ID NO: 156-Heavy chain CDR2
VINPFDGSTDYAQKFQG
SEQ ID NO: 157-Heavy chain CDR3
DNMLGYVLFDY
SEQ ID NO: 158-Heavy chain variable region
QVQLVQSGAEVKKPGASVKVSCKASGYTFTSYYMHWVRQAPGQGLEWMGVINPFDGSTNYAQKFQGRV
TVTRDTATTTVFMDLSSLRSEDTAVYYCARDNMLGYVLFDYWGQGTLVTVSS
SEQ ID NO: 159-Heavy chain CDR1
SYYMH
SEQ ID NO: 160-Heavy chain CDR2
VINPFDGSTNYAQKFQG
SEQ ID NO: 161-Heavy chain CDR3
DNMLGYVLFDY
SEQ ID NO: 162-Heavy chain variable region
QVQLVQSGAEVKKPGASVMLSCKASGYIFTSYYIQWVRQAPGQGLEWMGVVNPYDGSTNYAQKFQGRV
TITRDTSTSTVFMELSSLRSEDTAVYYCARDNMLGHVIFDFWGQGTLVTVSS
SEQ ID NO: 163-Heavy chain CDR1
SYYIQ
SEQ ID NO: 164-Heavy chain CDR2
VVNPYDGSTNYAQKFQG
SEQ ID NO: 165-Heavy chain CDR3
DNMLGHVIFDF
SEQ ID NO: 166-Heavy chain variable region
QVQLVESGAEVKKPGASVKVSCKASGYTFNRYYIHWVRQAPGQGLEWMGVINPSDGSTNYAQKFRGRIT
MTRDTSTNIVYMELSSLRSEDTAVYFCARDAMGGDSYFDNWGQGTLVTVSS
SEQ ID NO: 167-Heavy chain CDR1
RYYIH
SEQ ID NO: 168-Heavy chain CDR2
VINPSDGSTNYAQKFRG
SEQ ID NO: 169-Heavy chain CDR3
DAMGGDSYFDN
SEQ ID NO: 170-Heavy chain variable region
QVQLVQSGAEVKKPGASVQVSCKASGYTFINYYLQWVRQAPGQGLEWMGTINPFDGSTHYAQKFQGRVT
MTRDTSTNTVYMELNSLRSEDTAVYYCARDRMIGYVLFDYWGQGTLVTVSS
SEQ ID NO: 171-Heavy chain CDR1
NYYLQ
SEQ ID NO: 172-Heavy chain CDR2
TINPFDGSTHYAQKFQG
SEQ ID NO: 173-Heavy chain CDR3
DRMIGYVLFDY
SEQ ID NO: 174-Heavy chain variable region
EVQLVESGAEVKKPGASVKVSCKASGYTFNRYYLHWVRQAPGQGLEWMGVINPYDGSTNYAQKFRGRIT
MTRDTSTNTVYMELSSLRSEDTAVYFCARDAMEGDSYFDKWGQGTLVTVSS
SEQ ID NO: 175-Heavy chain CDR1
RYYLH
SEQ ID NO: 176-Heavy chain CDR2
VINPYDGSTNYAQKFRG
SEQ ID NO: 177-Heavy chain CDR3
DAMEGDSYFDK
SEQ ID NO: 178-Heavy chain variable region
QVQLVQSGAEVKKPGASVKVSCKASGYTFNRHYIQWVRQAPGQGLEWMGTINPYDGSTFYAQKFQDRVT
MTRDTSTSTVYMDLNSLKSEDTAVYYCARDYMRGHVLFDYWGQGTLVTVSS
SEQ ID NO: 179-Heavy chain CDR1
RHYIQ
SEQ ID NO: 180-Heavy chain CDR2
TINPYDGSTFYAQKFQD
SEQ ID NO: 181-Heavy chain CDR3
DYMRGHVLFDY
SEQ ID NO: 182-Heavy chain variable region
QVQLVQSGAEVKKPGASVKVSCKASGYSFTYSYIQWVRLAPGRGLEWMGVINPYDGSTDYAQKFQGRVT
LTRDTSTTTVYMELSSLTSEDTAVYYCARDFMIGYVVFDYWGQGTLVTVSS
SEQ ID NO: 183-Heavy chain CDR1
YSYIQ
SEQ ID NO: 184-Heavy chain CDR2
VINPYDGSTDYAQKFQG
SEQ ID NO: 185-Heavy chain CDR3
DFMIGYVVFDY
SEQ ID NO: 186-Heavy chain variable region
QVQLVQSGAEVKKPGASVKVSCKASGYTFNRYYIHWVRQAPGQGLEWMGVINPYDGSTNYAQKFRGRIT
MTRDTSTNTVYMDLSSLRSEDTAVYFCARDAMEGDSYLDNWGRGTLVTVSS
SEQ ID NO: 187-Heavy chain CDR1
RYYIH
SEQ ID NO: 188-Heavy chain CDR2
VINPYDGSTNYAQKFRG
SEQ ID NO: 189-Heavy chain CDR3
DAMEGDSYLDN
SEQ ID NO: 190-Heavy chain variable region
QVQLVQSGAEVKKPGASVKVSCKASGYAFTNYYIHWVRQAPGHGLTWMGVINPFDSSTDFAQKFQGRLT
MTRDTSTSTVYMDLSSLRSEDTAVYYCARDYMRGHVVFDNWGQGTLVTVSS
SEQ ID NO: 191-Heavy chain CDR1
NYYIH
SEQ ID NO: 192-Heavy chain CDR2
VINPFDSSTDFAQKFQG
SEQ ID NO: 193-Heavy chain CDR3
DYMRGHVVFDN
SEQ ID NO: 194-Heavy chain variable region
QVQLVQSGAEVKKPGTSVKVSCTASGYTFTNYYIQWVRQAPGQGLEWLGVINPYDGSTDYAQKFQGRVT
LTRDTSTTTVYMELSSLRSEDTAVYYCARDYMLGYVVFDFWGQGTLVTVSS
SEQ ID NO: 195-Heavy chain CDR1
NYYIQ
SEQ ID NO: 196-Heavy chain CDR2
VINPYDGSTDYAQKFQG
SEQ ID NO: 197-Heavy chain CDR3
DYMLGYVVFDF
SEQ ID NO: 198-Heavy chain variable region
QVQLVQSGAEVKKPGASVKVSCKASGYTFINNYIQWVRQAPGQGLEWMGVINPYDGSIHYAQKFQGRVT
LTRDTSTTTVYMELSSLRSEDTAVYYCARDYMIGYVVFEYWGQGTLVTVSS
SEQ ID NO: 199-Heavy chain CDR1
NNYIQ
SEQ ID NO: 200-Heavy chain CDR2
VINPYDGSIHYAQKFQG
SEQ ID NO: 201-Heavy chain CDR3
DYMIGYVVFEY
SEQ ID NO: 202-Heavy chain variable region
QVQLVQSGAEVKNPGASVKFSCKASGYTFINYYLQWLRQAPGQGLEWMGTINPFDGSTSYAQNFQGRVT
MTRDTSSTTVYMELNSLKSEDTAVYYCARDSMIGYVLFDSWGQGTLVTVSS
SEQ ID NO: 203-Heavy chain CDR1
NYYLQ
SEQ ID NO: 204-Heavy chain CDR2
TINPFDGSTSYAQNFQG
SEQ ID NO: 205-Heavy chain CDR3
DSMIGYVLFDS
SEQ ID NO: 206-Heavy chain variable region
QVQLVQSGAEVKKPGASVRVSCKASGYPFINYYIQWVRQAPGQGLEWMGVINPFDGSTNYAQKFQGRVT
VTRDTSTSTVFMDLNSLRSEDTAVYYCARDSMIGYVLFDFWGQGTLVTVSS
SEQ ID NO: 207-Heavy chain CDR1
NYYIQ
SEQ ID NO: 208-Heavy chain CDR2
VINPFDGSTNYAQKFQG
SEQ ID NO: 209-Heavy chain CDR3
DSMIGYVLFDF
SEQ ID NO: 210-Heavy chain 5E5 analog
QVQLVQSGAEVKKTGSSVKVSCKASGYTFTDHAIHWVRQAPGQALEWMGHFSPGNTDIKYNDKFKGRVT
LTVDRSMSTAYMELSSLRSEDTAMYYCKTSTFFFDYWGQGTMVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD
KRVEPKSCDKTHTCPPCPAPELGRGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVF
SCSVMHEALHNHYTQKSLSLSPGK
SEQ ID NO: 211-Light chain 5E5 analog
DIVMTQSPDSLAVSLGERATINCKSSQSLLNSGDQKNYLTWYQQKPGQPPKLLIYWASTRESGVPDRFSGS
GSGTDFTLTISSLQAEDVAVYYCQNDYSYPLTFGQGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPVTK
SFNRGEC
SEQ ID NO: 212-Heavy chain pankomab analog
EVKLVESGGGLVQPGGSMKLSCVASGFTFSNYWMNWVRQSPEKGLEWVAEIRLKSNNYTTHYAESVKGR
FTISRDDSKSSVSLQMNNLRVEDTGIYYCTRHYYFDYWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD
KRVEPKSCDKTHTCPPCPAPELGRGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNVF
SCSVMHEALHNHYTQKSLSLSPGK
SEQ ID NO: 213-Light chain pankomab analog
DIVMTQAAFSNPVTLGTSASISCRSSKSLLHSNGITYFFWYLQKPGLSPQLLIYQMSNLASGVPDRFSSSGSG
TDFTLRISRVEAEDVGVYYCAQNLELPPTFGGGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSFNR
GEC
SEQ ID NO: 214-Heavy chain HT186-D11 analog
QMQLVQSEAELKKPGASVKVSCKASGYSFTGHYMHWVRQAPGQGLEWMGWIDPVTGGTKYAQNFQGW
VTMTRDTSIRTAYLELSRLRSDDTAMYYCAREVTGDRGQFDKWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPS
NTKVDKRVEPKSCDKTHTCPPCPAPELGRGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 215-Light chain HT186-D11 analog
QSVLTQPPSVSVAPGKTARITCGGNNIGSKSVHWYQQKPGQAPALVIYYGSNRPSGIPERFSGSNSGNTATL
TISRVEAGDEADYYCQVWDSSSDWVFGGGTKLEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRGE
C
SEQ ID NO: 216-Heavy chain isotype control IgG
EVQLVETGAEVKKPGASVKVSCKASDYIFTKYDINWVRQAPGQGLEWMGWMSANTGNTGYAQKFQGRV
TMTRDTSINTAYMELSSLTSGDTAVYFCARSSLFKTETAPYYHFALDVWGQGTTVTVSSASTKGPSVFPLA
PSSKSTSGGTAALGCLVKDYFPEPVTVSWNSGALTSGVHTFPAVLQSSGLYSLSSVVTVPSSSLGTQTYICN
VNHKPSNTKVDKRVEPKSCDKTHTCPPCPAPELGRGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 217-Light chain isotype control IgG
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
SEQ ID NO: 218-Light chain variable region
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPTFGQGTKVEIK
SEQ ID NO: 219-Light chain CDR1 according to IMGT
QSISSY
SEQ ID NO: 220-Light chain CDR2 according to IMGT
AAS
SEQ ID NO: 221-Light chain CDR3 according to IMGT
QQSYSTPPT
SEQ ID NO: 222-hinge region
EPKSCDKTHTCPPCP
SEQ ID NO: 223-CH1 region
ASTKGPSVFPLAPSSKSTSGGTAALGCLVKDYFPEPVTVSWNSGALTSGVHTFPAVLQSSGLYSLSSVVTVP
SSSLGTQTYICNVNHKPSNTKVDKRV
SEQ ID NO: 224-CH2 region
APELLGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAK
SEQ ID NO: 225-CH3 region
GQPREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPG
SEQ ID NO: 226-CL region
RTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 227-V region VK1-39
DIQMTQSPSSLSASVGDRVTITCRASQSISSYLNWYQQKPGKAPKLLIYAASSLQSGVPSRFSGSGSGTDFTL
TISSLQPEDFATYYCQQSYSTP
SEQ ID NO: 228-VK1-39/JK1
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPTFGQGTKVEIK
SEQ ID NO: 229-VK1-39/JK5
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
SEQ ID NO: 230-Light chain CDR1 according to IMGT
QSISSY
SEQ ID NO: 231-Light chain CDR2 according to IMGT
AAS
SEQ ID NO: 232-Light chain CDR3 according to IMGT
QQSYSTPPIT
SEQ ID NO: 233-V region VK3-15
EIVMTQSPATLSVSPGERATLSCRASQSVSSNLAWYQQKPGQAPRLLIYGASTRATGIPARFSGSGSGTEFTL
TISSLQSEDFAVYYCQQYNNWP
SEQ ID NO: 234-VK3-15/JK1
EIVMTQSPATLSVSPGERATLSCRASQSVSSNLAWYQQKPGQAPRLLIYGASTRATGIPARFSGSGSGTEFTL
TISSLQSEDFAVYYCQQYNNWPWTFGQGTKVEIK
SEQ ID NO: 235-Light chain CDR1 according to IMGT
QSVSSN
SEQ ID NO: 236-Light chain CDR2 according to IMGT
GAS
SEQ ID NO: 237-Light chain CDR3 according to IMGT
QQYNNWPWT
SEQ ID NO: 238-V region VK3-20
EIVLTQSPGTLSLSPGERATLSCRASQSVSSSYLAWYQQKPGQAPRLLIYGASSRATGIPDRFSGSGSGTDFT
LTISRLEPEDFAVYYCQQYGSSP
SEQ ID NO: 239-VK3-20/JK1
EIVLTQSPGTLSLSPGERATLSCRASQSVSSSYLAWYQQKPGQAPRLLIYGASSRATGIPDRFSGSGSGTDFT
LTISRLEPEDFAVYYCQQYGSSPWTFGQGTKVEIK
SEQ ID NO: 240-Light chain CDR1 according to IMGT
QSVSSSY
SEQ ID NO: 241-Light chain CDR2 according to IMGT
GAS
SEQ ID NO: 242-Light chain CDR3 according to IMGT
QQYGSSPWT
SEQ ID NO: 243-V region VL3-21
SYVLTQPPSVSVAPGETARITCGGDNIGRKSVYWYQQKSGQAPVLVIYYDSDRPSGIPERFSGSNSGNTATL
TISRVEAGDEADYYCQVWDGSSDH
SEQ ID NO: 244-VL3-21/JL3
SYVLTQPPSVSVAPGETARITCGGDNIGRKSVYWYQQKSGQAPVLVIYYDSDRPSGIPERFSGSNSGNTATL
TISRVEAGDEADYYCQVWDGSSDHWVFGGGTKLTVL
SEQ ID NO: 245-Light chain CDR1 according to IMGT
NIGRKS
SEQ ID NO: 246-Light chain CDR2 according to IMGT
YDS
SEQ ID NO: 247-Light chain CDR3 according to IMGT
QVWDGSSDHWV
SEQ ID NO: 248-Light chain CDR3 according to IMGT
QQSYSTPPT
SEQ ID NO: 249
PAPGSTAPPAHGVTSAPDTRPAPG

1. A binding domain that binds a MUC1 peptide comprising the amino acid sequence of SEQ ID NO: 249, wherein the threonine (T) residue at position 14 of the MUC1 peptide comprises α-GalNAc glycosylation or Neu5Ac-α(2-6)-GalNAc glycosylation.

2. The binding domain according to claim 1, wherein the threonine (T) residue at position 14 of the MUC1 peptide comprises α-GalNAc glycosylation.

3. The binding domain according to claim 1, wherein the threonine (T) residue at position 14 of the MUC1 peptide comprises Neu5Ac-α(2-6)-GalNAc glycosylation.

4. The binding domain according to claim 1, wherein in addition the serine (S) residue at position 15 of the MUC1 peptide comprises α-GalNAc glycosylation or Neu5Ac-α(2-6)-GalNAc glycosylation.

5. The binding domain according to claim 4, wherein the serine (S) residue at position 15 of the MUC1 peptide comprises α-GalNAc glycosylation.

6. The binding domain according to claim 4, wherein the serine (S) residue at position 15 of the MUC1 peptide comprises Neu5Ac-α(2-6)-GalNAc glycosylation.

7. The binding domain according to claim 1, which does not bind to a MUC1 peptide comprising the amino acid sequence of SEQ ID NO: 249 not comprising α-GalNAc glycosylation or Neu5Ac-α(2-6)-GalNAc glycosylation.

8. The binding domain according to claim 1, wherein the binding to the MUC-1 peptide is determined in a glycopeptide array.

9. A binding domain that specifically binds to cancer-associated MUC-1, wherein the binding domain binds to an epitope on MUC1 comprising the VTSA motif of the N-terminal domain of MUC1, wherein the threonine (T) residue is α-GalNAc glycosylated or Neu5Ac-α(2-6)-GalNAc glycosylated.

10. The binding domain according to claim 9, wherein the threonine (T) is α-GalNAc glycosylated.

11. The binding domain according to claim 9, wherein the threonine (T) is Neu5Ac-α(2-6)-GalNAc glycosylated.

12. The binding domain according to claim 9, wherein in addition the serine (S) residue is α-GalNAc glycosylated or Neu5Ac-α(2-6)-GalNAc glycosylated.

13. The binding domain according to claim 12, wherein the serine (S) residue is α-GalNAc glycosylated.

14. The binding domain according to claim 12, wherein the serine (S) residue is Neu5Ac-α(2-6)-GalNAc glycosylated.

15. The binding domain according to claim 9, wherein the binding to the epitope on MUC1 is determined in a glycopeptide array.

16. A binding domain having binding specificity for cancer-associated MUC1, wherein the binding domain comprises a heavy chain variable region comprising:
a) heavy chain CDR1 (HCDR1), heavy chain CDR2 (HCDR2), and heavy chain CDR3 (HCDR3), having the amino acid sequence of SEQ ID NO: 2, SEQ ID NO: 3, and SEQ ID NO: 4, respectively;
b) heavy chain CDR1 (HCDR1), heavy chain CDR2 (HCDR2), and heavy chain CDR3 (HCDR3), having the amino acid sequence of SEQ ID NO: 6, SEQ ID NO: 7, and SEQ ID NO: 8, respectively;
c) heavy chain CDR1 (HCDR1), heavy chain CDR2 (HCDR2), and heavy chain CDR3 (HCDR3), having the amino acid sequence of SEQ ID NO: 10, SEQ ID NO: 11, and SEQ ID NO: 12, respectively;
d) heavy chain CDR1 (HCDR1), heavy chain CDR2 (HCDR2), and heavy chain CDR3 (HCDR3), having the amino acid sequence of SEQ ID NO: 14, SEQ ID NO: 15, and SEQ ID NO: 16, respectively;
e) heavy chain CDR1 (HCDR1), heavy chain CDR2 (HCDR2), and heavy chain CDR3 (HCDR3), having the amino acid sequence of SEQ ID NO: 18, SEQ ID NO: 19, and SEQ ID NO: 20, respectively;
f) heavy chain CDR1 (HCDR1), heavy chain CDR2 (HCDR2), and heavy chain CDR3 (HCDR3), having the amino acid sequence of SEQ ID NO: 22, SEQ ID NO: 23, and SEQ ID NO: 24, respectively;
g) heavy chain CDR1 (HCDR1), heavy chain CDR2 (HCDR2), and heavy chain CDR3 (HCDR3), having the amino acid sequence of SEQ ID NO: 26, SEQ ID NO: 27, and SEQ ID NO: 28, respectively;
h) heavy chain CDR1 (HCDR1), heavy chain CDR2 (HCDR2), and heavy chain CDR3 (HCDR3), having the amino acid sequence of SEQ ID NO: 30, SEQ ID NO: 31, and SEQ ID NO: 32, respectively;
i) heavy chain CDR1 (HCDR1), heavy chain CDR2 (HCDR2), and heavy chain CDR3 (HCDR3), having the amino acid sequence of SEQ ID NO: 34, SEQ ID NO: 35, and SEQ ID NO: 36, respectively;
j) heavy chain CDR1 (HCDR1), heavy chain CDR2 (HCDR2), and heavy chain CDR3 (HCDR3), having the amino acid sequence of SEQ ID NO: 38, SEQ ID NO: 39, and SEQ ID NO: 40, respectively;
k) heavy chain CDR1 (HCDR1), heavy chain CDR2 (HCDR2), and heavy chain CDR3 (HCDR3), having the amino acid sequence of SEQ ID NO: 42, SEQ ID NO: 43, and SEQ ID NO: 44, respectively;
l) heavy chain CDR1 (HCDR1), heavy chain CDR2 (HCDR2), and heavy chain CDR3 (HCDR3), having the amino acid sequence of SEQ ID NO: 46, SEQ ID NO: 47, and SEQ ID NO: 48, respectively;
m) heavy chain CDR1 (HCDR1), heavy chain CDR2 (HCDR2), and heavy chain CDR3 (HCDR3), having the amino acid sequence of SEQ ID NO: 50, SEQ ID NO: 51, and SEQ ID NO: 52, respectively;
n) heavy chain CDR1 (HCDR1), heavy chain CDR2 (HCDR2), and heavy chain CDR3 (HCDR3), having the amino acid sequence of SEQ ID NO: 54, SEQ ID NO: 55, and SEQ ID NO: 56, respectively;
o) heavy chain CDR1 (HCDR1), heavy chain CDR2 (HCDR2), and heavy chain CDR3 (HCDR3), having the amino acid sequence of SEQ ID NO: 58, SEQ ID NO: 60, and SEQ ID NO: 61, respectively;
p) heavy chain CDR1 (HCDR1), heavy chain CDR2 (HCDR2), and heavy chain CDR3 (HCDR3), having the amino acid sequence of SEQ ID NO: 63, SEQ ID NO: 64, and SEQ ID NO: 65, respectively;
q) heavy chain CDR1 (HCDR1), heavy chain CDR2 (HCDR2), and heavy chain CDR3 (HCDR3), having the amino acid sequence of SEQ ID NO: 67, SEQ ID NO: 68, and SEQ ID NO: 69, respectively;
r) heavy chain CDR1 (HCDR1), heavy chain CDR2 (HCDR2), and heavy chain CDR3 (HCDR3), having the amino acid sequence of SEQ ID NO: 71, SEQ ID NO: 72, and SEQ ID NO: 73, respectively;
s) heavy chain CDR1 (HCDR1), heavy chain CDR2 (HCDR2), and heavy chain CDR3 (HCDR3), having the amino acid sequence of SEQ ID NO: 75, SEQ ID NO: 76, and SEQ ID NO: 77, respectively;
t) heavy chain CDR1 (HCDR1), heavy chain CDR2 (HCDR2), and heavy chain CDR3 (HCDR3), having the amino acid sequence of SEQ ID NO: 79, SEQ ID NO: 80, and SEQ ID NO: 81, respectively;
u) heavy chain CDR1 (HCDR1), heavy chain CDR2 (HCDR2), and heavy chain CDR3 (HCDR3), having the amino acid sequence of SEQ ID NO: 83, SEQ ID NO: 84, and SEQ ID NO: 85, respectively;
v) heavy chain CDR1 (HCDR1), heavy chain CDR2 (HCDR2), and heavy chain CDR3 (HCDR3), having the amino acid sequence of SEQ ID NO: 87, SEQ ID NO: 88, and SEQ ID NO: 89, respectively;
w) heavy chain CDR1 (HCDR1), heavy chain CDR2 (HCDR2), and heavy chain CDR3 (HCDR3), having the amino acid sequence of SEQ ID NO: 91, SEQ ID NO: 92, and SEQ ID NO: 93, respectively;
x) heavy chain CDR1 (HCDR1), heavy chain CDR2 (HCDR2), and heavy chain CDR3 (HCDR3), having the amino acid sequence of SEQ ID NO: 95, SEQ ID NO: 96, and SEQ ID NO: 97, respectively;
y) heavy chain CDR1 (HCDR1), heavy chain CDR2 (HCDR2), and heavy chain CDR3 (HCDR3), having the amino acid sequence of SEQ ID NO: 99, SEQ ID NO: 100, and SEQ ID NO: 101, respectively;
z) heavy chain CDR1 (HCDR1), heavy chain CDR2 (HCDR2), and heavy chain CDR3 (HCDR3), having the amino acid sequence of SEQ ID NO: 103, SEQ ID NO: 104, and SEQ ID NO: 105, respectively;
aa) heavy chain CDR1 (HCDR1), heavy chain CDR2 (HCDR2), and heavy chain CDR3 (HCDR3), having the amino acid sequence of SEQ ID NO: 107, SEQ ID NO: 108, and SEQ ID NO: 109, respectively;
bb) heavy chain CDR1 (HCDR1), heavy chain CDR2 (HCDR2), and heavy chain CDR3 (HCDR3), having the amino acid sequence of SEQ ID NO: 111, SEQ ID NO: 112, and SEQ ID NO: 113, respectively;
cc) heavy chain CDR1 (HCDR1), heavy chain CDR2 (HCDR2), and heavy chain CDR3 (HCDR3), having the amino acid sequence of SEQ ID NO: 115, SEQ ID NO: 116, and SEQ ID NO: 117, respectively;
dd) heavy chain CDR1 (HCDR1), heavy chain CDR2 (HCDR2), and heavy chain CDR3 (HCDR3), having the amino acid sequence of SEQ ID NO: 119, SEQ ID NO: 120, and SEQ ID NO: 121, respectively;
ee) heavy chain CDR1 (HCDR1), heavy chain CDR2 (HCDR2), and heavy chain CDR3 (HCDR3), having the amino acid sequence of SEQ ID NO: 123, SEQ ID NO: 124, and SEQ ID NO: 125, respectively;
ff) heavy chain CDR1 (HCDR1), heavy chain CDR2 (HCDR2), and heavy chain CDR3 (HCDR3), having the amino acid sequence of SEQ ID NO: 127, SEQ ID NO: 128, and SEQ ID NO: 129, respectively;
gg) heavy chain CDR1 (HCDR1), heavy chain CDR2 (HCDR2), and heavy chain CDR3 (HCDR3), having the amino acid sequence of SEQ ID NO: 131, SEQ ID NO: 132, and SEQ ID NO: 133, respectively;
hh) heavy chain CDR1 (HCDR1), heavy chain CDR2 (HCDR2), and heavy chain CDR3 (HCDR3), having the amino acid sequence of SEQ ID NO: 135, SEQ ID NO: 136, and SEQ ID NO: 137, respectively;
ii) heavy chain CDR1 (HCDR1), heavy chain CDR2 (HCDR2), and heavy chain CDR3 (HCDR3), having the amino acid sequence of SEQ ID NO: 139, SEQ ID NO: 140, and SEQ ID NO: 141, respectively;
jj) heavy chain CDR1 (HCDR1), heavy chain CDR2 (HCDR2), and heavy chain CDR3 (HCDR3), having then-amino acid sequence of SEQ ID NO: 143, SEQ ID NO: 144, and SEQ ID NO: 145, respectively;
kk) heavy chain CDR1 (HCDR1), heavy chain CDR2 (HCDR2), and heavy chain CDR3 (HCDR3), having then-amino acid sequence of SEQ ID NO: 147, SEQ ID NO: 148, and SEQ ID NO: 149, respectively;
ll) heavy chain CDR1 (HCDR1), heavy chain CDR2 (HCDR2), and heavy chain CDR3 (HCDR3), having the amino acid sequence of SEQ ID NO: 151, SEQ ID NO: 152, and SEQ ID NO: 153, respectively;
mm) heavy chain CDR1 (HCDR1), heavy chain CDR2 (HCDR2), and heavy chain CDR3 (HCDR3), having the amino acid sequence of SEQ ID NO: 155, SEQ ID NO: 156, and SEQ ID NO: 157, respectively;
nn) heavy chain CDR1 (HCDR1), heavy chain CDR2 (HCDR2), and heavy chain CDR3 (HCDR3), having the amino acid sequence of SEQ ID NO: 159, SEQ ID NO: 160, and SEQ ID NO: 161, respectively;
oo) heavy chain CDR1 (HCDR1), heavy chain CDR2 (HCDR2), and heavy chain CDR3 (HCDR3), having the amino acid sequence of SEQ ID NO: 163, SEQ ID NO: 164, and SEQ ID NO: 165, respectively;
pp) heavy chain CDR1 (HCDR1), heavy chain CDR2 (HCDR2), and heavy chain CDR3 (HCDR3), having the amino acid sequence of SEQ ID NO: 167, SEQ ID NO: 168, and SEQ ID NO: 169, respectively;
qq) heavy chain CDR1 (HCDR1), heavy chain CDR2 (HCDR2), and heavy chain CDR3 (HCDR3), having the amino acid sequence of SEQ ID NO: 171, SEQ ID NO: 172, and SEQ ID NO: 173, respectively;
rr) heavy chain CDR1 (HCDR1), heavy chain CDR2 (HCDR2), and heavy chain CDR3 (HCDR3), having the amino acid sequence of SEQ ID NO: 175, SEQ ID NO: 176, and SEQ ID NO: 177, respectively;
ss) heavy chain CDR1 (HCDR1), heavy chain CDR2 (HCDR2), and heavy chain CDR3 (HCDR3), having the amino acid sequence of SEQ ID NO: 179, SEQ ID NO: 180, and SEQ ID NO: 181, respectively;
tt) heavy chain CDR1 (HCDR1), heavy chain CDR2 (HCDR2), and heavy chain CDR3 (HCDR3), having then-amino acid sequence of SEQ ID NO: 183, SEQ ID NO: 184, and SEQ ID NO: 185, respectively;
uu) heavy chain CDR1 (HCDR1), heavy chain CDR2 (HCDR2), and heavy chain CDR3 (HCDR3), having then-amino acid sequence of SEQ ID NO: 187, SEQ ID NO: 188, and SEQ ID NO: 189, respectively;
vv) heavy chain CDR1 (HCDR1), heavy chain CDR2 (HCDR2), and heavy chain CDR3 (HCDR3), having the amino acid sequence of SEQ ID NO: 191, SEQ ID NO: 192, and SEQ ID NO: 193, respectively;
ww) heavy chain CDR1 (HCDR1), heavy chain CDR2 (HCDR2), and heavy chain CDR3 (HCDR3), having the amino acid sequence of SEQ ID NO: 195, SEQ ID NO: 196, and SEQ ID NO: 197, respectively;
xx) heavy chain CDR1 (HCDR1), heavy chain CDR2 (HCDR2), and heavy chain CDR3 (HCDR3), having the amino acid sequence of SEQ ID NO: 199, SEQ ID NO: 200, and SEQ ID NO: 201, respectively;
yy) heavy chain CDR1 (HCDR1), heavy chain CDR2 (HCDR2), and heavy chain CDR3 (HCDR3), having the amino acid sequence of SEQ ID NO: 203, SEQ ID NO: 204, and SEQ ID NO: 205, respectively; or
zz) heavy chain CDR1 (HCDR1), heavy chain CDR2 (HCDR2), and heavy chain CDR3 (HCDR3), having the amino acid sequence of SEQ ID NO: 207, SEQ ID NO: 208, and SEQ ID NO: 209, respectively;
wherein each of the HCDRs may comprise at most three, two, or one amino acid substitutions.

17. The binding domain according to claim 16, wherein the binding domain comprises a heavy chain variable region having an amino acid sequence as set forth in any one of SEQ ID NO: 1; 5; 9; 13; 17; 21; 25; 29; 33; 37; 41; 45; 49; 53; 57; 62; 66; 70; 74; 78; 82; 86; 90; 94; 98; 102; 106; 110; 114; 118; 122; 126; 130; 134; 138; 142; 146; 150; 154; 158; 162; 166; 170; 174; 178; 182; 186; 190; 194; 198; 202; or 206, or having at least 80% sequence identity thereto.

18. The binding domain according to claim 16, wherein the binding domain comprises a light chain variable region comprising light chain CDR1 (LCDR1), light chain CDR2 (LCDR2), and light chain CDR3 (LCDR3), having the amino acid sequence of SEQ ID NO: 219, SEQ ID NO: 220, and SEQ ID NO: 221, respectively, wherein each of the LCDRs may comprise at most three, two, or one amino acid substitutions.

19. The binding domain according to claim 16, wherein the binding domain comprises a light chain variable region having the amino acid sequence of SEQ ID NO: 218, or having at least 80% sequence identity thereto.

20. The binding domain according to claim 1, wherein the binding domain is a Fab.

21. A binding moiety comprising two binding domains according to claim 1.

22. The binding moiety according to claim 21, wherein the binding moiety is an IgG1 or IgG4 antibody.

23. The binding moiety according to claim 21, wherein the binding of the binding moiety to an Fc receptor is eliminated or reduced.

24. The binding moiety according to claim 21, wherein the binding of the binding moiety to an Fc receptor is enhanced.

25. The binding moiety according to claim 21, wherein the binding moiety shows a higher binding signal than a reference antibody comprising two heavy chains having the amino acid sequence of SEQ ID NO: 210 and a light chain having the amino acid sequence of SEQ ID NO: 211.

26. A pharmaceutical composition comprising an effective amount of a binding domain according to claim 1, and a pharmaceutically acceptable carrier.

27-28. (canceled)

29. A method for treating a disease, comprising administering an effective amount of a binding domain according to claim 1 to an individual in need thereof.

30. A method for treating cancer, comprising administering an effective amount of a binding domain according to claim 1 to an individual in need thereof.

31. A nucleic acid comprising a nucleic acid sequence encoding a heavy chain variable region as defined in claim 16.

32. The nucleic acid according to claim 31, wherein the nucleic acid further comprises a nucleic acid sequence encoding a CH1 region and optionally a hinge, CH2 and CH3 region.

33. A cell comprising a nucleic acid sequence encoding a heavy chain variable region as defined in claim 16.

34. The cell according to claim 33, wherein the cell further comprises a nucleic acid sequence encoding a CH1 region and optionally a hinge, CH2 and CH3 region.

35. The cell according to claim 33, wherein the cell further comprises at least one nucleic acid sequence encoding a light chain variable region.

36. The cell according to claim 35, wherein the light chain variable region comprises light chain CDR1 (LCDR1), light chain CDR2 (LCDR2), and light chain CDR3 (LCDR3), having the amino acid sequence of SEO ID NO: 219, SEO ID NO: 220, and SEO ID NO: 221, respectively, wherein each of the LCDRs may comprise at most three, two, or one amino acid substitutions, and optionally a CL region.

37. A cell producing a binding domain of claim 1.

38. A kit comprising a container comprising a binding domain as claimed in claim 1, and optionally instructions for use.

39. A pharmaceutical composition comprising an effective amount of a binding moiety according to claim 21, and a pharmaceutically acceptable carrier.
